(19) United States
(12) Patent Application Publication
(10) Pub. No.: US 2021/0324377 A1
(43) Pub. Date: Oct. 21, 2021

(54) DEPLETING UNWANTED RNA SPECIES

(71) Applicant: QIAGEN Sciences, LLC, Germantown, MD (US)

(72) Inventors: Jonathan Shaffer, Hagerstown, MD (US); Eric Lader, Boyds, MD (US); Niels Tolstrup, Kokkedal (DK); Joerg Krummheuer, Holte (DK); Daniel Y. Kim, Brunswick, MD (US); Sascha Strauss, Hilden (DE)

(21) Appl. No.: 17/276,619

(22) PCT Filed: Sep. 19, 2019
(86) PCT No.: PCT/US2019/051999

§ 371 Date: Mar. 16, 2021

Related U.S. Application Data
(60) Provisional application No. 62/736,006, filed on Sep. 25, 2018.

(51) Int. Cl. C12N 15/10; C12Q 1/6848

(52) U.S. Cl. CPC C12N 15/1068; C12Q 1/6848

(57) ABSTRACT

The present disclosure provides methods and kits for inhibiting cDNA synthesis of unwanted RNA species during reverse transcription. The methods and kits provided herein use blocking oligonucleotides such as those comprising locked nucleic acids (LNAs).

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 830109_416WO_SEQUENCE_LISTING.txt. The text file is 97.5 KB, was created on Sep. 15, 2019, and is being submitted electronically via EFS-Web.

### BACKGROUND

### Technical Field

The present disclosure relates to methods and kits for depleting unwanted RNA species from RNA samples, especially for constructing transcriptome sequencing libraries.

### Description of the Related Art

Libraries constructed for transcriptome sequencing are heavily composed of unwanted species (e.g., cytoplasmic ribosomal RNA, mitochondrial ribosomal RNA, and globin mRNA) that take up a majority of the sequencing budget and render RNA sequencing extremely inefficient. rRNA alone constitutes greater than 80% of the RNA found a sample. As a result, various methods have been developed to enrich for mRNA or deplete unwanted RNA from next generation sequencing (NGS) libraries. For example, poly(A) RNA is isolated from RNA samples. While effective, this procedure is laborious and does not allow for the characterization of long non-coding RNAs or other RNAs which lack poly-A tails. In addition, it is unsuitable for heavily damaged samples, such as FFPE samples. Other methods use antisense DNA or RNA probes to hybridize unwanted RNAs in RNA samples prior to NGS library construction. After hybridization, in one approach, the samples are digested with a double stranded RNA specific enzyme (RNAase H), thus removing RNA probes and unwanted RNAs. However, this method is not very efficient and is fraught with technical uncertainties. In an alternative approach, the probes are biotinylated probes, allowing unwanted RNAs to be selectively removed out of the samples by capturing the probe/target RNA molecules to streptavidin coated beads or surfaces. However, this method is time consuming, costly, and only somewhat effective. In addition, the bead binding and washing is arduous and usually results in significant sample loss due to non-specific binding and capture.

### SUMMARY OF THE PRESENT DISCLOSURE

The present disclosure provides methods, blocking oligonucleotides, compositions, and kits for depleting unwanted RNA species from RNA samples.

In one aspect, the present disclosure provides a method for inhibiting cDNA synthesis of one or more unwanted RNA species in an RNA sample during reverse transcription, comprising:

(a) providing an RNA sample that comprises one or more desired RNA species and one or more unwanted RNA species,

(b) annealing one or more blocking oligonucleotides to one or more regions of the one or more unwanted RNA species in the RNA sample to generate a template mixture,

wherein the one or more blocking oligonucleotides are complementary, and stably bind, to the one or more regions of the one or more unwanted RNA species, and comprise 3′ modifications that prevent the one or more blocking oligonucleotides from being extended, and

(c) incubating the template mixture with a reaction mixture that comprises:

(i) at least one reverse transcriptase,

(ii) one or more reverse transcription primers, and

(iii) a reaction buffer,

under conditions sufficient to synthesize cDNA molecules using the one or more desired RNA species as template(s), wherein cDNA synthesis using the one or more unwanted RNA species is inhibited.

In another aspect, the present disclosure provides a set of blocking oligonucleotides that are complementary (preferably fully complementary) to a plurality of regions of an unwanted RNA species, wherein each blocking oligonucleotide comprises one or more modified nucleotides that increase its binding to a region of the unwanted RNA species.

In a related aspect, the present disclosure provides a plurality of sets of blocking oligonucleotides.

In another aspect, the present disclosure provides a kit of inhibiting cDNA synthesis of one or more unwanted RNA species in an RNA sample, comprising:

(1) (a) one or more blocking oligonucleotides that are complementary to one or more regions of one or more unwanted RNA species in the RNA sample, and each comprise one or more modified nucleotides that increase the binding between the one or more blocking oligonucleotides and the regions of the one or more unwanted RNA species, or
(b) the set of plurality of sets of blocking oligonucleotides provided herein, and

(2) a reverse transcriptase.

In another aspect, the present disclosure provides a method for designing blocking oligonucleotides for inhibiting cDNA synthesis of one or more unwanted RNA species in an RNA sample during reverse transcription, comprising:

(a) generating multiple blocking oligonucleotides complementary to regions of the one or more unwanted RNA species,

(b) filtering unacceptable blocking oligonucleotides,

(c) generating one or more groups of blocking oligonucleotides that are complementary to multiple different regions of the one or more unwanted RNA species, and

(d) optionally shuffling blocking oligonucleotides among the groups to generate new groups of blocking oligonucleotides and selecting one or more of the new groups of blocking oligonucleotides.

In another aspect, the present disclosure provides use of the kit of any of claims 28 to 43 or component (1) thereof in inhibiting cDNA synthesis of one or more unwanted RNA species in an RNA sample.

### BRIEF DESCRIPTION OF THE DRAWINGS

FIG. 1 is a scatter plot comparing relative gene expression for non-rRNA genes between using the Ribo-Zero rRNA Removal kit (Illumina) and blocking oligonucleotides (Blockers B1 to B193) in depleting unwanted RNA species according to Example 2.

FIG. 2 is a scatter plot comparing relative gene expression for non-rRNA genes between using blocking oligonucleotides (Blockers B1 to B193) and poly-A selection in depleting unwanted RNA species according to Example 2.

FIG. 3 is a scatter plot comparing relative gene expression for non-rRNA genes between using the Ribo-Zero rRNA Removal kit (Illumina) and poly-A in depleting unwanted RNA species according to Example 2.

FIG. 4 is a scatter plot comparing relative gene expression for non-rRNA genes between using the Ribo-Zero rRNA Removal kit (Illumina) in depleting unwanted RNA species and no depletion according to Example 2.

FIG. 5 is a scatter plot comparing relative gene expression for non-rRNA genes between using blocking oligonucleotides (Blockers B1 to B193) in depleting unwanted RNA species and no depletion according to Example 2.

FIG. 6 describes an exemplary algorithm for designing blockers as described in Example 4.

FIG. 7 is a graph showing the relationship between the number of blockers and the fraction of target 5S rRNA covered by the blockers as described in Example 4.

FIG. 8 is a graph showing the relationship between the number of blockers and the fraction of target 16S rRNA covered by the blockers as described in Example 4.

FIG. 9 is a graph showing the relationship between the number of blockers and the fraction of target 23S rRNA covered by the blockers as described in Example 4.

### DETAILED DESCRIPTION

The present disclosure provides methods, blocking oligonucleotides, compositions, and kits for depleting unwanted RNA species from RNA samples. The resulting depleted RNA samples are useful for various downstream applications, especially for constructing transcriptome sequencing libraries.

The methods provided herein use blocking oligonucleotides complementary to regions of unwanted RNA species (e.g., locked nucleic acid (LNA)-enhanced antisense oligonucleotides) to inhibit cDNA synthesis of the unwanted RNA species during reverse transcription.

Also disclosed are methods for designing tiled blocking oligonucleotides (e.g., LNA-enhanced antisense oligonucleotides), along an undesired RNA (e.g., cytoplasmic and mitochondrial rRNA, globin mRNA) at designated positions. The LNA bases are positioned in the oligonucleotides to facilitate the persistent binding of the antisense oligonucleotides to the unwanted RNA at commonly used reverse transcription temperatures.

The methods for depleting unwanted RNA species provided herein have one or more of the following advantages compared to existing methods: (1) because unwanted RNA depletion according to the present methods occurs during, rather than prior to, NGS library construction, they are faster and take fewer steps; (2) the present methods can be used not only with anchored oligo(dT) primed libraries, but also with random hexamer primed libraries; (3) the present methods can be used to deplete any unwanted RNAs (as opposed to enriching only poly(A)-containing RNAs using oligo(dT)); (4) the present methods do not significantly alter the remaining RNA profile of the samples (as opposed to poly(A) mRNA enrichment using oligo(dT)); (5) the present methods are more effective than or at least as effective as existing methods in depleting unwanted RNAs; and (6) the present methods cause less sample loss (e.g., compared to rRNA removal using biotin-labeled antisense oligonucleotides and streptavidin coated magnetic beads).

In the following description, any ranges provided herein include all the values in the ranges. It should also be noted that the term “or” is generally employed in its sense including “and/or” (i.e., to mean either one, both, or any combination thereof of the alternatives) unless the content dictates otherwise. Also, as used in this specification and the appended claims, the singular forms “a,” “an,” and “the” include plural referents unless the content dictates otherwise. The terms “include,” “have,” “comprise” and their variants are used synonymously and to be construed as non-limiting. The term “about” refers to ±10% of a reference a value. For example, “about 50° C.” refers to “50° C.±5° C.” (i.e., 50° C.±10% of 50° C.).

### A. Methods for Depleting Unwanted RNA Species

In one aspect, the present disclosure provides a method for inhibiting cDNA synthesis of one or more unwanted RNA species in an RNA sample during reverse transcription, comprising:

(a) providing an RNA sample that comprises one or more desired RNA species and one or more unwanted RNA species,

(b) annealing one or more blocking oligonucleotides to one or more regions of the one or more unwanted RNA species in the RNA sample to generate a template mixture,

wherein the one or more blocking oligonucleotides are complementary, and stably bind, to the one or more regions of the one or more unwanted RNA species, and comprise 3′ modifications that prevent the one or more blocking oligonucleotides from being extended, and

(c) incubating the template mixture with a reaction mixture that comprises:
(i) at least one reverse transcriptase,
(ii) one or more reverse transcription primers, and
(iii) a reaction buffer,

under conditions sufficient to synthesize cDNA molecules using the one or more desired RNA species as template(s), wherein cDNA synthesis using the one or more unwanted RNA species is inhibited.

1. Inhibiting cDNA Synthesis

cDNA synthesis of an RNA species is inhibited if the amount of single stranded or double stranded cDNA generated using the RNA species as a template during reverse transcription is reduced at a statistically significant degree under a modified condition (e.g., in the presence of one or more blocking oligonucleotides complementary to one or more regions of the RNA species) compared to the amount of single stranded or double stranded cDNA generated during reverse transcription under a reference condition (e.g., in the absence of the one or more blocking oligonucleotides).

The reduction in the amount of synthesized cDNA may be measured using qPCR or transcriptome sequencing as disclosed in the Examples provided herein, and may also include other techniques known to those skilled in the art (e.g., DNA microarrays).

The inhibition of cDNA synthesis of an RNA species may be referred to as depletion of the RNA species or as depleting the RNA species. Even though the RNA species is not physically removed from an initial RNA sample, the involvement of the RNA species in the downstream manipulation or analysis of the initial RNA sample is reduced or eliminated due to the inhibition of cDNA synthesis of the RNA species.

2. Unwanted RNA Species

The term “unwanted RNA species,” “unwanted RNAs,” or “unwanted RNA molecules” refers to RNA species or molecules undesired in an initial RNA composition for a given downstream manipulation or analysis of the RNA composition. Such RNA species or molecules are not the targets of, but may interfere with, downstream manipulation or analysis.

The unwanted RNA may be any undesired RNA present in the initial RNA composition. The unwanted RNA may comprise any sequence as long as it is distinguishable by its sequence from the remaining RNA population of interest to allow a sequence-specific design of blocking oligonucleotides.

According to one embodiment, the unwanted RNA is selected from one or more of the group consisting of rRNA, tRNA, snRNA, snoRNA and abundant protein mRNA.

When processing eukaryotic samples, the unwanted RNA may be an eukaryotic rRNA, preferably selected from 28S rRNA, 18S rRNA, 5.8S rRNA, 5S rRNA, mitochondrial 12S rRNA and mitochondrial 16S rRNA. Preferably, at least two, at least three, more preferred at least four of the aforementioned rRNA types are depleted, wherein preferably 18S rRNA and 28S rRNA are among the rRNAs to be depleted. According to one embodiment, all of the aforementioned rRNA types are depleted. Furthermore, it is preferred to also deplete other non-coding rRNA species, such as 12S and 16S eukaryotic mitochondrial rRNA molecules in addition to the 28S rRNA and 18S rRNA. In the cases where total RNA from plant samples are processed, plastid rRNA, such as chloroplast rRNA, may be depleted.

In certain embodiments, unwanted RNA(s) is one or more selected from the group consisting of 23S, 16S and 5S prokaryotic rRNA. This is particularly feasible when processing a prokaryotic sample. Preferably, all these rRNA types are depleted using one or more groups of blocking oligonucleotides specific for the respective rRNA type.

Furthermore, the methods of the present disclosure may also be used to specifically deplete abundant protein-coding mRNA species. Depending on the processed sample, mRNA comprised in the sample may correspond predominantly to a certain abundant mRNA type. For example, when intending to analyze, for example, sequence the transcriptome of a blood sample, most of the mRNA comprised in the sample will correspond to globin mRNA. However, for many applications, the sequence of the comprised globin mRNA is not of interest and thus, globin mRNA, even though being a protein-coding mRNA, also represents an unwanted RNA for this application. Additional unwanted, abundant protein-coding mRNAs may include ACTB, B2M, GAPDH, GUSB, HPRT1, HSP90AB1, LDHA, NONO, PGK1, PPIH, RPLP0, TFRC or various mitochondrial genes.

In certain embodiments, as described below, the RNA sample may be derived from (e.g., isolated from) a starting material that contains nucleic acids from multiple organisms, such as an environmental sample that contains plant, animal, and/or bacterial species or a clinical sample that contains human cells or tissues and one or more bacterial species. In such embodiments, unwanted RNA species may encompass or consist of a specific type of RNA species (e.g., 5S rRNA) from multiple organisms (e.g., multiple different bacteria) present in the starting material so that the method is capable of inhibiting cDNA synthesis of the specific type of RNA species from the multiple organisms (e.g., inhibiting cDNA synthesis of 5S rRNA from multiple bacteria in a starting material). In some other embodiments, unwanted RNA species may encompass or consist of multiple types of RNA species (e.g., 5S, 16S and 23S rRNAs) from multiple organisms (e.g., multiple different bacteria) present in the starting material so that the method is capable of inhibiting cDNA synthesis of multiple types of RNA species from the multiple organisms (e.g., inhibiting cDNA synthesis of 5S rRNA from multiple bacteria in a starting material).

In certain embodiments, the number of different unwanted RNA species to which blocking oligonucleotides are complementary is at least 2, at least 3, at least 4, or at least 5, at least 10, at least 20, at least 30, at least 40, at least 50, at least 75, at least 100, at least 200, at least 300, at least 400, or at least 500, and/or at most 1,000,000, at most 500,000, at most 100,000, at most 50,000, at most 10,000, at most 9000, at most 8000, at most 7000, at most 6000, at most 5000, at most 4000, at most 3000, or at most 2000, such as from 2 to 1,000,000, from 100 to 500,000, from 500 to 100,000, and from 1000 to 10,000.

3. RNA Sample

As described above, step (a) of a method for inhibiting cDNA synthesis of one or more unwanted RNA species in an RNA sample during reverse transcription disclosed herein is to provide an RNA sample that comprises one or more desired RNA species and one or more unwanted RNA species.

The term “RNA sample” refers to an RNA-containing sample. Preferably, an RNA sample is a sample containing RNAs isolated from a starting material. An RNA sample may further contain DNAs isolated from the starting material. In some embodiments, an RNA sample contains RNA molecules that have been isolated from a starting material and further fragmented. In other cases, an RNA sample is derived from a directly lysed sample without specific nucleic acid isolation.

The term “nucleic acid” or “nucleic acids” as used herein refers to a polymer comprising ribonucleosides or deoxyribonucleosides that are covalently bonded typically by phosphodiester linkages between subunits. Nucleic acids include DNA and RNA. DNA includes but is not limited to genomic DNA, linear DNA, circular DNA, plasmid DNA, cDNA and free circulating DNA (e.g., tumor derived or fetal DNA). RNA includes but is not limited to hnRNA, mRNA, noncoding RNA (ncRNA), and free circulating RNA (e.g., tumor derived RNA). Noncoding RNA includes but is not limited to rRNA, tRNA, lncRNA (long non coding RNA), lincRNA (long intergenic non coding RNA), miRNA (micro RNA), and siRNA (small interfering RNA),

The starting material from which the RNA sample is generated can be any material that comprises RNA molecules. The starting material can be a biological sample or material, such as a cell sample, an environmental sample, a sample obtained from a body, in particular a body fluid sample, and a human, animal or plant tissue sample. Specific examples include but are not limited to whole blood, blood products, plasma, serum, red blood cells, white blood cells, buffy coat, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, pulmonary lavage, bone marrow aspirates, lung aspirates, biopsy samples, swab samples, animal (including human) or plant tissues, including but not limited to samples from liver, spleen, kidney, lung, intestine, brain, heart, muscle, pancreas, cell cultures, as well as lysates, extracts, or materials and fractions obtained from the samples described above or any cells and microorganisms and viruses that may be present on or in a sample and the like.

Materials obtained from clinical or forensic settings that contain RNA are also within the intended meaning of a starting material. Preferably, the starting material is a biological sample derived from a eukaryote or prokaryote, preferably from human, animal, plant, bacteria or fungi. Preferably, the starting material is selected from the group consisting of cells, tissue, tumor cells, bacteria, virus and body fluids such as blood, blood products (e.g., buffy coat, plasma and serum), urine, liquor, sputum, stool, CSF and sperm, epithelial swabs, biopsies, bone marrow samples and tissue samples, preferably organ tissue samples such as lung, kidney or liver.

The starting material also includes processed samples such as preserved, fixed and/or stabilised samples. Non-limiting examples of such samples include cell containing samples that have been preserved, such as formalin fixed and paraffin-embedded (FFPE samples) or other samples that were treated with cross-linking or non-crosslinking fixatives (e.g., glutaraldehyde) or the PAXgene Tissue system. For example, tumor biopsy samples are routinely stored after surgical procedures by FFPE, which may compromise the RNA integrity and may in particular degrade the comprised RNA. Thus, an RNA sample may consist of or comprise modified or degraded RNA. The modification or degradation can be due to, for example, treatment with a preservative(s).

Nucleic acids can be isolated from a starting material according to methods known in the art to provide an RNA sample. The RNA sample may contain both DNA and RNA. In certain embodiments, the RNA sample contains predominantly RNA as DNA in the starting material has been removed or degraded. RNA in an RNA sample may be total RNA isolated from a starting material. Alternatively, RNA in an RNA sample may be a fraction of total RNA (e.g., the fraction containing mostly mRNA) isolated from a starting material where certain RNA species (e.g., RNA without a poly(A) tail) have been depleted or removed.

As disclosed above, an RNA sample may contain RNA molecules that have been isolated from a starting material and further fragmented. Fragmenting nucleic acids, such as isolated RNAs, may be performed physically, enzymatically or chemically. Physical fragmentation includes acoustic shearing, sonication, and hydrodynamic shearing. Enzymatic fragmentation may use an endonuclease (e.g., RNase III) that cleaves RNA into small fragments with 5′ phosphate and 3′ hydroxyl groups. Chemical fragmentation includes heat and divalent metal cation (e.g., magnesium or zinc).

Also as disclosed above, in certain embodiments, an RNA sample is from a crude lysate where specific nucleic acid isolation has not been performed.

4. Desired RNA Species

In addition to unwanted RNAs, an RNA sample also contains one or more desired RNA species. Desired RNA species can be any RNA species or molecules characteristic(s) of which (e.g., expression level or sequence) are of interest. In certain embodiments, the desired RNA species comprise mRNA, preferably those of which expression level changes (compared with a reference expression level) or sequence changes (compared with wild type sequences) are associated with a disease or disorder or with responsiveness to a treatment of a disease or disorder.

5. Blocking Oligonucleotides

The term “blocking oligonucleotide” as used herein refers to an oligonucleotide that is complementary and capable of stably binding to a region of an unwanted RNA species. The blocking oligonucleotide may be described as “targeting” the region of the unwanted RNA species. The blocking oligonucleotide is incapable of being extended due to a modification at its 3′ terminus (i.e., “3′ modification”). Consequently, the blocking oligonucleotide is able to inhibit cDNA synthesis using the region of the unwanted RNA species as a template during reverse transcription.

An oligonucleotide is capable of stably binding to a region of a RNA species if the oligonucleotide anneals to the region of the RNA species and stays bound to the region of the RNA species during reverse transcription of a RNA sample comprising the RNA species.

Preferably, a blocking oligonucleotide contains one or more modified nucleotides that increase the binding between the oligonucleotide and the region of the unwanted RNA species compared to an oligonucleotide with the same sequence but without any modified nucleotides. In certain other embodiments, a blocking oligonucleotide does not contain any of the above-described modified nucleotides, but is sufficiently long to be able to stably bind to a region of the unwanted RNA species during reverse transcription.

In the embodiments where a blocking oligonucleotide contains one or more modified nucleotides that increase the binding between the oligonucleotide and the region of an unwanted RNA species, the region of the unwanted RNA species to which the blocking oligonucleotide is complementary may be at least 10 nucleotides in length, such as at least 11, 12, 13, 14, 15, 16, 17, or 18 nucleotides in length. Such a region may be at most 100 nucleotides in length, such as at most 90, 80, 70, 60, 50, 45, 40, 35, 30, 25, 24, 23, 22, 21, or 20 nucleotides in length. In certain embodiments, the region may be 10 to 100 nucleotides in length, such as 15 to 80, 20 to 60, 25 to 40, 10 to 30, 16 to 24, or 18 to 22 nucleotides in length.

In the embodiments where a blocking oligonucleotide does not contain any modified nucleotides that increase the binding between the oligonucleotide and the region of an unwanted RNA species, the region of the unwanted RNA species to which the blocking oligonucleotide is complementary may be at least 20, 21, 22, 23, 24, 25, 26, 27, 28, 29, 30, 31, 32, 33, 34, 35, 36, 37, 38, 39, or 40 nucleotides in length. Such a region may be at most 100 nucleotides in length, such as at most 90, 80, 70, 60, or 50 nucleotides in length. In certain embodiments, the region may be 20 to 100 nucleotides in length, such as 25 to 90, 25 to 80, 25 to 70, 25 to 60, 25 to 50, 25 to 40, 25 to 30, 30 to 90, 30 to 80, 30 to 70, 30 to 60, 30 to 50, 30 to 40, 35 to 90, 35 to 80, 35 to 70, 35 to 60, 35 to 50, 35 to 40, 40 to 90, 40 to 80, 40 to 70, 40 to 60, or 40 to 50 nucleotides in length.

As disclosed above, a blocking oligonucleotide is complementary to a region of an unwanted RNA species. An oligonucleotide is complementary to a region of an unwanted RNA species if at least 80%, such as at least 85%, at least 90% or preferably at least 95% of nucleotides in the oligonucleotide are complementary to the region of the unwanted RNA species. In certain embodiments, a blocking oligonucleotide comprises one or more (e.g., at most 6, at most 5, at most 4, at most 3, at most 2, or only 1) nucleotide mismatches with the region of the unwanted RNA species. Preferably, the mismatch is at or near (e.g., within the first 10 nucleotides, such as within the first 5 nucleotides, from) the 5′ terminus of the oligonucleotide. For example, a blocking oligonucleotide having the sequence of 5′-GACAAACCCTTGTGTCGAG-3′ (SEQ ID NO: 15) is complementary to the region of 3′-GTCGACACAAGGGTTTGTC-5′ (SEQ ID NO: 508) of an unwanted RNA species even though there is a mismatch between the 5′ terminal “G” of the oligonucleotide and the 3′ terminal “G” of the region of the unwanted RNA species. In certain other embodiments, a blocking oligonucleotide may comprise a one or more nucleotide-insertion (e.g., an insertion having at most 6, at most 5, at most 4, at most 3, at most 2, or only 1 nucleotide) when compared with the fully complementary sequence of the region of the unwanted RNA species. For example, a blocking oligonucleotide may comprise two segments that are fully complementary to two contiguous sections of a region of an unwanted RNA species respectively, but are separated by one or more nucleotides.

Preferably, a blocking oligonucleotide is fully complementary to a region of an unwanted RNA species. An oligonucleotide is fully complementary to a region of an unwanted RNA species if each nucleotide of the oligonucleotide is complementary to a nucleotide at the corresponding position in the region of the unwanted RNA species. For example, an oligonucleotide having the sequence of 5′-GACAAACCCTTGTGTCGAG-3′ (SEQ ID NO: 15) is fully complementary to the region of 3′-CTCGACACAAGGGTTTGTC-5′ (SEQ ID NO: 509) of an unwanted RNA species.

Also as disclosed above, a blocking oligonucleotide has a 3′ modification that prevents the oligonucleotide from being extended during reverse transcription. The 3′ modification replaces the 3′-OH of an oligonucleotide with another group (e.g., a phosphate group), which rendering the resulting oligonucleotide incapable of being extended by a reverse transcriptase during reverse transcription. 3′ modifications that prevent oligonucleotides that contain such modifications from being extended include but are not limited to 3′ ddC (dideoxycytidine), 3′ inverted dT, 3′ C3 spacer, 3′ Amino Modifier (3AmMo), and 3′ phosphorylation. Some of 3′ modifications are commercially available, such as from Integrated DNA Technologies.

a. Blocking Oligonucleotides Having Modified Nucleotides for Increasing Binding

As disclosed above, preferably, a blocking oligonucleotide comprises one or more modified nucleotides that increase the binding between the blocking oligonucleotide and a region of an unwanted RNA species to which the blocking oligonucleotide is complementary compared to an oligonucleotide with the same sequence but without any modified nucleotide.

Modified nucleotides are nucleotides other than naturally occurring nucleotides that each comprise a phosphate group, a 5-carbon sugar (i.e., deoxyribose or ribose), and a nitrogenous base selected from adenine, cytosine, guanine, thymine and uridine.

A modified nucleotide that increases the binding between an oligonucleotide and a region of an unwanted RNA species compared to an oligonucleotide with the same sequence but without any modified nucleotides if it increases the melting temperature of the duplex formed between the oligonucleotide comprising the modified nucleotide and the region of the unwanted RNA species compared to the melting temperature of the duplex formed between the oligonucleotide with the same sequence but without any modified nucleotides and the region of the unwanted RNA species measured under the same conditions (e.g., in 20 mM KCl).

The melting temperature (Tm) of an oligonucleotide as used in the present disclosure is the temperature at which 50% of the oligonucleotide is duplexed with its perfect complement and 50% is free in 115 mM KCl. Tm is determined by measuring the absorbance change of the oligonucleotide with its complement as a function of temperature (i.e., generating a melting curve). The Tm is the reading halfway between the double-stranded DNA and single stranded DNA plateaus in the melting curve.

Exemplary nucleotides capable of increasing Tm of oligonucleotides that comprise such nucleotides include but are not limited to nucleotides comprising 2′-O-methylribose, 5-hydroxybutynyl-2′-deoxyridine (Integrated DNA Technologies), 2-Amino-2′deoxyadenosine (IBA Lifesciences), 5-Methyl-2′deoxycytidine (IBA Lifesciences), or locked nucleic acids (LNA).

Preferably, blocking oligonucleotides comprise one or more LNAs. LNA is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide and hybridize with DNA or RNA according to Watson-Crick base-pairing rules. The locked ribose conformation enhances base stacking and backbone pre-organization. This significantly increases the hybridization properties (melting temperature) of oligonucleotides (see e.g., Kaur et al., Biochemistry 45(23): 7347-55, 2006; Owczarzy et al., Biochemistry 50(43): 9352-67, 2011). An increase in the duplex melting temperature can be 2-8° C. per LNA nucleotide when incorporated into an oligonucleotide. DNA or RNA oligonucleotides that comprise one or more LNA nucleotides are referred to as “LNA oligonucleotides.” Such oligonucleotides can be synthesized by conventional phosphoamidite chemistry and are commercially available (e.g., from Exiqon).

Additional blocking oligonucleotides may be peptide nucleic acid oligomers that are synthetic polymers similar to DNA or RNA but with backbone composed of repeating N-(2-aminoethyl)-glycine units linked by peptide bonds. In peptide nucleic acid oligomers, various purine and pyrimidine bases are linked to the backbone by a methylene bridge (—CH₂—) and a carbonyl group (—(C═O)—).

The number of modified nucleotides (e.g., LNAs) in a blocking oligonucleotide ranges from 3 to 30, preferably 4 to 16, more preferably 3 to 15.

The lengths of blocking oligonucleotides may be at least 10 nucleotides in length, such as at least 11, 12, 13, 14, 15, 16, 17, or 18 nucleotides in length. They may be at most 100 nucleotides, such as at most 100 nucleotides in length, such as at most 90, 80, 70, 60, 50, 45, 40, 35, 30, 25, 24, 23, 22, 21, or 20 nucleotides in length. In certain embodiments, the lengths may be 10 to 100 nucleotides, such as 15 to 80, 20 to 60, 25 to 40, 10 to 30, 16 to 24, or 18 to 22 nucleotides.

The melting temperature of duplexes formed between blocking oligonucleotides and regions of unwanted RNA species to which the blocking oligonucleotides are complementary range from 80 to 96° C., 82 to 94° C., or preferably 86 to 92° C. as measured in 115 mM KCl.

b. Blocking Oligonucleotides without Modified Nucleotides for Increasing Binding

As disclosed above, in certain embodiments, a blocking oligonucleotide does not comprise any modified nucleotides that increase the binding between the blocking oligonucleotide and a region of an unwanted RNA species to which the blocking oligonucleotide is complementary, but is sufficiently long to be able to stably bind to a region of the unwanted RNA species during reverse transcription.

The lengths of blocking oligonucleotides without the above-described modified nucleotides may be at least 20 nucleotides in length, such as at least 25, 26, 27, 28, 29, 30, 31, 32, 33, 34, or 35 nucleotides in length. They may be at most 100 nucleotides, such as at most 90, 80, 70, 60, 50, 45, or 40 nucleotides in length. In certain embodiments, the lengths may be 25 to 100 nucleotides, such as 30 to 80, 30 to 70, 30 to 60, 30 to 50, 30 to 45, 30 to 40, 35 to 80, 35 to 70, 35 to 60, 35 to 50, 35 to 45, 40 to 80, 40 to 70, 40 to 60, 40 to 50, or 40 to 45 nucleotides.

The melting temperature of duplexes formed between blocking oligonucleotides and regions of unwanted RNA species to which the blocking oligonucleotides are complementary range from 80 to 96° C., 82 to 94° C., or preferably 86 to 92° C. as measured in 115 mM KCl.

c. Multiple Blocking Oligonucleotides

The number of blocking oligonucleotides used in the method disclosed herein may be at least 2, at least 3, at least 4, at least 5, at least 10, at least 50, at least 100, at least 150, at least 200, at least 300, at least 400, at least 500, at least 600, at least 700, at least 800, at least 900, at least 1000, at least 1500, or at least 2000, at least 3000, at least 4000, at least 5000, at least 6000, at least 7000, at least 8000, at least 9000, or at least 10,000, and/or at most 100,000, at most 90,000, at most 80,000, at most 70,000, at most 60,000, or at most 50,000, such as from 2 to 100,000, from 100 to 80,000, or from 800 to 50,000.

In certain embodiments, 2 or more blocking oligonucleotides are complementary to multiple different regions (e.g., at least 2, at least 3, at least 4, at least 5, at least 6, at least 7, at least 8, at least 9, or at least 10) of a single unwanted RNA species. In certain other embodiments, 2 or more blocking oligonucleotides are complementary to multiple different regions (e.g., at least 2, at least 3, at least 4, at least 5, at least 6, at least 7, at least 8, at least 9, or at least 10 different regions) of multiple unwanted RNA species (e.g., at least 2, at least 3, at least 4, at least 5, at least 6, at least 7, at least 8, at least 9, or at least 10 unwanted RNA species).

In certain embodiments where multiple blocking oligonucleotides are complementary to multiple different regions of one or more unwanted RNA species, the distances between two neighboring regions of the one or more unwanted RNA species to which the blocking oligonucleotides are complementary may range from 0 to 100 nucleotides, such as 0 to 75 nucleotides, 0 to 50 nucleotides, 20 to 100 nucleotides, 20 to 75 nucleotides, 20 to 50 nucleotides, 30 to 100 nucleotides, 30 to 75 nucleotides, 30 to 50 nucleotides, or 30 to 45 nucleotides.

In certain embodiments, the blocking oligonucleotides comprise or consist of a set of blocking oligonucleotides for inhibiting cDNA synthesis of a single unwanted RNA species (e.g., E. coli 5S rRNA). The blocking oligonucleotides are complementary to multiple different (preferably evenly spaced as described in detail in other sections below) regions of the unwanted RNA species.

In certain other embodiments, the blocking oligonucleotides comprise or consist of a plurality of sets of blocking oligonucleotides for inhibiting cDNA synthesis of multiple unwanted RNA species. Each set of blocking oligonucleotides are complementary to multiple different (preferably evenly spaced) regions of an unwanted RNA species as described above, and different sets of blocking oligonucleotides are complementary to evenly spaced regions of different unwanted RNA species.

Blocking oligonucleotides may also be referred herein as “blockers,” “blocking antisense oligonucleotides,” or the like.

Exemplary blocking oligonucleotides (Blockers B1 to B193) that can be used in depleting human 18S rRNA in the method according to the present disclosure are described in the Examples. Exemplary blocking oligonucleotides (Blockers 5S1 to 5S100, Blockers 16S1 to 16S100, Blockers 23S1 to 23S100) that can be used in depleting bacterial 5S, 16S, and 23S rRNAs, respectively, are described in Example 4.

Additional descriptions of blocking oligonucleotides are provided in Sections B, C and D of the present disclosure below.

6. Annealing Blocking Oligonucleotides to Unwanted RNAs

As disclosed above, step (b) of a method for inhibiting cDNA synthesis of one or more unwanted RNA species in an RNA sample during reverse transcription disclosed herein is to anneal one or more blocking oligonucleotides to one or more regions of one or more unwanted RNA species in the RNA sample to generate a template mixture.

This step may be performed by mixing an RNA sample with one or more blocking oligonucleotides under conditions appropriate for the blocking oligonucleotide(s) to anneal to the one or more regions of the one or more unwanted RNA species in the RNA sample. The resulting mixture is referred to herein as “annealing mixture.”

Typically, the annealing mixture is first heated to a high temperature (e.g., about 65° C., about 70° C., 75° C., 80° C., 85° C., 90° C., or 95° C., or at least 65° C., at least 70° C., preferably at least 75° C.) for a sufficient period of time (e.g., at least about 30 seconds, such as at least 1 minute or at least 2 minutes) so that the RNA molecules in the RNA sample is denatured, and then cooled down to a lower temperature (e.g., at or lower than 40° C., such as at or lower than 25° C., at or lower than room temperature (22° C. to 25° C.), or at 4° C.).

The cooling process may be performed in various ways, such as gradually reduced the temperature at defined levels for defined time periods or cooling down naturally to room temperature. Exemplary cooling processes include but are not limited to the following:

### Process 1

| Temperature | Time |
|---|---|
| 75° C. | 2 min |
| 70° C. | 2 min |
| 65° C. | 2 min |
| 60° C. | 2 min |
| 55° C. | 2 min |
| 37° C. | 5 min |
| 25° C. | 5 min |
| 4° C. | hold |

### Process 2

| Temperature | Time |
|---|---|
| 90° C. | 30 sec |
| 85° C. | 2 min |
| 80° C. | 2 min |
| 75° C. | 2 min |
| 70° C. | 2 min |
| 65° C. | 2 min |
| 60° C. | 2 min |
| 55° C. | 2 min |
| 37° C. | 5 min |

### Process 3

| Temperature | Time |
|---|---|
| 90° C. | 2 min |

Turn off thermocycler, let it cool down to room temperature

### Process 4

| Temperature | Time |
|---|---|
| 89° C. | 8 min |
| 75° C. | 2 min |
| 70° C. | 2 min |
| 65° C. | 2 min |
| 60° C. | 2 min |
| 55° C. | 2 min |
| 37° C. | 2 min |
| 25° C. | 2 min |

The amount of one or more blocking oligonucleotides in the annealing mixture may be from about 0.1 pmol to about 50 pmol per blocking oligonucleotide, such as from about 0.5 pmol to about 20 pmol, from about 0.5 pmol to about 10 pmol, from about 1 pmol to about 20 pmol, from about 1 pmol to about 10 pmol, from about 1.5 pmol to about 10 pmol, from about 1.5 pmol to about 8 pmol, or from 2 pmol to about 7 pmol per blocking oligonucleotide.

Preferably, about the same amount of each of different blocking oligonucleotides is present in the anneal mixture. In certain embodiments, the amounts of different blocking oligonucleotides are different. For example, the molar ratio of the blocking oligonucleotide having the highest amount to that having the lowest amount may be from about 10 to about 1.1, about 5 to about 1.1, or about 2 to about 1.1.

The amount of RNA from in the annealing mixture may range from about 1 pg to about 5000 ng, such as from about 5 pg to about 5000 ng, about 10 pg to about 5000 ng, about 100 pg to about 5000 ng, about 1 ng to about 5000 ng, about 5 ng to about 5000 ng, about 10 ng to about 5000 ng, about 100 ng to about 5000 ng, about 5 pg to about 3000 ng, about 10 pg to about 3000 ng, about 100 pg to about 3000 ng, about 1 ng to about 3000 ng, about 5 ng to about 3000 ng, about 10 ng to about 3000 ng, about 100 ng to about 3000 ng, about 5 pg to about 1000 ng, about 10 pg to about 1000 ng, about 100 pg to about 1000 ng, about 1 ng to about 1000 ng, about 5 ng to about 1000 ng, about 10 ng to about 1000 ng, about 100 ng to about 1000 ng, or from about 25 ng to about 500 ng. The amount of RNA may be at least about 1 pg, about 5 pg, about 10 pg, about 50 pg, about 100 pg, about 500 pg, about 1 ng, about 5 ng, about 10 ng, about 50 ng or about 100 ng and/or at most about 500 ng, about 1000 ng, about 3000 ng, or about 5000 ng.

The annealing mixture may contain, in addition to one or more blocking oligonucleotides and an RNA sample, one or more monovalent cations (e.g., Na⁺ and K⁺) to increase the annealing of the blocking oligonucleotides to unwanted RNA species. The monovalent concentration in the annealing mixture ranges from 5 mM to 50 mM, such as 10 mM to 30 mM or 15 mM to 25 mM.

Preferably, the annealing mixture contains NaCl or KCl at a concentration of 10 mM to 30 mM, such as 15 mM to 25 mM.

The annealing mixture may optionally comprise a buffer with a pH ranging from 5 to 9, such as a buffer containing 20-50 nM phosphate, pH 6.5 to 7.5.

Once the annealing process is performed, the annealing mixture may be referred to as “template mixture,” which will be used as templates for subsequent cDNA synthesis. In certain embodiments, the annealing mixture may be cleaned up before used as templates for cDNA synthesis. For example, the cleanup may be performed using a solid support that binds nucleic acid (e.g., RNA) by mixing the annealing mixture with the solid support, separating the solid support with nucleic acids bound thereto from the liquid phase, optionally washing the solid support, and eluting the nucleic acids from the solid support. This mixing, separating, optional washing and eluting process may be repeated once (i.e., two rounds of cleanup), twice (i.e., three rounds of cleanup), or more times. Exemplary solid support includes QIAseq beads as used in the Examples described below.

7. Reverse Transcription

As disclosed above, step (c) of a method for inhibiting cDNA synthesis of one or more unwanted RNA species in an RNA sample during reverse transcription disclosed herein is to incubate the template mixture generated as described above with a reaction mixture that comprises: (i) at least one reverse transcriptase, (ii) one or more reverse transcription primers, and (iii) a reverse transcription buffer under conditions sufficient to synthesize cDNA molecules using one or more desired RNA species as template(s). Because one or more blocking oligonucleotides anneal to one or more unwanted RNA species, the transcription of such unwanted RNA species are inhibited.

8. Reverse Transcriptase

The term “reverse transcriptase” refers to an RNA dependent DNA polymerase capable of synthesizing complementary DNA (cDNA) strand using an RNA template. Reverse transcriptases useful in step (c) may be one or more viral reverse transcriptase, including but not limited to AMV reverse transcriptase, RSV reverse transcriptase, MMLV reverse transcriptase, HIV reverse transcriptase, EIAV reverse transcriptase, RAV reverse transcriptase, TTH DNA polymerase, C. hydrogenoformans DNA polymerase, Superscript® I reverse transcriptase, Superscript® II reverse transcriptase, Thermoscript™ RT MMLV, ASLV and RNase H mutants thereof, or a mixture of some of the above enzymes. Preferably, the reverse transcriptase is EnzScript™ M-MLV Reverse Transcriptase RNA H-(Enzymatics), which contains three point mutations that eliminate measurable RNase H activity native to wild type M-MLV reverse transcriptase. Loss of RNase H activity enables greater yield of full-length cDNA transcripts (5 kb) and increased thermal stability over wild type M-MLV reverse transcriptase. Increased thermostability allows for higher incubation temperatures of the first-strand reaction (up to 50° C.), aiding in denaturation of template RNA secondary structure of GC-rich regions.

9. Reverse Transcription Primers

Reverse transcription primers useful in step (c) may be oligo(dT) primers, that is, single strand sequences of deoxythymine (dT). The length of oligo(dT) can vary from 8 bases to 30 bases and may be a mixture of oligo(dT) with different lengths such as oligo(dT)₁₂₋₁₈ or oligo(dT) with a single defined length such as oligo(dT)₁₈ or oligo(dT)₂₀.

Preferably, reverse transcription primers used in step (c) are random primers, such as random hexamers (N6), heptamers (N7), octamers (N8), nonamers (N9), etc.

In certain embodiments, reverse transcription primers may be a mixture of one or more oligo(dT) primers and one or more random primers.

In certain other embodiments, reverse transcription primers may comprise primers specific for one more desired RNA species.

The reverse transcription primers may be immobilized or anchored, such as anchored oligo(dT) primers. Alternatively, they may be in solution and not immobilized to a solid phase (e.g., beads).

10. Reaction Buffer and Other Components

The reaction mixture of step (c) (also referred to as “reverse transcription reaction mixture”) comprises a reaction buffer suitable for reverse transcription, such as a Tris buffer with pH about 8.3 or 8.4 at a concentration ranging from about 20 to about 50 mM.

The reaction mixture also comprises dNTPs at a concentration ranging from about 0.1 to about 1 mM (e.g., about 0.5 mM) each dNTP.

The reaction mixture typically also comprises MgCl₂ at a concentration ranging from about 1 to about 10 mM, such as about 3 to about 5 mM.

The reaction mixture optionally further comprises a reducing agent, such as DTT at a concentration ranging from about 5 to about 20 mM, such as about 10 mM.

11. Conditions for Reverse Transcription

The reaction mixture is subject to conditions sufficient to synthesize cDNA molecules using one or more desired RNA species in an RNA sample as templates. The conditions typically include incubating the reaction mixture at one or more appropriate temperatures (e.g., at about 35° C. to about 50° C. or about 37° C. to 45° C., such as at about 35° C., about 37° C., about 40° C., about 42° C., about 45° C., or about 50° C.) for a sufficient period of time (e.g., for about 30 minutes to about 1 hour). In certain embodiments, a low temperature incubation step (e.g., at 25° C. for about 2 to about 10 minutes) may be performed for primer extension to increase the primer Tm before a higher temperature incubation step for the first stand cDNA synthesis.

12. Synthesizing 2^{nd} cDNA Strands

In certain embodiments, after step (c) (i.e., the synthesis of the first strand cDNA), the method disclosed herein may comprise step (d) that synthesize the second strand cDNA to generate double stranded cDNA.

Procedures known in the art for synthesizing the second strand cDNA may be used in step (d). For example, E. Coli RNase H may be used to nick nicks and gaps of mRNA resulting from the endogenous RNase H of reverse transcriptase. Polymerase I then initiates second strand synthesis by nick translation. E. coli DNA ligase subsequently seals any breaks left in the second strand cDNA, generating double stranded cDNA products.

Step (d) may also be performed using QIAseq Stranded Total RNA Library kit (QIAGEN) or other commercially available kits (e.g., from Illumina, New England BioLabs, KAPA Biosystems, Thermo Fisher Scientific).

13. Constructing Sequencing Library and Sequencing

In certain embodiments, after double stranded DNA is generated in step (d), the method disclosed herein further comprises step (e) to amplify the double stranded cDNA generated in step (d) to construct a sequencing library. The sequencing library may be used to sequence the one or more desired RNA species in a further step, step (f).

The double stranded cDNA generated in step (d) may be used to prepare a sequencing library in step (e) using methods known in the art. For example, the double stranded DNA may be end-repaired, subject to A-addition, and ligated with adapters. The adapter-linked cDNA molecules may be further amplified via one or more rounds of amplification (e.g., universal PCR, bridge PCR, emulsion PCR, or rolling cycle amplification) to generate a sequencing library (i.e., a collection of DNA fragments that are ready to be sequenced, such as comprising a sequencing primer-binding site).

The sequencing library may be sequenced using methods known in the art in step (f) (see, Myllykangas et al., Bioinformatics for High Throughput Sequencing, Rodriguez-Ezpeleta et al. (eds.), Springer Science+Business Media, LLC, 2012, pages 11-25). Exemplary high throughput DNA sequencing systems include, but are not limited to, the GS FLX sequencing system originally developed by 454 Life Sciences and later acquired by Roche (Basel, Switzerland), Genome Analyzer developed by Solexa and later acquired by Illumina Inc. (San Diego, Calif.) (see, Bentley, Curr Opin Genet Dev 16:545-52, 2006; Bentley et al., Nature 456:53-59, 2008), the SOLiD sequence system by Life Technologies (Foster City, Calif.) (see, Smith et al., Nucleic Acid Res 38: e142, 2010; Valouev et al., Genome Res 18:1051-63, 2008), CGA developed by Complete Genomics and acquired by BGI (see, Drmanac et al., Science 327:78-81, 2010), PacBio RS sequencing technology developed by Pacific Biosciences (Menlo Park, Calif.) (see, Eid et al., Science 323: 133-8, 2009), and Ion Torrent developed by Life Technologies Corporation (see, U.S. Patent Application Publication Nos. 2009/0026082; 2010/0137143; and 2010/0282617).

Sequencing reads obtained from sequencing the sequencing library may be analyzed to determine the expression levels and/or sequences of RNA species of interest. Such information may be useful in diagnosing diseases or predicting responsiveness of the subjects from which the RNA samples are obtained to specific treatments.

14. Other Downstream Uses

The double stranded cDNA generated in step (d) may be used in microarray analysis to determine expression levels, including the presence or absence, of RNA species of interest. Additional uses include functional cloning to identify genes based on their encoded proteins' functions, discover novel genes, or study alternative slicing in different cells or tissues.

15. Depletion Efficiency

The first strand cDNA molecules may be used as templates in qPCR to check the efficiency of the blocking oligonucleotides in inhibiting cDNA synthesis from unwanted RNA species to which the blocking oligonucleotides are complementary. An exemplary method is disclosed in Example 1 below. Briefly, an increase in Ct of amplifying a cDNA reverse transcribed from an unwanted RNA species when one or more blocking oligonucleotides are used during reverse transcription compared with when no blocking oligonucleotides are used during reverse transcription indicates that the one or more blocking oligonucleotides are effective in inhibiting cDNA synthesis from the unwanted RNA species. The increase in Ct may be compared with that of another treatment (e.g., a commercially available treatment) to demonstrate equivalent to or improvement over the other treatment.

In certain embodiments, the Ct value of amplifying a cDNA reverse transcribed from an unwanted RNA species when one or more blocking oligonucleotides are used during reverse transcription is at least 2 times, at least 2.5 times, at least 3 times, or at least 4 times as much as the Ct value when no blocking oligonucleotides are used during reverse transcription.

The efficiency of the blocking oligonucleotides in inhibiting cDNA synthesis from unwanted RNA species may also be analyzed via whole transcriptome sequencing. An exemplary method is disclosed in Example 2 below. Briefly, the decrease in percentage of total reads that are derived from an unwanted RNA species (e.g., 18S rRNA) when one or more blocking oligonucleotides are used during reverse transcription compared with when no blocking oligonucleotides are used during reverse transcription indicates that the one or more blocking oligonucleotides are effective in inhibiting cDNA synthesis from the unwanted RNA species. The decrease in percentage may be compared with that of another treatment (e.g., a commercially available treatment) to demonstrate equivalent to or improvement over the other treatment.

The percentage of total reads that are derived from an unwanted RNA species (e.g., 18S rRNA) when one or more blocking oligonucleotides are used during reverse transcription according to the present disclosure may be at most 5%, at most 4%, at most 3%, at most 2%, at most 1%, at most 0.8%, at most 0.6%, at most 0.5%, at most 0.4%, at most 0.3%, at most 0.2%, at most 0.1% or at most 0.05%.

The ratio of the percentage of total reads that are derived from an unwanted RNA species (e.g., 18S rRNA) when one or more blocking oligonucleotides are used during reverse transcription to that when no blocking oligonucleotide are used may be at most 0.2, at most 0.15, at most 0.1, at most 0.08, at most 0.06, at most 0.05, at most 0.04, at most 0.03, or at most 0.02.

16. Off-Target Depletion

The first strand cDNA molecules may be used as templates in qPCR to check the degree of off-target depletion by blocking oligonucleotides. An exemplary method is disclosed in Example 1 below. Briefly, an increase in Ct of amplifying a cDNA reverse transcribed from a desired RNA species when one or more blocking oligonucleotides targeting one or more unwanted RNA species are used during reverse transcription compared with when no blocking oligonucleotides are used during reverse transcription indicates that the one or more blocking oligonucleotides cause inhibition of cDNA synthesis from the desired RNA species. Such inhibition is referred to “off-target depletion.” The increase in Ct may be compared with that of another treatment (e.g., a commercially available treatment) to evaluate off-target depletion of the two treatments.

In certain embodiments, the increase in Ct value of amplifying a cDNA reverse transcribed from a desired RNA species (e.g., GAPDH mRNA) between when one or more blocking oligonucleotides are used during reverse transcription and when no blocking oligonucleotides are used during reverse transcription is at most 20%, at most 15%, at most 10%, at most 8%, at most 6%, or at most 5% of the Ct value when no blocking oligonucleotides are used during reverse transcription.

The degree of off-target depletion by blocking oligonucleotides may also be analyzed via whole transcriptome sequencing. An exemplary method is disclosed in Example 2 below. Briefly, a scatter plot may be generated comparing the relative gene expression for genes other than those encoding the one or more unwanted RNA species when one or more blocking oligonucleotides are used during reverse transcription with when no blocking oligonucleotides are used during reverse transcription. R² of the scatter plot indicates how similar the relative gene expression is between the treatment with the one or more blocking oligonucleotides and no treatment. The closer R² is to 1, the less degree of off-target depletion associated with the use of the one or more blocking oligonucleotides.

In certain embodiments, R² of the scatter plot as generated above is at least 0.85, at least 0.86, at least 0.87, at least 0.88, at least 0.89, at least 0.90, or at least 0.91.

### B. Designing Blocking Oligonucleotides

In one aspect, the present disclosure provides a method for designing blocking oligonucleotides for inhibiting cDNA synthesis of one or more unwanted RNA species in an RNA sample during reverse transcription, comprising:

(a) generating multiple blocking oligonucleotides fully complementary (preferably fully complementary) to regions of the one or more unwanted RNA species,

(b) filtering unacceptable blocking oligonucleotides,

(c) generating one or more groups of blocking oligonucleotides that are complementary to multiple different (preferably evenly spaced) regions of the one or more unwanted RNA species, and

(d) optionally shuffling blocking oligonucleotides among the groups to generate new groups of blocking oligonucleotides, and selecting one or more of the new groups of blocking oligonucleotides.

The selected group of blocking oligonucleotides is effective in inhibiting cDNA synthesis of the one or more unwanted RNA species and preferably with minimal off-target depletion. Both the effectiveness on inhibition of cDNA synthesis from the one or more unwanted RNA species and off target depletion of the selected group of blocking oligonucleotides may be evaluated as described above in Section A.

Preferably, the blocking oligonucleotides each comprise one or more modified nucleotides that increase the binding between the blocking oligonucleotides and their targeted regions of unwanted RNA species. Also preferably, the blocking oligonucleotides each comprise a 3′ modification that prevents them from being extended.

The following description uses LNA oligonucleotides as exemplary blocking oligonucleotides. Blocking oligonucleotides containing other modified nucleotides as well as those without any modified nucleotides for increasing binding to regions of unwanted RNA species but of a sufficient length for stably binding to regions of unwanted RNA species may be designed similarly to be effective in depleting unwanted RNA species and preferably with little or no off-target depletion.

1. Step (a)

Step (a) of the method for designing blocking oligonucleotides provided herein is to generate multiple blocking oligonucleotides complementary (preferably fully complementary) to regions of the one or more unwanted RNA species.

In this step, one or more parameters of blocking oligonucleotides, such as the lengths of blocking oligonucleotides, predicted Tms of duplexes formed between blocking oligonucleotides and their corresponding regions of unwanted RNA species (i.e., regions of unwanted RNA species to which the blocking oligonucleotides are fully complementary), self hybridization, and off-target hybridization in the transcriptome from which the unwanted RNA species belong(s), may be characterized and scored. The scores of the one or more parameters of each blocking oligonucleotide are used to generate a final combined score. During such a process, different parameters may be weighed differently to produce the final combined score.

The algorithm for predicting Tms of duplexes formed between blocking oligonucleotides and their corresponding regions of unwanted RNA species may be based on SantaLucia, Proc. Natl. Acad. Sci. USA 95: 1460-5, 1998, and Tm measurements of LNA containing blocking oligonucleotides.

Preferably, a memetic algorithm is used to improve and select the best blocking oligonucleotides by testing different parameters. For example, the Tm of the duplexes formed between a blocking oligonucleotide and its corresponding region of an unwanted RNA species may be improved by the following four methods: (1) reduce the number of LNA nucleotides, (2) increase the number of LNA nucleotides, (3) alter LNA nucleotide pattern, and (4) alter the blocking oligonucleotide length. In such a manner, multiple small algorithms are used to test different parameters to see if changes will improve the overall core of a blocking oligonucleotide.

LNA blocking oligonucleotides may have one, more, and all of the following characteristics:

(1) Their lengths may range from 10 to 30 nucleotides, preferably 16 to 24 nucleotides, 17 to 23 nucleotides or 18 to 22 nucleotides.

(2) The number of LNAs in each LNA blocking oligonucleotide may range from 2 to 20, preferably 4 to 16, and more preferably 3 to 15.

(3) The melting temperatures of duplexes formed between LNA blocking oligonucleotides and the regions of unwanted RNA species to which the LNA blocking oligonucleotides are complementary range from 80 to 96° C., preferably 86 to 92° C.

(4) The number of LNA blocking oligonucleotides generated in step (a) is at least 100, at least 500, at least 1000, at least 2000, at least 3000, at least 4000, at least 5000, at least 6000, at least 7000, at least 8000, at least 9000, or at least 10000, and/or at most 1,000,000, at most 500,000, at most 100,000, at most 90,000, at most 80,000, at most 70,000, at most 60,000, or at most 50,000, such as from 100 to 1,000,000, from 500 to 100,000, and from 1000 to 10,000.

(5) LNA blocking oligonucleotides are likely to bind to the regions of unwanted RNA species to which the LNA blocking oligonucleotides are complementary rather than to themselves.

(6) LNA blocking oligonucleotides are likely to bind to the regions of unwanted RNA species to which the LNA blocking oligonucleotides are complementary rather than to other regions in the transcriptome to which the unwanted RNA species belong(s).

(7) The number of the different unwanted RNA species to which the LNA blocking oligonucleotides are complementary (preferably fully complementary) is at least 2, at least 3, at least 4, or at least 5, at least 10, at least 20, at least 30, at least 40, at least 50, at least 75, at least 100, at least 200, at least 300, at least 400, or at least 500, and/or at most 1,000,000, at most 500,000, at most 100,000, at most 50,000, at most 10,000, at most 9000, at most 8000, at most 7000, at most 6000, at most 5000, at most 4000, at most 3000, or at most 2000, such as from 2 to 1,000,000, from 100 to 500,000, from 500 to 100,000, and from 1000 to 10,000.

Additional descriptions of blocking oligonucleotides are provided in Section A.5. Blocking oligonucleotides above and Section C. Sets of Blocking Oligonucleotides.

2. Step (b)

Step (b) of the method for designing blocking oligonucleotides provided herein is to filter unacceptable blocking oligonucleotides. This may be done by setting a minimum final combined score for blocking oligonucleotides. Blocking oligonucleotides with final combined scores less than the minimum final combined score are deemed unacceptable and filtered out.

3. Step (c)

Step (c) of the method for designing blocking oligonucleotides provided herein is to generate one or more groups of blocking oligonucleotides that are complementary to multiple different (preferably evenly spaced) regions of the one or more unwanted RNA species.

In certain embodiments, the groups of blocking oligonucleotides target multiple regions of a single RNA species (e.g., human 5S rRNA).

In certain other embodiments, the groups of blocking oligonucleotides target a single type of multiple RNA species from multiple organisms (e.g., bacterial 5S rRNA).

In certain other embodiments, the groups of blocking oligonucleotides target multiple types of RNA species of a single organism (e.g., human rRNAs).

In certain other embodiments, the groups of blocking oligonucleotides target multiple types of RNA species of multiple organisms (e.g., bacterial rRNAs).

To inhibit cDNA synthesis of an unwanted RNA species, it is preferred that blocking oligonucleotides are spread out along the unwanted RNA species so that no region of the unwanted RNA species will be reverse transcribed into cDNA and detected in downstream analysis. A program may be used in this step to select blocking oligonucleotides with top final combined scores and pick those that spread out evenly across the unwanted RNA species.

Preferably, multiple different regions of an unwanted RNA species to which blocking oligonucleotides are complementary are evenly spaced along the unwanted RNA species. The even distribution of the different regions allows effective inhibition of cDNA synthesis of the unwanted RNA species with a minimal or reduced number of different blocking oligonucleotides.

Regions of an unwanted RNA species are evenly spaced if the longest distance between neighboring regions is at most 2.5 times, preferably at most 2 times or at most 1.5 times, the shortest distance between neighboring regions. The distance between neighboring regions is the number of nucleotides between the 3′ terminus of the upstream region (i.e., the region closer to the 5′ terminus of the unwanted RNA species) and the 5′ terminus of the downstream region (i.e., the region closer to the 3′ terminus of the unwanted RNA species). For example, if the distances between neighboring regions of an unwanted RNA species are 30, 32, 35, 37, 38, 40, 43, and 45, such regions are deemed evenly spaced because the longest distance between neighboring region is 45, which is 1.5 time of the shortest distance 30.

The distances between evenly distributed neighboring regions of an unwanted RNA species to which blocking oligonucleotides are complementary may range from 20 to 50, 25 to 50, 30 to 50, 20 to 45, 25 to 45, 30 to 45, or 31 to 43 nucleotides.

In certain embodiments, multiple different regions of an unwanted RNA species to which blocking oligonucleotides are complementary are not evenly distributed. The distance between neighboring regions may range from 0 to 100 nucleotides, such as 0 to 75 nucleotides, 0 to 50 nucleotides, 5 to 100 nucleotides, 5 to 75 nucleotides, 5 to 50 nucleotides, 5 to 40 nucleotides, 5 to 30 nucleotides, 10 to 100 nucleotides, 10 to 75 nucleotides, 10 to 50 nucleotides, 10 to 40 nucleotides, 10 to 30 nucleotides, 20 to 100 nucleotides, 20 to 75 nucleotides, 20 to 60 nucleotides, or 30 to 100 nucleotides. In general, more blocking oligonucleotides are required if neighboring regions of an unwanted RNA species to which the blocking oligonucleotides are complementary are located close to each other (e.g., at most 25, 20, 15, 10, or 5 nucleotides apart). However, the neighboring regions should not be too far apart (e.g., more than 75, 100, 125, or 150 nucleotides apart) to avoid inadequate inhibition of cDNA synthesis using the sequences between the neighboring regions of the unwanted RNA species as templates.

In certain embodiments where a large number (e.g., at least 10, at least 50, at least 100, at least 500, at least 1000, at least 2000, at least 300, at least 4000, or at least 5000) of different unwanted RNA species are to be depleted, the group may be formed by selecting blocking oligonucleotides to increase the total coverage of the targeted unwanted RNA species the most. The different unwanted RNA species may be of a single type of unwanted RNA from multiple organisms (e.g., bacterial 5S rRNA), multiple types of unwanted RNA from a single organisms (e.g., human abundant mRNAs), or multiple types of unwanted RNA from multiple organisms (e.g., bacterial rRNAs).

In some embodiments, a single blocking oligonucleotide may target unwanted RNA species from multiple organisms that are homologous to each other (e.g., 5S rRNA from certain bacterial strains). Thus, the number of the blocking oligonucleotides in a group may be less than the number of unwanted RNA species that the blocking oligonucleotides target.

A greedy algorithm may be used for maximizing coverage of a large number of different unwanted RNA species. A greedy algorithm is an algorithm that always makes a locally-optimal choice in the hope that this choice will lead to a globally-optional solution. An exemplary greedy algorithm may include first defining the blocking oligonucleotide length (“BLOCKER LENGTH”), the distance between neighboring blocking oligonucleotides (“DISTANCE”) when annealing to the unwanted RNA species, and the number of blocking oligonucleotides (“NUMBER”) to form a group, and performing the following steps:

1. Count frequencies of all kmers with K=BLOCKER LENGTH in the set of target sequences,

2. Sort kmers by frequency,

3. Add most frequent kmer to blocker set,

4. Find location of selected kmer in all target sequences,

5. Determine kmers within 0.5 to 2 DISTANCE (preferably 1 DISTANCE) downstream of kmer location and 0.2 to 1 DISTANCE (preferably 0.5 DISTANCE) upstream in each target sequence,

6. Decrement kmers within DISTANCE in frequency list, and

7. Repeat steps 2-6 until the NUMBER of blockers is reached.

An example of using such an algorithm is provided in Example 4 for designing blocking oligonucleotides to deplete bacterial 5S, 16S and 23S rRNA sequences.

Such a design algorithm is useful in selecting a blocker that increases a total coverage of target sequence the most. Because kmer frequencies are often autocorrelated, decrementing counts of adjacent kmers avoids selecting a blocker in regions already covered by a previously selected blocker. Decrementing kmer counts upstream avoids selecting blocker too close to an already selected blocker downstream. Such an algorithm is tuned to partially cover as many target sequences as possible rather than covering fewer target sequences completely.

4. Step (d)

In certain embodiments where multiple groups are generated in step (c), the method for desgining blocking oligonucleotides may further comprise shuffling blocking oligonucleotides among the groups to generate new groups of blocking oligonucleotides and selecting one or more of the new groups of blocking oligonucleotides.

Groups of blocking oligonucleotides may be scored as the average score of the blocking oligonucleotides in the group. Parameters affecting scoring include physical parameters of blocking oligonucleotides such as melting temperature of duplexes formed between blocking oligonucleotides and their corresponding regions of unwanted RNA species, lengths of blocking oligonucleotides, self-hybridization of blocking oligonucleotides, LNA patterns, numbers of LNA nucleotides in blocking oligonucleotides, and off target hybridization of blocking oligonucleotides; and group parameters such as minimal and maximum distances between neighboring blocking oligonucleotides when annealing to their corresponding regions of unwanted RNA species and cross hybridization among blocking oligonucleotides within the group.

In this step of shuffling blocking oligonucleotides among groups of blocking oligonucleotides, cross hybridization within a group of blocking oligonucleotides is minimized. For example, the number of blocking oligonucleotides that may form duplexes with each other with a high Tm (e.g., more than 65° C.) are minimized.

A program may be used to shuffle blocking oligonucleotides and test if the score of a group of blocking oligonucleotides would be increased. This process may be repeated multiple times to generate a group of blocking oligonucleotides with a highest group score. Multiple groups of blocking oligonucleotides may be generated each with a highest group score for each of a given unwanted RNA species (e.g., one group targeting human 5.8S rRNA with a highest group score and another group targeting human 18S rRNA with another highest group score) or for a given type of unwanted RNA species (e.g., one group targeting bacterial rRNAs with a highest group score and another group targeting bacterial 16S rRNAs with another highest group score).

The selected group with a highest score may have at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 different blocking oligonucleotides, and/or at most 10,000, at most 9000, at most 8000, at most 7000, at most 6000, or at most 5000 different blocking oligonucleotides, such as from 10 to 10,000 or from 100 to 5000 different blocking oligonucleotides.

In certain embodiments, multiple groups of blocking oligonucleotides are selected, such groups may be pooled together when annealing to unwanted RNA species from a RNA sample. Alternatively, they may anneal to their target unwanted RNA species separately.

5. Experimental Testing for Blocking Efficiency and Off-Target Depletion

The selected group of blocking oligonucleotides may be further tested experimentally for its blocking efficiency and/or off-target depletion. Exemplary methods for such testing are described in Section A above and in the Examples below.

### C. Sets or Compositions of Blocking Oligonucleotides

In one aspect, the present disclosure provides a set of blocking oligonucleotides for inhibiting cDNA synthesis of an unwanted RNA species. The blocking oligonucleotides are complementary (preferably fully complementary) to multiple different (preferably evenly spaced) regions of the unwanted RNA species.

The number of blocking oligonucleotides in a set may be at least 2, at least 3, at least 4, at least 5, at least 10, at least 20, at least 30, at least 40, or at least 50, and/or at most 1000, at most 900, at most 800, at most 700, at most 600, at most 500, at most 400, at most 300, or at most 200, such as from 2 to 1000, from 5 to 500, and from 10 to 300.

Preferably, the set of blocking oligonucleotides are a set of LNA blocking oligonucleotides, and may have from one to all of the following characteristics:

(1) Their lengths may range from 10 to 30 nucleotides, preferably 16 to 24 nucleotides, 17 to 23 nucleotides or 18 to 22 nucleotides.

(2) The number of LNAs in each LNA blocking oligonucleotide may range from 2 to 20, preferably 4 to 16, and more preferably 3 to 15.

(3) The melting temperatures of duplexes formed between LNA blocking oligonucleotides and the regions of unwanted RNA species to which the LNA blocking oligonucleotides are complementary range from 80 to 96° C., preferably 86 to 92° C.

(4) Depending on the length of the unwanted RNA species, the number of LNA blocking oligonucleotides is at least 2, at least 3, at least 4, at least 5, at least 10, at least 20, at least 30, at least 40, at least 50, at least 60, at least 70, or at least 80.

(5) LNA blocking oligonucleotides are likely to bind to the regions of the unwanted RNA species to which the LNA blocking oligonucleotides are complementary rather than themselves.

(6) LNA blocking oligonucleotides are likely to bind to the regions of the unwanted RNA species to which the LNA blocking oligonucleotides are complementary rather than other regions in the transcriptome to which the unwanted RNA species belongs.

(7) (a) Regions of an unwanted RNA species to which blocking oligonucleotides are complementary are evenly distributed along the unwanted RNA species, and the distances between neighboring regions may range from 20 to 50, 25 to 50, 30 to 50, 20 to 45, 25 to 45, 30 to 45, or 31 to 43 nucleotides, or
(b) Regions of an unwanted RNA species to which blocking oligonucleotides are complementary are not evenly distributed along the unwanted RNA species, and the distances between neighboring regions may range from 0 to 100 nucleotides, such as 0 to 75 nucleotides, 0 to 50 nucleotides, 5 to 100 nucleotides, 5 to 75 nucleotides, 5 to 50 nucleotides, 5 to 40 nucleotides, 5 to 30 nucleotides, 10 to 100 nucleotides, 10 to 75 nucleotides, 10 to 50 nucleotides, 10 to 40 nucleotides, 10 to 30 nucleotides, 20 to 100 nucleotides, 20 to 75 nucleotides, 20 to 60 nucleotides, or 30 to 100 nucleotides.

In a related aspect, the present disclosure provides a plurality of sets of blocking oligonucleotides for inhibiting cDNA synthesis of multiple unwanted RNA species. Each set of blocking oligonucleotides are complementary (preferably fully complementary) to multiple different (preferably evenly spaced) regions of an unwanted RNA species as described above. In certain embodiments, different sets of blocking oligonucleotides are complementary to multiple different (preferably evenly spaced) regions of different unwanted RNA species.

The number of sets may be at least 2, at least 3, at least 4, at least 5, at least 10, at least 20, at least 30, at least 40, at least 50, at least 75, at least 100, at least 200, at least 300, at least 400, or at least 500, and/or at most 10,000, at most 9000, at most 8000, at most 7000, at most 6000, at most 5000, at most 4000, at most 3000, or at most 2000, such as from 2 to 10,000, from 2 to 5000, from 2 to 1000, from 2 to 500, from 2 to 200, from 10 to 10,000, from 10 to 5000, from 10 to 1000, from 10 to 500, from 10 to 200, from 100 to 10,000, from 100 to 5000, from 100 to 1000, or from 100 to 500.

The total number of blocking oligonucleotides in the plurality of sets of blocking oligonucleotides may be at least 5, at least 10, at least 50, at least 100, at least 150, at least 200, at least 300, at least 400, at least 500, at least 600, at least 700, at least 800, at least 900, at least 1000, at least 1500, or at least 2000, at least 3000, at least 4000, at least 5000, at least 6000, at least 7000, at least 8000, at least 9000, or at least 10,000, and/or at most 100,000, at most 90,000, at most 80,000, at most 70,000, at most 60,000, or at most 50,000, such as from 2 to 100,000, from 100 to 80,000, or from 800 to 50,000.

In certain embodiments, the multiple unwanted RNA species targeted by a plurality of sets of blocking oligonucleotides belong to multiple types of RNA species from a single organism (e.g., human 5.8S rRNA, human 18S rRNA and human 28S rRNA). In certain other embodiments, the multiple unwanted RNA species are from multiple organisms. In such embodiments, the multiple unwanted RNA species may belong to a single type of RNA species (e.g., 5S rRNA from multiple bacterial strains) or multiple different types of RNA species (e.g., 5S rRNA, 16S rRNA, and 23S rRNA from multiple bacterial strains).

The number of the different unwanted RNA species to which the sets of blocking oligonucleotides are fully complementary is at least 2, at least 3, at least 4, or at least 5, at least 10, at least 20, at least 30, at least 40, at least 50, at least 75, at least 100, at least 200, at least 300, at least 400, or at least 500, and/or at most 1,000,000, at most 500,000, at most 100,000, at most 50,000, at most 10,000, at most 9000, at most 8000, at most 7000, at most 6000, at most 5000, at most 4000, at most 3000, or at most 2000, such as from 2 to 1,000,000, from 100 to 500,000, from 500 to 100,000, and from 1000 to 10,000.

In certain embodiments, multiple sets of blocking oligonucleotides are prepared, each set targeting one or more unwanted species from a single organism (e.g., human, a plant, a specific bacterial strain). Depending on what organisms are potentially present in a given sample, different sets of blocking oligonucleotides targeting unwanted species for such organisms may be combined together and used in depleting the unwanted RNA species from those organisms. The number of different organisms whose unwanted RNA species are to be depleted may be at least 2, at least 3, at least 4, at least 5, at least 10, at least 25, at least 50, and/or at most 10,000, at most 5,000, at most 1000, at most 500, or at most 100, such as 2 to 10,000, 5 to 5,000, or 10 to 1,000.

In a related aspect, the present disclosure provides a composition or mixture comprising one or more blocking oligonucleotides, a set of blocking oligonucleotides, and/or a plurality of sets of blocking oligonucleotides as described in this section and other sections (e.g., Section A). For example, the mixture may comprise a plurality of sets of oligonucleotides that target human unwanted RNA species and one or more blocking oligonucleotides that target one or more unwanted RNA species from a pathogenic bacterial strain.

### D. Kits for Depleting Unwanted RNA Species

The present disclosure also provides a kit for inhibiting cDNA synthesis of one or more unwanted DNA species in an RNA sample, comprising: (1) (a) one or more blocking oligonucleotides that are complementary (preferably fully complementary) to one or more regions of one or more unwanted RNA species in the RNA sample, or (b) a set or a plurality of sets of blocking oligonucleotides, and (2) a reverse transcriptase.

The sections above (e.g., Sections A. 5. and C) are referred to for describing the one or more blocking oligonucleotides, the set or plurality of sets of blocking oligonucleotides, and reverse transcriptases that may be included in the kit.

In certain embodiments, the kit may further comprise from one to all of the following components:

reverse transcription primers,

reaction buffer suitable for reverse transcription,

enzymes for second cDNA strand synthesis (e.g., E. Coli RNase H DNA Polymerase I, and E. coli DNA ligase),

DNA polymerase (e.g., Taq DNA polymerase, Pfu DNA polymerase, KOD DNA polymerase, hot-start DNA polymerase, Bst DNA polymerase, Bsu DNA polymerase, Tth DNA polymerase, and Pwo DNA polymerase),

DNA Ligase (e.g., E. coli DNA ligase, T4 DNA ligase, mammalian DNA ligase, and thermostable DNA ligase),

DNA polymerase for sequencing (e.g., T7 DNA polymerase, Sequenase, Sequenase version 2),

oligonucleotide primers for DNA amplification and/or sequencing, and

adaptors (single-stranded or double stranded oligonucleotides that may be ligated to single-stranded or double stranded DNA molecules).

The components of the kits are typically contained in separate vessels or compartments. However, when appropriate, some of the components may be provided as a mixture or composition. Additional descriptions of the components are provided in other sections, including the Examples, of the present disclosure.

The following examples are for illustration and are not limiting.

### EXAMPLES

The following materials and reagents were used in Examples 1-3 of the present disclosure:

Universal Human Reference RNA (UHRR) (Agilent Technologies).

193 pool of Blockers (B1-B193), sequences of which are shown in the table below.

96 pool of Blockers (B1-B193 but only odd numbered wells, i.e., B1, B3, . . . , B193).

5×BC3 RT Buffer: 5× reverse transcription buffer from Qiagen RT2 First Strand Kit

QIAseq Beads

N6 Primer: Random Hexamer ordered from IDT (standard desalting).

Forward primer 18S FP2:
(SEQ ID NO: 1)
CTCAACACGGGAAACCTCAC
Reverse primer 18S RP2:
(SEQ ID NO: 2)
CGCTCCACCAACTAAGAACG
Forward primer 18S FP1:
(SEQ ID NO: 3)
ATGGCCGTTCTTAGTTGGTG
Reverse primer 18S RP1:
(SEQ ID NO: 4)
CGCTGAGCCAGTCAGTGTAG
Forward primer 18S FP3:
(SEQ ID NO: 5)
GTAACCCGTTGAACCCCATT
Reverse primer 18S RP3:
(SEQ ID NO: 6)
CCATCCAATCGGTAGTAGCG
Forward primer 18S FP4:
(SEQ ID NO: 7)
GGCCCTGTAATTGGAATGAGTC
Reverse primer 18S RP4:
(SEQ ID NO: 8)
CCAAGATCCAACTACGAGCTT
Forward primer GAPDH FP:
(SEQ ID NO: 9)
CACTGCCACCCAGAAGACTG
Reverse primer GAPDH RP:
(SEQ ID NO: 10)
CAGCTCAGGGATGACCTTG
Forward primer ACTB FP:
(SEQ ID NO: 11)
TGCGTGACATTAAGGAGAAGC
Reverse primer ACTB RP:
(SEQ ID NO: 12)
GGAAGGAAGGCTGGAAGAGTG
Forward primer RPLP0 FP:
(SEQ ID NO: 13)
CAATGTTGCCAGTGTCTGTC
Reverse primer RPLP0 RP:
(SEQ ID NO: 14)
AGCAAGTGGGAAGGTGTAATC

2× PA-012 Master Mix: 2× master mix for qPCR that comprises a DNA polymerase from QIAGEN.

Blockers B1-B193 Sequences

| | | | | SEQ |
|---|---|---|---|---|
| | Oligo | | Sequence | ID |
| Oligonucleotide | Position | IDT_PO | Name | NO: |
| gAcAaaCcCtTgTgtCgAg | 9711 | G + AC + AAA + CC + CT + TG + TGT + CG + AG | B193 | 15 |
| aGcTgcTcTgctAcGtAcGaaa | 9660 | A + GC + TGC + TC + TGCT + AC + GT + AC + GAAA | B192 | 16 |
| GtttAgcgCcaGgttcCcc | 9610 | + GTTT + AGCG + CCA + GGTTC + CCC | B191 | 17 |
| GgccgCctctCcggCcgc | 9560 | + GGCCG + CCTCT + CCGG + CCGC | B190 | 18 |
| CcggAccCcggtCccggC | 9510 | + CCGG + ACC + CCGGT + CCCGG + C | B189 | 19 |
| cgGggcGcgtGgaggGggg | 9460 | CG + GGGC + GCGT + GGAGG + GGGG | B188 | 20 |
| cGgctAtccGaggCcaAc | 9410 | C + GGCT + ATCC + GAGG + CCA + AC | B187 | 21 |
| GcctgGgcggGatTctGact | 9360 | + GCCTG + GGCGG + GAT + TCT + GACT | B186 | 22 |
| ggTagCttcGccccAttgGct | 9310 | GG + TAG + CTTC + GCCCC + ATTG + GCT | B185 | 23 |
| AcctgCggTtcctCtcGta | 9260 | + ACCTG + CGG + TTCCT + CTC + GTA | B184 | 24 |
| TCATCAGTaGGGtaaAaCtAA | 9210 | + T + C + A + T + C + A + G + TA + G + G + GTAA + | B183 | 25 |
| | | AA + CT + A + A | | |
| cGtTcCcTattaGtgGgTga | 9160 | C + GT + TC + CC + TATTA + GTG + GG + TGA | B182 | 26 |
| aTgAtAgGaAgAgcCgAc | 9110 | A + TG + AT + AG + GA + AG + AGC + CG + AC | B181 | 27 |
| tGaacGcttGgcCgccAcaAgc | 9060 | T + GAAC + GCTT + GGC + CGCC + ACA + AGC | B180 | 28 |
| AcCtCcTgcTtAaaAcCcAaaa | 9010 | + AC + CT + CC + TGC + TT + AAA + AC + CC + AAAA | B179 | 29 |
| cGgTcTgTatTcGtacTgAa | 8960 | C + GG + TC + TG + TAT + TC + GTAC + TG + AA | B178 | 30 |
| cTccaCggGagGtttCtgT | 8910 | C + TCCA + CGG + GAG + GTTT + CTG + T | B177 | 31 |
| CgTtAccgtTtGacAgGtgtAc | 8860 | + CG + TT + ACCGT + TT + GAC + AG + GTGT + AC | B176 | 32 |
| cCcggAgcgGgtcGcgcC | 8810 | C + CCGG + AGCG + GGTC + GCGC + C | B175 | 33 |
| agAagCgagAgccCctCggG | 8760 | AG + AAG + CGAG + AGCC + CCT + CGG + G | B174 | 34 |
| AaaAcGaTcAgAgTaGtGg | 8710 | + AAA + AC + GA + TC + AG + AG + TA + GT + GG | B173 | 35 |
| CccgcCccGggcCcctcG | 8660 | + CCCGC + CCC + GGGC + CCCTC + G | B172 | 36 |
| TccCaCttatTcTaCaCctCtC | 8610 | + TCC + CA + CTTAT + TC + TA + CA + CCT + CT + C | B171 | 37 |
| aAgCtcAacaGgGtcTtCtTt | 8560 | A + AG + CTC + AACA + GG + GTC + TT + CT + TT | B170 | 38 |
| GctgTgGtTtCgCtggaTa | 8510 | + GCTG + TG + GT + TT + CG + CTGGA + TA | B169 | 39 |
| AtCcAtTcAtGcGcGtCaCtaa | 8460 | + AT + CC + AT + TC + AT + GC + GC + GT + CA + CTAA | B168 | 40 |
| GaGtCatAgTtacTcccgC | 8410 | + GA + GT + CAT + AG + TTAC + TCCCG + C | B167 | 41 |
| tTtGaCaTtCagAgCacTg | 8360 | T + TT + GA + CA + TT + CAG + AG + CAC + TG | B166 | 42 |
| cgGgcCttCgcGatGctTt | 8310 | CG + GGC + CTT + CGC + GAT + GCT + TT | B165 | 43 |
| CcgCacCagTtcTaaGtcGg | 8260 | + CCG + CAC + CAG + TTC + TAA + GTC + GG | B164 | 44 |
| cgGaaCcgcgGccccGgg | 8210 | CG + GAA + CCGCG + GCCCC + GGG | B163 | 45 |
| CccctCcgcCgcctGccgC | 8160 | + CCCCT + CCGC + CGCCT + GCCG + C | B162 | 46 |
| aaCgggGggcGgacgGggc | 8110 | AA + CGGG + GGGC + GGACG + GGGC | B161 | 47 |
| GccccGccgcCcgccGac | 8060 | + GCCCC + GCCGC + CCGCC + GAC | B160 | 48 |
| aGcggAcgcGcgCgcgAcgAga | 8010 | A + GCGG + ACGC + GCG + CGCG + ACG + AGA | B159 | 49 |
| cgccGggctCcccGggggC | 7960 | CGCC + GGGCT + CCCC + GGGGG + C | B158 | 50 |
| cAcgGgaAggGcccGgctc | 7910 | C + ACG + GGA + AGG + GCCC + GGCTC | B157 | 51 |
| gggtGcccgGgcCccCct | 7860 | GGGT + GCCCG + GGC + CCC + CCT | B156 | 52 |
| ccgcGgcggGccgCcgccG | 7810 | CCGC + GGCGG + GCCG + CCGCC + G | B155 | 53 |
| CcgcCcccaCgcgGcgC | 7760 | + CCGC + CCCCA + CGCG + GCG + C | B154 | 54 |
| gGaGaGaGaGagAgAgAg | 7710 | G + GA + GA + GA + GA + GAG + AG + AG + AG | B153 | 55 |
| cGcgggGtgggGcgGggga | 7660 | C + GCGGG + GTGGG + GCG + GGGGA | B152 | 56 |
| gGgcggCgGcgccTcgtC | 7610 | G + GGCGG + CG + GCGCC + TCGT + C | B151 | 57 |
| CcccaGcccgAccgaCcc | 7560 | + CCCCA + GCCCG + ACCGA + CCC | B150 | 58 |
| AcggaTccGgcTtgCcgAc | 7510 | + ACGGA + TCC + GGC + TTG + CCG + AC | B149 | 59 |
| GagGctGttCacCttGgaGa | 7460 | + GAG + GCT + GTT + CAC + CTT + GGA + GA | B148 | 60 |
| GagaTttaCacCctCtcCcc | 7410 | + GAGA + TTTA + CAC + CCT + CTC + CCC | B147 | 61 |
| gAcgCcgcCggaaCcgCga | 7360 | G + ACG + CCGC + CGGAA + CCG + CGA | B146 | 62 |
| cGaAcccaTtcCaGggCg | 7310 | C + GA + ACCCA + TTC + CA + GGG + CG | B145 | 63 |
| cccgGggctCccGccGgct | 7260 | CCCG + GGGCT + CCC + GCC + GGCT | B144 | 64 |
| gcCtcgcGgcGcccAtcT | 7210 | GC + CTCGC + GGC + GCCC + ATC + T | B143 | 65 |
| CcgacTccctTtcgAtcGgcCg | 7160 | + CCGAC + TCCCT + TTCG + ATC + GGC + CG | B142 | 66 |
| aAcggCgcTcgcCcatCt | 7110 | A + ACGG + CGC + TCGC + CCAT + CT | B141 | 67 |
| CtgttCacAtGgAaCcCttCt | 7060 | + CTGTT + CAC + AT + GG + AA + CC + CTT + CT | B140 | 68 |
| AttTgCtAcTaCcAcCaAg | 7010 | + ATT + TG + CT + AC + TA + CC + AC + CA + AG | B139 | 69 |
| CgCcCtaGgcTtcaAggc | 6960 | + CG + CC + CTA + GGC + TTCA + AGGC | B138 | 70 |
| TagcgTccgCgggGctCc | 6910 | + TAGCG + TCCG + CGGG + GCT + CC | B137 | 71 |
| gggaGgaggCgtGggGgg | 6860 | GGGA + GGAGG + CGT + GGG + GGG | B136 | 72 |
| cgcCgccgCcgCcgccC | 6810 | CGC + CGCCG + CCG + CCGCC + C | B135 | 73 |
| CcgccCccGccgCtcccG | 6760 | + CCGCC + CCC + GCCG + CTCCC + G | B134 | 74 |
| TggGcccGacgcTccAgcG | 6710 | + TGG + GCCC + GACGC + TCC + AGC + G | B133 | 75 |
| gCaGgTgagtTgTtAcAcActc | 6660 | G + CA + GG + TGAGT + TG + TT + AC + AC + ACTC | B132 | 76 |
| TcCtGcTgTcTaTaTcAaCc | 6610 | + TC + CT + GC + TG + TC + TA + TA + TC + AA + CC | B131 | 77 |
| AtCgggcGcCtTaAcccg | 6560 | + AT + CGGGC + GC + CT + TA + ACCCG | B130 | 78 |
| TgCtTaCcAaAaGtgGcccAc | 6510 | + TG + CT + TA + CC + AA + AA + GTG + GCCC + AC | B129 | 79 |
| ccagCgagcCggGcttCtt | 6460 | CCAG + CGAGC + CGG + GCTT + CTT | B128 | 80 |
| aTcgTttCggCcccaAgaCct | 6410 | A + TCG + TTT + CGG + CCCCA + AGA + CCT | B127 | 81 |
| TggcgGgggTgcgtCgggT | 6360 | + TGGCG + GGGG + TGCGT + CGGG + T | B126 | 82 |
| tTcggAggGaaCcAgCtAc | 6310 | T + TCGG + AGG + GAA + CC + AG + CT + AC | B125 | 83 |
| tAccCaggTcggAcgAccgaT | 6260 | T + ACC + CAGG + TCGG + ACG + ACCGA + T | B124 | 84 |
| GagTttCctCtggCttCg | 6210 | + GAG + TTT + CCT + CTGG + CTT + CG | B123 | 85 |
| GgtCctAacAcgTgcGctCg | 6160 | + GGT + CCT + AAC + ACG + TGC + GCT + CG | B122 | 86 |
| ggccGgtggTgcGccctC | 6110 | GGCC + GGTGG + TGC + GCCCT + C | B121 | 87 |
| cggcCggcgAgcGcgCcgg | 6060 | CGGC + CGGCG + AGC + GCG + CCGG | B120 | 88 |
| GtgcGagcCcccgActcgC | 6010 | + GTGC + GAGC + CCCCG + ACTCG + C | B119 | 89 |
| TcaagAcgggTcggGtgGgtAg | 5960 | + TCAAG + ACGGG + TCGG + GTG + GGT + AG | B118 | 90 |
| cgCcgTcccCctctTcgg | 5910 | CG + CCG + TCCC + CCTCT + TCGG | B117 | 91 |
| ccgGgcccGacggCgcga | 5860 | CCG + GGCCC + GACGG + CGCGA | B116 | 92 |
| cgCccCccgaCccGcgcG | 5810 | CG + CCC + CCCGA + CCC + GCGC + G | B115 | 93 |
| GggGagGagggGtgGgaG | 5760 | + GGG + GAG + GAGGG + GTG + GGA + G | B114 | 94 |
| CccccAcgagGagAcgCc | 5710 | + CCCCC + ACGAG + GAG + ACG + CC | B113 | 95 |
| gGggAttCcccgCggggG | 5660 | G + GGG + ATT + CCCCG + CGGGG + G | B112 | 96 |
| ggtcTcgctCccTcggCc | 5610 | GGTC + TCGCT + CCC + TCGG + CC | B111 | 97 |
| gGgctgTaacActcGggGggg | 5560 | G + GGCTG + TAAC + ACTC + GGG + GGGG | B110 | 98 |
| CaccgCcgcCgccgCcgcC | 5510 | + CACCG + CCGC + CGCCG + CCGC + C | B109 | 99 |
| AcgcGgggCcgGgggGcgga | 5460 | + ACGC + GGGG + CCG + GGGG + GCGGA | B108 | 100 |
| gaCggggCcccCcgaGcc | 5410 | GA + CGGGG + CCCC + CCGA + GCC | B107 | 101 |
| ggAgccGgtcgCggcGcac | 5360 | GG + AGCC + GGTCG + CGGC + GCAC | B106 | 102 |
| GtcGccggTcgGgggAcg | 5310 | + GTC + GCCGG + TCG + GGGG + ACG | B105 | 103 |
| gCccaCccCcgcaCccGc | 5260 | G + CCCA + CCC + CCGCA + CCC + GC | B104 | 104 |
| agGaggAggAggGgcggC | 5221 | AG + GAGG + AGG + AGG + GGCGG + C | B103 | 105 |
| GgaGgaacGgggGgcGggaaAg | 5170 | + GGA + GGAAC + GGGG + GGC + GGGAA + AG | B102 | 106 |
| gCcggGttGaatcCtcCg | 5119 | G + CCGG + GTT + GAATC + CTC + CG | B101 | 107 |
| CtcTtAacgGtttCaCgCcCtc | 5068 | + CTC + TT + AACG + GTTT + CA + CG + CC + CTC | B100 | 108 |
| tCcCtTaCggTaCttGtTg | 5017 | T + CC + CT + TA + CGG + TA + CTT + GT + TG | B99 | 109 |
| tAgAtgGaGttTaCcAcccGct | 4966 | T + AG + ATG + GA + GTT + TA + CC + ACCC + GCT | B98 | 110 |
| aaGacCcgggCccggCgc | 4915 | AA + GAC + CCGGG + CCCGG + CGC | B97 | 111 |
| gGgcTgggCctCgaTcag | 4864 | G + GGC + TGGG + CCT + CGA + TCAG | B96 | 112 |
| agCggGtcTtccGtacGc | 4813 | AG + CGG + GTC + TTCC + GTAC + GC | B95 | 113 |
| TtcggCgcTgggcTctTcc | 4762 | + TTCGG + CGC + TGGGC + TCT + TCC | B94 | 114 |
| gTtaGtTtCtTctCctccGc | 4711 | G + TTA + GT + TT + CT + TCT + CCTCC + GC | B93 | 115 |
| gTctGatCtgAgGtcgCg | 4660 | G + TCT + GAT + CTG + AG + GTCG + CG | B92 | 116 |
| CtTtTactTcCtcTaGaTaGt | 4596 | + CT + TT + TACT + TC + CTC + TA + GA + TA + GT | B91 | 117 |
| GccgTgggcCgaCcccgG | 4545 | + GCCG + TGGGC + CGA + CCCCG + G | B90 | 118 |
| TccAatcGgTaGtAgCgacGg | 4494 | + TCC + AATC + GG + TA + GT + AG + CGAC + GG | B89 | 119 |
| AaCgCaAgcTtAtgAcccGca | 4443 | + AA + CG + CA + AGC + TT + ATG + ACCC + GCA | B88 | 120 |
| aTtgCaaTccCcgAtccCca | 4392 | A + TTG + CAA + TCC + CCG + ATCC + CCA | B87 | 121 |
| TgccGgcGtagGgtAggca | 4341 | + TGCC + GGC + GTAG + GGT + AGGCA | B86 | 122 |
| GcaGccccGgacAtcTaaggGc | 4290 | + GCA + GCCCC + GGAC + ATC + TAAGG + GC | B85 | 123 |
| cTgaAcgcCacTtgTccc | 4239 | C + TGA + ACGC + CAC + TTG + TCCC | B84 | 124 |
| GgGgTcGcgtaActAgttAgc | 4188 | + GG + GG + TC + GCGTA + ACT + AGTT + AGC | B83 | 125 |
| cCaGacAaAtCgCtccAcca | 4137 | C + CA + GAC + AA + AT + CG + CTCC + ACCA | B82 | 126 |
| GgAaTcGaGaAaGaGcTaTcaa | 4086 | + GG + AA + TC + GA + GA + AA + GA + GC + TA + TCAA | B81 | 127 |
| GtgaGgTtTcccgTgttgAgtc | 4035 | + GTGA + GG + TT + TCCCG + TGTTG + AGTC | B80 | 128 |
| cCtTccgTcaaTtcCtTt | 3984 | C + CT + TCCG + TCAA + TTC + CT + TT | B79 | 129 |
| GgAaCcCaAagAcTtTggTtt | 3933 | + GG + AA + CC + CA + AAG + AC + TT + TGG + TTT | B78 | 130 |
| gccgCcgcaTcgCcggTcg | 3881 | GCCG + CCGCA + TCG + CCGG + TCG | B77 | 131 |
| TcTgAtCgTcTtcgAaCctCc | 3830 | + TC + TG + AT + CG + TC + TTCG + AA + CCT + CC | B76 | 132 |
| GgcAaAtGcTtTcGcTcTg | 3779 | + GGC + AA + AT + GC + TT + TC + GC + TC + TG | B75 | 133 |
| tCtAgcGgCgCaAtacGaat | 3728 | T + CT + AGC + GG + CG + CA + ATAC + GAAT | B74 | 134 |
| aGttCcGaAaAcCaacAaAa | 3677 | A + GTT + CC + GA + AA + AC + CAAC + AA + AA | B73 | 135 |
| CtgcgGtaTccagGcggCtc | 3626 | + CTGCG + GTA + TCCAG + GCGG + CTC | B72 | 136 |
| agTaaacGctTcgggCccCg | 3575 | AG + TAAAC + GCT + TCGGG + CCC + CG | B71 | 137 |
| cGagAggcAagGggCggg | 3524 | C + GAG + AGGC + AAG + GGG + CGGG | B70 | 138 |
| cGcccGcTcccAaGatcc | 3473 | C + GCCC + GC + TCCC + AA + GATCC | B69 | 139 |
| tAtAcGcTatTgGagCtGg | 3422 | T + AT + AC + GC + TAT + TG + GAG + CT + GG | B68 | 140 |
| cCtCcaAtggAtCctCgTtAa | 3371 | C + CT + CCA + ATGG + AT + CCT + CG + TT + AA | B67 | 141 |
| gCctCgaAaGagTcCtGta | 3320 | G + CCT + CGA + AA + GAG + TC + CT + GTA | B66 | 142 |
| tCgGgagTggGtaatTtGcGcg | 3269 | T + CG + GGAG + TGG + GTAAT + TT + GC + GCG | B65 | 143 |
| TctcaGgcTccctCtccGga | 3218 | + TCTCA + GGC + TCCCT + CTCC + GGA | B64 | 144 |
| AccAtgGtaGgcAcgGcgAc | 3167 | + ACC + ATG + GTA + GGC + ACG + GCG + AC | B63 | 145 |
| tgGgtcgTcgCcgcCacg | 3116 | TG + GGTCG + TCG + CCGC + CACG | B62 | 146 |
| GagtcAccAaagcCgcCggcg | 3065 | + GAGTC + ACC + AAAGC + CGC + CGGCG | B61 | 147 |
| GacCggGttGgtTttGatCt | 3014 | + GAC + CGG + GTT + GGT + TTT + GAT + CT | B60 | 148 |
| cAgcGcccgTcggCatgT | 2963 | C + AGC + GCCCG + TCGG + CATG + T | B59 | 149 |
| GtaGgagAggAgcGagcgAcc | 2912 | + GTA + GGAG + AGG + AGC + GAGCG + ACC | B58 | 150 |
| cGcaGtTtcAcTgTaCcGgc | 2861 | C + GCA + GT + TTC + AC + TG + TA + CC + GGC | B57 | 151 |
| CtTtgAgaCaAgCaTaTgCtAc | 2810 | + CT + TTG + AGA + CA + AG + CA + TA + TG + CT + AC | B56 | 152 |
| gAcAgGcGtaGccccGggaG | 2759 | G + AC + AG + GC + GTA + GCCCC + GGGA + G | B55 | 153 |
| gTcGaTgAtcAaTgTgTcctGc | 2708 | G + TC + GA + TG + ATC + AA + TG + TG + TCCT + GC | B54 | 154 |
| tCttCatCgacgCacGagCc | 2657 | T + CTT + CAT + CGACG + CAC + GAG + CC | B53 | 155 |
| cTtgGgtGggtgTggGta | 2606 | C + TTG + GGT + GGGTG + TGG + GTA | B52 | 156 |
| GgaaGgCgcTtTgTgaAgt | 2555 | + GGAA + GG + CGC + TT + TG + TGA + AGT | B51 | 157 |
| GgGagGaaTtTgAaGtAgAtAg | 2504 | + GG + GAG + GAA + TT + TG + AA + GT + AG + AT + AG | B50 | 158 |
| TcAgAtCaCgTaGgAcTtTaat | 2453 | + TC + AG + AT + CA + CG + TA + GG + AC + TT + TAAT | B49 | 159 |
| cCaTcGgGaTgtCctgAt | 2402 | C + CA + TC + GG + GA + TGT + CCTG + AT | B48 | 160 |
| AtGgAcTcTaGaAtAgGat | 2351 | + AT + GG + AC + TC + TA + GA + AT + AG + GAT | B47 | 161 |
| gTtgGtCaaGtTaTtGgAtCa | 2300 | G + TTG + GT + CAA + GT + TA + TT + GG + AT + CA | B46 | 162 |
| GaAgTctTaGcAtGtacTgcTc | 2249 | + GA + AG + TCT + TA + GC + AT + GTAC + TGC + TC | B45 | 163 |
| CcGaAATTTttaAtGcAGg | 2198 | + CC + GA + A + A + T + T + TTTA + AT + GC + A + GG | B44 | 164 |
| GGTACTGTTTGcaTtaAtAAa | 2147 | + G + G + T + A + C + T + G + T + T + T + GCA + | B43 | 165 |
| | | TTA + AT + A + AA | | |
| tgTgtTatGccCgcCtcTtcA | 2096 | TG + TGT + TAT + GCC + CGC + CTC + TTC + A | B42 | 166 |
| GaCagctGaAcCcTcgTg | 2045 | + GA + CAGCT + GA + AC + CC + TCG + TG | B41 | 167 |
| CaAgTgAtTaTgCtAcCtTt | 1994 | + CA + AG + TG + AT + TA + TG + CT + AC + CT + TT | B40 | 168 |
| tgTgtCactGggcaGgcgGtg | 1943 | TG + TGT + CACT + GGGCA + GGCG + GTG | B39 | 169 |
| gTttTtGgTaaAcagGcgGgGt | 1892 | G + TTT + TT + GG + TAA + ACAG + GCG + GG + GT | B38 | 170 |
| AcCtTtcctTaTgAgCatGc | 1841 | + AC + CT + TTCCT + TA + TG + AG + CAT + GC | B37 | 171 |
| TgAcTtGtTgGtTgAtTgTaga | 1790 | + TG + AC + TT + GT + TG + GT + TG + AT + TG + TAGA | B36 | 172 |
| AatCtGaCgCaGgCtTaTg | 1739 | + AAT + CT + GA + CG + CA + GG + CT + TA + TG | B35 | 173 |
| AACATTAGttcTtCTATaGg | 1688 | + A + A + C + A + T + T + A + GTTC + TT + C + T + | B34 | 174 |
| | | A + TA + GG | | |
| AgTtcAgtTaTaTgTtTgGgAt | 1637 | + AG + TTC + AGT + TA + TA + TG + TT + TG + GG + AT | B33 | 175 |
| GctTtctTaaTtggTggCtgCt | 1586 | + GCT + TTCT + TAA + TTGG + TGG + CTG + CT | B32 | 176 |
| AcTcTcTcTaCaAggTtttTt | 1535 | + AC + TC + TC + TC + TA + CA + AGG + TTTT + TT | B31 | 177 |
| GACtaAcaGTTaaAtTtAcAag | 1484 | + G + A + CTA + ACA + G + T + TAA + AT + TT + AC + | B30 | 178 |

AAG

| | | | | |
|---|---|---|---|---|
| GTTgAActaAgatTCtaTc | 1433 | + G + T + TG + A + ACTA + AGAT + T + CTA + TC | B29 | 179 |
| GttTgtCgcCtcTacCtaTa | 1382 | + GTT + TGT + CGC + CTC + TAC + CTA + TA | B28 | 180 |
| GgTgtGctCtTtTaGcTgTtCt | 1331 | + GG + TGT + GCT + CT + TT + TA + GC + TG + TT + CT | B27 | 181 |
| tTggCtCtCctTgCaaag | 1280 | T + TGG + CT + CT + CCT + TG + CAAAG | B26 | 182 |
| aTaggGgTtagTcctTgCtA | 1229 | A + TAGG + GG + TTAG + TCCT + TG + CT + A | B25 | 183 |
| cCtTgCgGtAcTaTaTctAt | 1178 | C + CT + TG + CG + GT + AC + TA + TA + TCT + AT | B24 | 184 |
| ACTTTaTTtGggTaaaTggtTt | 1127 | + A + C + T + T + TA + T + TT + GGG + TAAA +T | B23 | 185 |
| | | GGT + TT | | |
| tGggtTtggGgcTaggTttAgc | 1076 | T + GGGT + TTGG + GGC + TAGG + TTT + AGC | B22 | 186 |
| tTaCgAcTtGtcTcCtcTa | 1021 | T + TA + CG + AC + TT + GTC + TC + CTC + TA | B21 | 187 |
| TcCtTtGaAgTaTaCtTgAgga | 970 | + TC + CT + TT + GA + AG + TA + TA + CT + TG + AGGA | B20 | 188 |
| cCcTgTtCaAcTaAgCaCtC | 919 | C + CC + TG + TT + CA + AC + TA + AG + CA + CT + C | B19 | 189 |
| cGaCcCtTaAgTtTcAtaaGgg | 868 | C + GA + CC + CT + TA + AG + TT + TC + ATAA + GGG | B18 | 190 |
| ccAtttCtTgCcAcCtcAt | 817 | CC + ATTT + CT + TG + CC + AC + CTC + AT | B17 | 191 |
| GtAcTtGcGcTtAcTtTgt | 766 | + GT + AC + TT + GC + GC + TT + AC + TT + TGT | B16 | 192 |
| gGtAtaTaggcTgAgCaAgAgg | 715 | G + GT + ATA + TAGGC + TG + AG + CA + AG + AGG | B15 | 193 |
| GaacAggcTccTctaGaggg | 664 | + GAAC + AGGC + TCC + TCTA + GAGGG | B14 | 194 |
| agCtgTggcTcgTagtgTt | 613 | AG + CTG + TGGC + TCG + TAGTG + TT | B13 | 195 |
| gAggTttAgGgCtAaGcatAg | 562 | G + AGG + TTT + AG + GG + CT + AA + GCAT + AG | B12 | 196 |
| GcTatTgtGtGtTcAgAtAtGt | 511 | + GC + TAT + TGT + GT + GT + TC + AG + AT + AT + GT | B11 | 197 |
| CaAcTgGaGtTtTtTaCaActc | 460 | + CA + AC + TG + GA + GT + TT + TT + TA + CA + ACTC | B10 | 198 |
| AcacTctTtacGccGgctTc | 401 | + ACAC + TCT + TTAC + GCC + GGCT + TC | B9 | 199 |
| gGtgGcTgGcAcgAaaTtgAcc | 351 | G + GTG + GC + TG + GC + ACG + AAA + TTG + ACC | B8 | 200 |
| AcTtTcGtTtAtTgCtAaAggt | 301 | + AC + TT + TC + GT + TT + AT + TG + CT + AA + AGGT | B7 | 201 |
| GctAggcTaAgCgTtTtgaGc | 251 | + GCT + AGGC + TA + AG + CG + TT + TTGA + GC | B6 | 202 |
| CtTttGatCgTgGtGaTtTaGa | 201 | + CT + TTT + GAT + CG + TG + GT + GA + TT + TA + GA | B5 | 203 |
| gTgTaAtCtTaCtaAgAg | 151 | G + TG + TA + AT + CT + TA + CTA + AG + AG | B4 | 204 |
| AgcCtaCagcAcccGgtat | 101 | + AGC + CTA + CAGC + ACCC + GGTAT | B3 | 205 |
| gGcccgAcccTgcttAgc | 51 | G + GCCCG + ACCC + TGCTT + AGC | B2 | 206 |
| GgtgGtatGgCcGtaGac | 1 | + GGTG + GTAT + GG + CC + GTA + GAC | B1 | 207 |

### Example 1

### Comparison Between Exemplary Method of Present Disclosure with Ribo-Zero rRNA Removal Kit

This Example describes unwanted RNA depletion of an exemplary method of the present disclosure with that using the Ribo-Zero rRNA

Removal kit by Illumina via qPCR.

Step by Step Workflow:

1a. Hybridize blockers to total RNA sample
A. Mix 100 ng of Universal Human Reference RNA (UHRR) (Agilent Technologies) with blockers (B1 to B193) in a volume of 15 ul that also contains 20 mM KCl.
B. Incubate in thermocycler:

| Temp. | Time |
|---|---|
| 75° C. | 2 min |
| 70° C. | 2 min |
| 65° C. | 2 min |
| 60° C. | 2 min |
| 55° C. | 2 min |
| 37° C. | 5 min |
| 25° C. | 5 min |
| 4° C. | Hold |

1b. rRNA depletion using Illumina Ribo-zero rRNA Removal kit:
A. For each reaction, wash 225 ul magnetic beads with 225 ul water twice. Remove all supernatant.
B. Add 65 ul magnetic beads Resuspension Solution and mix. Set aside at room temperature.
C. In another tube mix 10 ul of Ribo-zero Removal Solution, 4 ul reaction buffer, RNA sample, and water, to total volume of 40 ul. Incubate at 68° C. for 10 min. Incubate at room temperature for 5 min.
D. Mix sample from step C with sample from step B, incubate at room temperature for 5 min. Incubate at 50° C. 5 min.
E. Transfer supernatant (i.e., depleted sample) to clean tube.
F. Add 2 volumes of QIAseq beads to 1 volume of sample from step E. After RNA is bound, wash with 200 ul 80% ethanol twice. Dry. Elute final sample in 20 ul water.

2a. Reverse transcription reaction after step 1a
A. Mix together
   RNA from previous step: 13 ul
   5×BC3 Buffer: 4 ul
   1 mM N6 Primer: 1 ul
   RNase Inhibitor (40 U/ul): 1 ul
   ENZScript (200 U/ul MMLV Reverse Transcriptase RNase H−): 1 ul
   Total Volume: 20 ul
B. Incubate in thermocycler: 25° C. 10 min, 42° C. 30 min, 4° C. hold.

2b. Reverse transcription reaction after step 1b
Performs the same as in step 2a with one exception: Instead of using 13 ul of sample, only use 0.36 ul (to achieve equivalent input as in step 2a)

3. Purify cDNA
Add 80 ul water and 130 ul QIAseq beads to 20 ul sample from step 2a or step 2b. Wash bound cDNA with 200 ul 80% ethanol (EtOH) twice. Dry. Elute in 20 ul water.

4. Perform qPCR
A. Mix together
   cDNA from previous step: 2 ul
   5 uM forward primer: 0.8 ul
   5 uM reverse primer: 0.8 ul
   2×PA-012 Master Mix: 5 ul
   Total Volume: 10 ul
B. Incubate in real-time instrument: 95° C. 9 min, 98° C. 1 min, 40 cycles of (98° C. 15 sec, 60° C. 1.5 min with data collection).

qPCR Data

| | | | qPCR (Input is 10 ng equiv.) | | | | | | |
|---|---|---|---|---|---|---|---|---|---|
| | | Blockers | Ct (18S | Ct (18S | Ct (18S | Ct (18S | Ct | Ct | Ct |
| | | B1-B193. | FP2 & | FP1 & | FP3 & | FP4 & | (GAPDH | (ACTB | (RPLP0 |
| | | pmol | RP2 | RP1 | RP3 | RP4 | FP & RP | FP & RP | FP & RP |
| Sample | Input | (each) | Primers) | Primers) | Primers) | Primers) | Primers) | Primers) | Primers) |
| 1 | 100 ng UHRR | 18.55 | 29.6 | 33.3 | 35.9 | 40 | 17.3 | 17.5 | 18.9 |
| 2 | 100 ng UHRR | 8.75 | 22.8 | 24.8 | 27.7 | 28.6 | 15.4 | 15.3 | 17 |
| 3 | 100 ng UHRR | 3.5 | 13.3 | 19.7 | 20.5 | 18.5 | 15.1 | 15.8 | 16.6 |
| 4 | 100 ng UHRR | 1.4 | 8.4 | 9.6 | 10.9 | 10 | 15 | 15.9 | 16 |
| 5 | 100 ng UHRR | 0.56 | 6 | 6.5 | 8.9 | 7.3 | 14.9 | 15.4 | 15.6 |
| 6 | 100 ng UHRR | None | 4.8 | 5.1 | 7.5 | 5.1 | 15 | 15.1 | 15.5 |
| 7 | 5 ug UHRR Ribo- | N/A | 22 | 23.2 | 23.7 | 22.8 | 15.6 | 17 | 16 |
| | Zero Depleted | | | | | | | | |
| 8 | 5 ug UHRR No | N/A | 4.8 | 5.0 | 7.1 | 5.6 | 14.7 | 15.9 | 16.3 |
| | Ribo-Zero | | | | | | | | |

Summary of Data:

Ct values of samples 1-5 show that using increasing amount of B1-6193 blockers resulted in less synthesis of the 18S rRNA cDNA region measured by the 4 qPCR primer assays (18S FP2 and RP2, 18S FP1 and RP1, 18S FP3 and RP3, and 18S FP4 and RP4) compared with those of sample 6 without any blockers. Using 18.55 pmol of each blocker gave the best results in blocking the synthesis of 18S rDNA cDNA synthesis.

Ct values for the 3 house-keeping genes (GAPDH, ACTB and RPLP0) of samples 2-5 indicate that there were no off-target effects due to the presence of blockers because of similar Ct values of samples 2-5 compared to sample 6 without any blockers. 18.55 pmol each blocker (sample 1) caused additional off-target effects compared to no blockers (sample 6).

Comparisons of Ct values between sample 7 (Ribo-Zero depleted) and sample 8 (no Ribo-Zero depletion) show that using Ribo-Zero rRNA Removal kit resulted in less synthesis of the 18S rRNA cDNA region measured by the qPCR primer assays, and that the Ribo-Zero depletion did not cause off-target effects.

The data further show that 8.75 pmol each of 193 blocker pool worked at least as equally well as Ribo-Zero in both reducing amount of rRNA cDNA and in off-target effects.

### Example 2

### Comparison of Exemplary Method of Present Disclosure with Ribo-Zero Kit, Poly(A) mRNA Enrichment and No Treatment Via Sequencing of Whole Transcriptome Libraries

This Example compared 18S rRNA depletion of an exemplary method of the present disclosure with those using the RiboZero kit, poly(A) mRNA enrichment, and no treatment via sequencing of whole transcriptome libraries.

Step by Step Workflow:

1. A. For 193 pool of Blockers: Mix together 100 ng UHRR with 8.75 pmol of each blocker. Proceed with QIAseq stranded Total RNA Library Kit in step 2 below.
B. For Illumina Ribo-zero: Use the same protocol as in step 1b of Example 1 except with the following modifications:
   Use 90 ul magnetic beads and 35 ul of Resuspension solution.
   Mix 100 ng UHRR with 2 ul Ribo-zero removal solution, 2 ul reaction buffer, and water, for a 20 ul final volume.
   Proceed with QIAseq stranded Total RNA Library Kit in step 2 below.
C. For Poly(A) mRNA enrichment: Use QIAseq stranded mRNA select kit as follows:
   i. Mix together 100 ng UHRR, 1 ul RNase inhibitor, 250 ul Buffer mRBB, 25 ul pure mRNA beads, and water to a total volume of 526 ul. Incubate at 70 C for 3 min.
   ii. Incubate at room temp for 10 min. Place on magnetic stand and remove supernatant.
   iii. Wash beads with 400 ul Buffer OW2 twice. Remove supernatant.
   iv. Add 50 ul buffer OEB, mix, incubate at 70 C for 3 min. Then incubate ate room temp for 5 min.
   v. Add 50 ul buffer mRBB and mix. Incubate at room temp for 10 min.
   vi. Pellet beads on magnetic stand then remove supernatant. Wash beads once with 400 ul buffer OW2.
   vii. Add 31 ul buffer OEB that has been heated to 70 C and mix. Pellet the beads on magnetic stand.
   viii. Take 29 ul (this contains the mRNA).
   ix. Proceed with QIAseq stranded Total RNA Library Kit in step 2 below.
D. No treatment: Mix together 100 ng UHRR and water for a total volume of 29 ul. Proceed with QIAseq stranded Total RNA Library Kit in step 2 below.

2. QIAseq Stranded Total RNA Library Kit:
Every component listed below is taken from this kit.
RNA fragmentation and Reverse-Transcription:
   i. Take sample from step 1. A, 1. B, 1. C, and 1.D, and add 8 ul of 5×RT buffer, and water, to a total volume of 37 ul.
   ii. For sample from step 1. A., fragment RNA and hybridize blockers by incubating at 95° C. 15 min then immediately ramping down to 75° C. and carry out annealing program described in Example 1. Go to step iii.
For samples 1. B., 1. C., and 1.D., fragment RNA by incubating at 95° C. 15 min, 4° C. hold. Go to step iii.
   iii. Add 1 ul RT Enzyme, 1 ul RNase Inhibitor, 1 ul of 0.4M DTT. Incubate at 25° C. 10 min, 42° C. 15 min, 70° C. 15 min, 4° C. hold.
   iv. After reverse transcription, add 56 ul QIAseq beads and mix. After cDNA is bound to beads, wash twice with 200 ul 80% EtOH. After drying beads, elute with 38.5 ul water.
Second-strand Synthesis/End-Repair/A-addition:
   v. Mix 38.5 ul sample with 5 ul Second Strand Buffer and 6.5 ul Second Strand Enzyme Mix. Incubate 25° C. 30 min, 65° C. 15 min, 4° C. hold.
   vi. Add 70 ul QIAseq beads and mix. After DNA has bound to beads, wash twice with 200 ul 80% EtOH. After beads are dry, elute with 50 ul water.
Adapter Ligation:
   vii. Dilute adapter 1:100, then add 2 ul of adapter to 50 ul sample. Add 25 ul 4× Ultralow Input Ligation Buffer, 5 ul Ultralow Input Ligase, 6.5 ul Ligation Initiator, 11.5 ul water, for a total volume of 100 ul. Mix and then incubate at 25 C for 10 min.
   viii. Add 80 ul QIAseq beads and mix. After DNA has bound to beads, wash twice with 200 ul 80% EtOH. After beads have dried, elute with 90 ul water. Add 108 ul beads to 90 ul sample and mix. After DNA has bound to beads, wash twice with 200 ul 80% EtOH. After beads have dried, elute with 23.5 ul water.
Universal PCR Amplification:
   iv. To the 23.5 ul sample add 1.5 ul CleanStart PCR Primer Mix for Illumina, and 25 ul CleanStart PCR Mix 2×, for a total volume of 50 ul.
   x. Incubate at 37° C. 15 min, 98° C. 2 min, 15 cycles of (98° C. 20 sec, 60° C. 30 sec, 72° C. 30 sec), 72° C. 1 min, 4 C hold.
   xi. Add 60 ul QIAseq beads and mix. After DNA has bound to beads, wash twice with 200 ul 80% EtOH. After beads have dried, elute with 22 ul water.
   xii. 22 ul sample is the final library ready for sequencing on Illumina NextSeq 500 system.

Sequencing Parameters:

Illumina NextSeq 500 system with 150 cycles (75×2 paired end) high-output v2. Load 1.4 pM library.

Analysis was done using Galaxy (http://usegalaxy.org). Alignment of paired-end reads using HISAT2 alignment program (Galaxy Version 2.1.0), to reference genome b37 hg19. Gene counting done with featureCounts counting program (Galaxy Version 1.6.0.2), with reference genome b37 hg19 and rRNA gtf file obtained from UCSC table browser.

### Sequencing Results

| | | Reads | Reads | | |
|---|---|---|---|---|---|
| | | aligned | aligned | Reads | % of |
| | | concord- | concord- | aligned | total |
| | | antly | antly > | concord- | reads |
| | Total | exactly 1 | 1 | antly | that is |
| Library | Reads | time | times | 0 times | rRNA |
| Blockers | 39,642,509 | 81.6% | 5.5% | 12.9% | 0.75% |
| Ribo-zero | 41,684,037 | 79.5% | 5.8% | 14.6% | 2.70% |
| Poly(A) | 40,526,691 | 80.8% | 4.6% | 14.6% | 0.14% |
| enrichment | | | | | |
| No-treatment | 36,386,107 | 37% | 48.6% | 14.4% | 63% |

Summary of Sequencing Results:

Examination of % of total reads that are rRNA reveal that the Blockers 193 pool out-performed Ribo-zero.

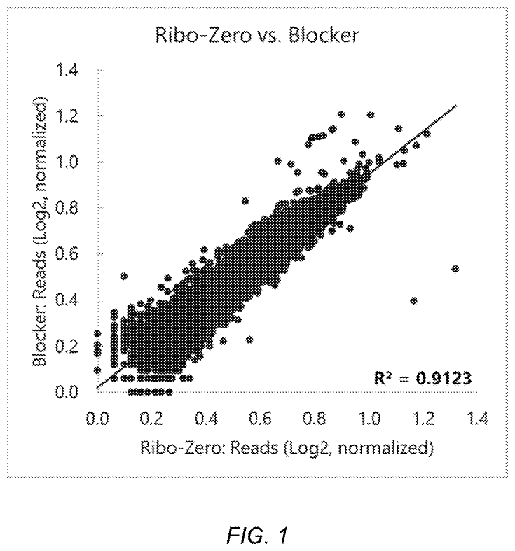

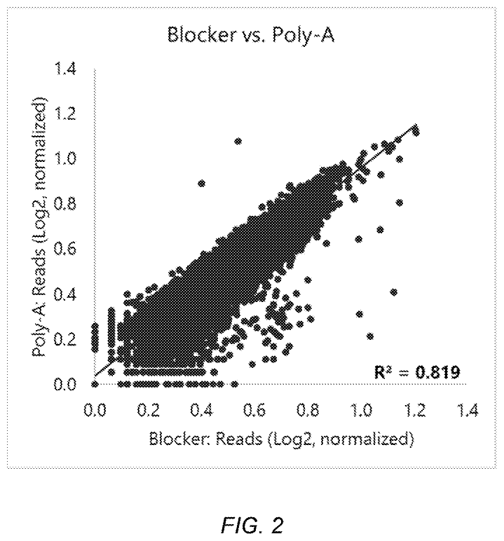

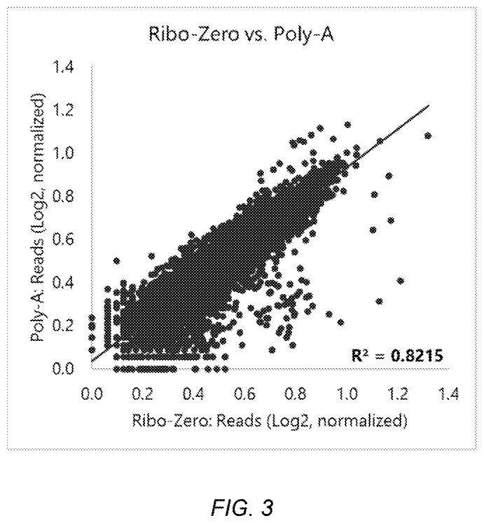

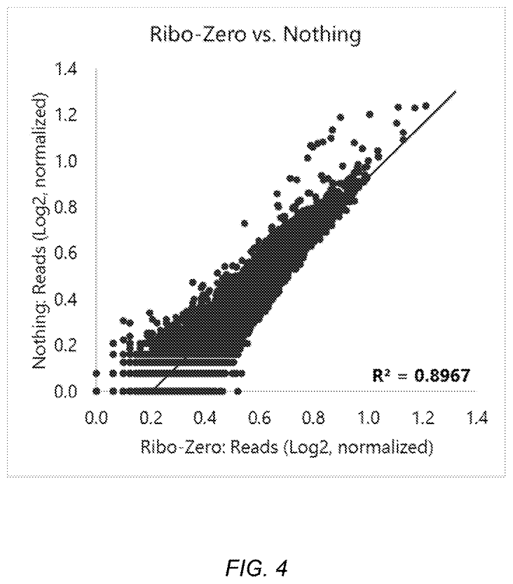

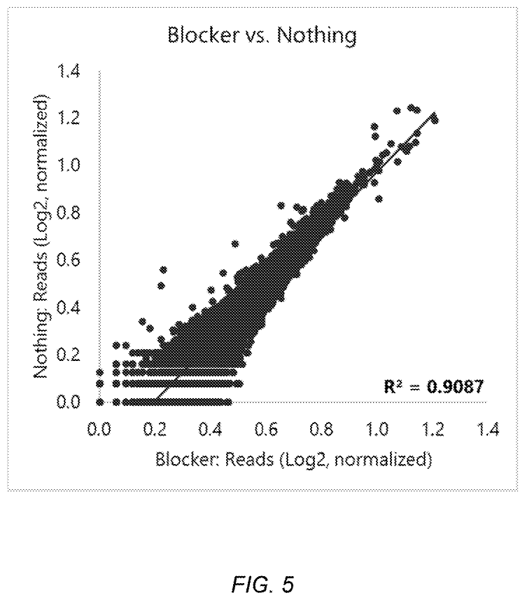

Scatter plots (FIGS. 1-5) compare the relative gene expression for non-rRNA genes of each method. Each dot represents the log 2 of the reads for each unique non-rRNA gene normalized to the average of two house-keeping genes GAPDH and ACTB. There are 16,000 genes in each scatter plot. Examination of the scatter plots reveal that both the Blockers and Ribo-zero produce similar gene expression profiles (FIG. 1, R²=0.9123), thus the blocking method did not alter gene expression profiles beyond what Ribo-zero did. In fact, the blocking method showed a slight improvement over Ribo-zero in similarity of gene expression profile of non-rRNA genes compared to No-Treatment (compare FIGS. 4 and 5). Low correlation between ribo-depletion or no-treatment and poly(A) enrichment is expected (FIGS. 2 and 3).

### Example 3

### Performance of Blockers at Different RNA Amounts

This Example tested performance of blockers at different RNA amounts.

Step by Step Workflow:

The workflow included the same steps as in Example 2 except adjusting for different input amounts, different blocker pools, different adapter dilutions, and cycles of PCR amplification (see qPCR data table below for the specifics of these changes that occurred in the QIAseq stranded RNA library kit protocol as described in Example 2). Duplicates were performed for each condition.

### qPCR Data

| | | | | | | | | | | | QIAseq stranded | |
|---|---|---|---|---|---|---|---|---|---|---|---|---|
| | Starting | | Amount | | qPCR input is 7% of starting input | | | | | | RNA Library Kit | |
| | Input | | of each | Blocker | Ct 18S | Ct 18S | Ct 18S | Ct | Ct | Ct | Adapter | Cycles of |
| Sample | (UHRR) | | Blocker | Pool | FP2/RP2 | FP1/RP1 | FP3/RP3 | GAPDH | ACTB | RPLP0 | Diln. | PCR Amp |
| 1 | 5 | ng | 8.75 pmol | 193 | 31.1 | 30.1 | 31.1 | 27.2 | 27.9 | 29.2 | 1:1000 | 21 |
| 2 | 5 | ng | 8.75 pmol | 193 | | | | | | | 1:1000 | 21 |
| 3 | 5 | ng | 4.38 pmol | 193 | 31.5 | 29 | 30.3 | 26.3 | 28.5 | 28.7 | 1:1000 | 21 |
| 4 | 5 | ng | 4.38 pmol | 193 | | | | | | | 1:1000 | 21 |
| 5 | 5 | ng | None | | 20.9 | 20.4 | 21.3 | 30.8 | 31.9 | 32.5 | 1:1000 | 21 |
| 6 | 5 | ng | None | | | | | | | | 1:1000 | 21 |
| 7 | 25 | ng | 8.75 pmol | 193 | 30.7 | 29.8 | 29.5 | 25.1 | 25.5 | 27 | 1:300 | 18 |
| 8 | 25 | ng | 8.75 pmol | 193 | | | | | | | 1:300 | 18 |
| 9 | 25 | ng | 4.38 pmol | 193 | 29.5 | 28.9 | 29.2 | 24.3 | 27.3 | 28.1 | 1:300 | 18 |
| 10 | 25 | ng | 4.38 pmol | 193 | | | | | | | 1:300 | 18 |
| 11 | 25 | ng | None | | 16.6 | 15.6 | 16.4 | 26.1 | 28.5 | 26.5 | 1:300 | 18 |
| 12 | 25 | ng | None | | | | | | | | 1:300 | 18 |
| 13 | 100 | ng | 8.75 pmol | 193 | 31.1 | 29.6 | 29.1 | 23.2 | 24.1 | 25.8 | 1:100 | 15 |
| 14 | 100 | ng | 8.75 pmol | 193 | | | | | | | 1:100 | 15 |
| 15 | 100 | ng | 4.38 pmol | 193 | 29.1 | 28 | 28.7 | 22.2 | 24 | 24.7 | 1:100 | 15 |
| 16 | 100 | ng | 4.38 pmol | 193 | | | | | | | 1:100 | 15 |
| 17 | 100 | ng | None | | 12.3 | 11.1 | 12.1 | 21.8 | 22.4 | 22.3 | 1:100 | 15 |
| 18 | 100 | ng | None | | | | | | | | 1:100 | 15 |
| 19 | 500 | ng | 8.75 pmol | 193 | 28.8 | 28.1 | 28 | 20.6 | 21.3 | 23.2 | 1:25 | 12 |
| 20 | 500 | ng | 8.75 pmol | 193 | | | | | | | 1:25 | 12 |
| 21 | 500 | ng | 8.75 pmol | 96 | 26.5 | 28.6 | 24.6 | 20.1 | 22 | 22.5 | 1:25 | 12 |
| 22 | 500 | ng | 4.38 pmol | 96 | 25.2 | 25.6 | 22.2 | 19.8 | 21 | 21.1 | 1:25 | 12 |
| 23 | 500 | ng | 4.38 pmol | 193 | 27.6 | 26.8 | 26.6 | 19.8 | 20.7 | 22 | 1:25 | 12 |
| 24 | 500 | ng | 4.38 pmol | 193 | | | | | | | 1:25 | 12 |
| 25 | 500 | ng | None | | 10.4 | 8.7 | 9.3 | 18.6 | 20.1 | 19.8 | 1:25 | 12 |
| 26 | 500 | ng | None | | | | | | | | 1:25 | 12 |
| 27 | 1000 | ng | 8.75 pmol | 193 | 28.4 | 27.8 | 27.2 | 20 | 20.7 | 22.5 | 1:12.5 | 10 |
| 28 | 1000 | ng | 8.75 pmol | 193 | | | | | | | 1:12.5 | 10 |
| 29 | 1000 | ng | 8.75 pmol | 96 | 26 | 26.2 | 24.3 | 19.4 | 20 | 20.9 | 1:12.5 | 10 |
| 30 | 1000 | ng | 4.38 pmol | 96 | 24.7 | 26.3 | 22.3 | 19 | 20.7 | 21.2 | 1:12.5 | 10 |
| 31 | 1000 | ng | 4.38 pmol | 193 | 26.9 | 26.1 | 25.9 | 19.2 | 20.9 | 21.5 | 1:12.5 | 10 |
| 32 | 1000 | ng | 4.38 pmol | 193 | | | | | | | 1:12.5 | 10 |
| 33 | 1000 | ng | None | | 10.2 | 8.1 | 9.1 | 18.2 | 19.1 | 19.4 | 1:12.5 | 10 |
| 34 | 1000 | ng | None | | | | | | | | 1:12.5 | 10 |

| | Ct AVG. HKG | | |
|---|---|---|---|
| | | 8.75 | 4.38 |
| | | pmol | pmol |
| Input | | (193 | (193 |
| (ng) | None | pool) | pool) |
| 5 | 31.7 | 28.1 | 27.8 |
| 25 | 27 | 25.9 | 26.6 |
| 100 | 22.2 | 24.4 | 23.6 |
| 500 | 19.5 | 21.7 | 20.8 |
| 1000 | 18.9 | 21.1 | 20.5 |

Summary of qPCR Data:

### 5 ng Input and 25 ng Input

Blocking of rRNA with 8.75 pmol blocker (Samples 1 and 7) worked as good as with 100 ng input (Sample 13). There was only slight reduction in blocking of rRNA with 4.38 pmol (compare Sample 3 with Sample 1 and compare Sample 9 with Sample 7). For the 3 house-keeping genes (GADPH, ACTB, and RPLP0), inclusion of blockers significantly improved detection and quantification of these genes as indicated by the decreases in Ct values of Samples 1 and 3 compared with Sample 5 and in Ct values of Samples 7 and 9 compared with Sample 11.

### 500 ng and 1000 ng Input

When using the pool of 193 blockers, blocking of rRNA with 8.75 pmol blocker (Samples 19 and 27) worked as good as with 100 ng input (Sample 13). Again, there was only a slight reduction in blocking of rRNA with 4.38 pmol (compare Sample 23 with Sample 19 and compare Sample 27 with Sample 31). There was no additional negative effect on the 3 house-keeping genes (Samples 19 and 27) as compared to 100 ng input (Sample 13).

When using the pool of 96 blockers, there was more substantial negative impact on blocking of rRNA (compare Ct values of 18S rRNA assays between Samples 21 and 19, between Samples 22 and 23, between samples 29 and 27, and between Samples 30 and 31). However, there was no additional negative impact on house-keeping genes as compared to 100 ng input (compare Ct values of house-keeping gene assays between Samples 20, 21, 29 and 30 with Sample 13).

Sequencing Parameters:

Sequencing was performed using Illumina NextSeq 500 system with 150 cycles (75×2 paired end) high-output v2. Load 1.6 pM library.

Analysis was done using Galaxy (http://usegalaxy.org). Alignment of paired-end reads was performed using HISAT2 alignment program (Galaxy Version 2.1.0) to reference genome b38 hg38. Gene counting was done with featureCounts counting program (Galaxy Version 1.6.0.2) with reference genome b38 hg38 and rRNA gtf file obtained from UCSC table browser.

### Sequencing Results

| | | | Reads | | | |
|---|---|---|---|---|---|---|
| | | | aligned | Reads | Reads | % |
| | | | concord- | aligned | aligned | reads |
| | | | antly | concord- | concord- | that |
| | | Total | exactly | antly | antly | are |
| Sample | Library | Reads | 1 time | >1 times | 0 times | rRNA |
| 1 | 5 ng, 8.75 pmol, 193 pool | 5819066 | 73% | 8.6% | 18.4% | 0.36 |
| 2 | 5 ng, 8.75 pmol, 193 pool | 8480073 | 75.8% | 7.7% | 16.5% | 0.35 |
| 3 | 5 ng, 4.38 pmol, 193 pool | 9725346 | 71.3% | 10.5% | 17.7% | 1.3 |
| 4 | 5 ng, 4.38 pmol, 193 pool | 9922453 | 70.8% | 11.4% | 17.8% | 1.6 |
| 5 | 5 ng input, None | 9889081 | 22.2% | 60.2% | 17.6% | 49 |
| 6 | 5 ng input, None | 12778827 | 24.3% | 59.9% | 15.9% | 52 |
| 7 | 25 ng, 8.75 pmol, 193 pool | 8802355 | 76.2% | 7.7% | 16.2% | 0.42 |
| 8 | 25 ng, 8.75 pmol, 193 pool | 2876583 | 75.3% | 8.1% | 16.6% | 0.26 |
| 9 | 25 ng, 4.38 pmol, 193 pool | 8764027 | 70.4% | 10.4% | 19.2% | 1.8 |
| 10 | 25 ng, 4.38 pmol, 193 pool | 6844843 | 70.9% | 10.6% | 18.5% | 1.5 |
| 11 | 25 ng, None | 14582262 | 27% | 55.7% | 17.3% | 58 |
| 12 | 25 ng, None | 11868632 | 26% | 56.8% | 17.2% | 59 |
| 13 | 100 ng, 8.75 pmol, 193 pool | 7274252 | 77.0% | 7.1% | 15.9% | 0.13 |
| 14 | 100 ng, 8.75 pmol, 193 pool | 10060012 | 78.2% | 6.7% | 15.0% | 0.17 |
| 15 | 100 ng, 4.38 pmol, 193 pool | 10315535 | 70.8% | 10.4% | 18.9% | 1.3 |
| 16 | 100 ng, 4.38 pmol, 193 pool | 11000478 | 71.2% | 9.9% | 18.9% | 1.8 |
| 17 | 100 ng input, None | 41213818 | 27.1% | 54.5% | 18.4% | 60 |
| 18 | 100 ng input, None | 31358025 | 28.8% | 55.2% | 16.0% | 61 |
| 19 | 500 ng, 8.75 pmol, 193 pool | 11750443 | 77.6% | 6.8% | 15.6% | 0.19 |
| 20 | 500 ng, 8.75 pmol, 193 pool | 21232752 | 77.7% | 6.5% | 15.8% | 0.19 |
| 21 | 500 ng, 8.75 pmol, 96 pool | 10417165 | 60.4% | 25.5% | 14.1% | 19.7 |
| 22 | 500 ng, 4.38 pmol, 96 pool | 13951824 | 51.9% | 33.9% | 14.2% | 25.6 |
| 23 | 500 ng, 4.38 pmol, 193 pool | 11909279 | 72.6% | 9.6% | 17.8% | 1.1 |
| 24 | 500 ng, 4.38 pmol, 193 pool | 9777865 | 74.0% | 8.7% | 17.3% | 1.5 |
| 25 | 500 ng input, None | 20341217 | 27.8% | 56.3% | 15.9% | 53.4 |
| 26 | 500 ng input, None | 11676320 | 28.6% | 55.7% | 15.7% | 53.1 |
| 27 | 1000 ng, 8.75 pmol, 193 pool | 8985310 | 78.5% | 6.3% | 15.2% | 0.19 |
| 28 | 1000 ng, 8.75 pmol, 193 pool | 8228793 | 76.6% | 6.6% | 16.8% | 0.23 |
| 29 | 1000 ng, 8.75 pmol, 96 pool | 11549940 | 61.3% | 25.3% | 13.4% | 15.6 |
| 30 | 1000 ng, 4.38 pmol, 96 pool | 11495247 | 51.5% | 34.7% | 13.8% | 22.5 |
| 31 | 1000 ng, 4.38 pmol, 193 pool | 8281345 | 74.4% | 8.7% | 16.9% | 0.99 |
| 32 | 1000 ng, 4.38 pmol, 193 pool | 8770047 | 74.3% | 8.5% | 17.2% | 1.6 |
| 33 | 1000 ng input, None | 8873922 | 29.6% | 54.9% | 15.5% | 50.6 |
| 34 | 1000 ng input, None | 10202258 | 29.2% | 55.2% | 15.6% | 49.5 |

Summary of the Above Table:

At all RNA input amounts tested, 8.75 pmol each of the pool of 193 blockers worked the best in reducing the amount of read that were rRNA (see Samples 1, 2, 7, 8, 13, 14, 19, 20, 27, and 28). 4.38 pmol each of the 193 pool also worked well but with some reduction in rRNA blocking performance (see Samples 3, 4, 9, 10, 15, 16, 23, 24, 31, and 32).

Sequencing Results for Non-rRNA Genes (Scatter Plots):

Scatter plots were generated to show the gene expression profiles for 11,000 unique non-rRNA genes for input amounts of 25 ng, 100 ng, 500 ng, and 1000 ng using the pool of 193 blockers at 4.38 pmol or 8.75 pmol each blocker. Each dot represents the log 2 of reads for each unique non-rRNA gene normalized to the average of 2 house-keeping genes GAPDH and ACTB. The scatter plots are summarized in Tables A and B below.

### Table A

### Summary of Scatter Plots Comparing Various Types of Replicate Experiments

| Ref. No. | RNA Input (ng) | Blockers (pmol) | R² |
|---|---|---|---|
| 1 | 25 | 8.75 | 0.7828 |
| 2 | 25 | 4.38 | 0.7878 |
| 3 | 25 | none | 0.6965 |
| 4 | 100 | 8.75 | 0.9659 |
| 5 | 100 | 4.38 | 0.9771 |
| 6 | 100 | none | 0.9186 |
| 7 | 500 | 8.75 | 0.9839 |
| 8 | 500 | 4.38 | 0.9829 |
| 9 | 500 | none | 0.8984 |
| 10 | 1000 | 8.75 | 0.9735 |
| 11 | 1000 | 4.38 | 0.9753 |
| 12 | 1000 | none | 0.8691 |

TABLE B
Summary of scatter plots comparing various
types of assays

| | Assay 1 | | Assay 2 | | |
|---|---|---|---|---|---|
| Ref. | RNA Input | Blockers | RNA Input | Blockers | |
| No. | (ng) | (pmol) | (ng) | (pmol) | R² |
| 1 | 25 | None | 25 | 8.75 | 0.8021 |
| 2 | 25 | None | 25 | 4.38 | 0.8157 |
| 3 | 100 | None | 25 | 8.75 | 0.8775 |
| 4 | 100 | None | 25 | 4.38 | 0.8924 |
| 5 | 500 | None | 25 | 8.75 | 0.874 |
| 6 | 500 | None | 25 | 4.38 | 0.883 |
| 7 | 100 | None | 100 | 8.75 | 0.9207 |
| 8 | 100 | None | 100 | 4.38 | 0.939 |
| 9 | 500 | None | 500 | 8.75 | 0.9284 |
| 10 | 500 | None | 500 | 4.38 | 0.9413 |
| 11 | 1000 | None | 1000 | 8.75 | 0.9256 |
| 12 | 1000 | None | 1000 | 4.38 | 0.9328 |
| 13 | 100 | None | 25 | None | 0.8789 |
| 14 | 100 | 8.75 | 25 | 8.75 | 0.9275 |
| 15 | 100 | 4.38 | 25 | 4.38 | 0.9331 |
| 16 | 25 | 4.38 | 25 | 8.75 | 0.8754 |
| 17 | 100 | 4.38 | 100 | 8.75 | 0.9806 |
| 18 | 500 | None | 25 | None | 0.8442 |
| 19 | 500 | 8.75 | 25 | 8.75 | 0.9252 |
| 20 | 500 | 4.38 | 25 | 4.38 | 0.9243 |
| 21 | 500 | 4.38 | 500 | 8.75 | 0.9888 |
| 22 | 1000 | None | 25 | None | 0.8265 |
| 23 | 1000 | 8.75 | 25 | 8.75 | 0.919 |
| 24 | 1000 | 4.38 | 25 | 4.38 | 0.9182 |
| 25 | 1000 | 4.38 | 1000 | 8.75 | 0.9863 |
| 26 | 500 | None | 1000 | None | 0.9397 |
| 27 | 500 | 8.75 | 100 | 8.75 | 0.9836 |
| 28 | 500 | 4.38 | 100 | 4.38 | 0.9828 |
| 29 | 1000 | None | 100 | None | 0.92 |
| 30 | 1000 | 8.75 | 100 | 8.75 | 0.9784 |
| 31 | 1000 | 4.38 | 100 | 4.38 | 0.9768 |
| 32 | 1000 | None | 500 | None | 0.9361 |
| 33 | 1000 | 8.75 | 500 | 8.75 | 0.9892 |
| 34 | 1000 | 4.38 | 500 | 4.38 | 0.9886 |

### Summary of Sequencing Results for Non-rRNA Genes (Scatter Plots)

Because the QIASeq Stranded Total RNA Library Kit has a suggested minimum input of 100 ng total RNA, the results for 25 ng input show that the technical duplicates had poor R² values as expected (see Table A, Ref. Nos. 1 and 2). However, inclusion of the blockers improved R² values as compared to no blockers (compare R² values of Ref. Nos. 1 and 2 with that of Ref. No. 3). This improvement was the result of the blockers enhancing the sensitivity of detection and quantification of non-rRNA genes.

Reproducibility of technical duplicates was good for 100 ng, 500 ng, and 1000 ng input (see Table A, Ref. Nos. 4, 5, 7, 8, 10, and 11), and again was better with blockers compared to no-treatment (compare R² values in Table A between Ref. No. 4 or 5 and Ref. No. 6; between Ref. No. 7 or 8 with Ref. No. 9; and Ref. No. 10 or 11 with Ref. No. 12).

Scatter plots show that there was very good correlation of non-rRNA gene expression profiles between 100 ng, 500 ng, 1000 ng, for all blocker amounts (see Table B, Ref. Nos. 17, 21, 25, 27, 30, 31, 33, and 34, all of which have R² values greater than 0.96), indicating that using the pool of 193 blockers at either 8.75 pmol or 4.38 pmol did not negatively alter gene expression profiles while still effectively eliminating rRNA.

### Example 4

### Designing Blockers for Blocking cDNA Synthesis of Bacterial 5S, 16S and 23S rRNA Sequences

This Example describes the design of blockers for blocking cDNA synthesis of bacterial 5S, 16S and 23S rRNA sequences. This design is applicable for samples that are either single-species (for example E. coli K12) or mixed communities as in complex samples, such as stool, sewage or environmental, where there are potentially thousands of different rRNA sequences.

For design, 5S bacterial rRNA sequences (7,300 total sequences) were downloaded from the 5S rRNA Database (http://combio.pl/rrna/), 16S bacterial rRNA sequences (168,096 total sequences) were downloaded from SILVA (https://www.arb-silva.de/) and 23S bacterial rRNA sequences (592,605 total sequences) were downloaded from SILVA (https://www.arb-silva.de/). As sequences can be continually added, modified or deleted to the databases, future designs could take into account altered numbers of sequences.

The molecular nature of the bacterial rRNA cDNA synthesis blockers are principally similar to those used to block cDNA synthesis of human, mouse and rat rRNA (see blockers B1-6193 described above). The oligonucleotides are (on average) 20 bp in length, spaced (on average) 30 bp apart when tiled antisense against the rRNA sequences, contain LNA oligonucleotides and contain a blocking residue at the 3′ terminus of each of the oligonucleotide. The blockers are expected to block cDNA synthesis of bacterial rRNA in a similar manner to the human, mouse and rat rRNA blockers.

Due to the sheer number of bacterial rRNA sequences, each blocker was picked to increase the total coverage the most when all of the rRNA sequences for a particular rRNA type (whether that is 5S, 16S or 23S) was considered. The blocker is designed to be antisense to the target rRNA sequence of interest. Specifically, after the BLOCKER LENGTH (i.e., about 20 bp), the DISTANCE between neighboring blockers (i.e., about 30 bp) when annealing to a set of target rRNA sequences (e.g., bacterial 5S rRNA), and the NUMBER of blockers to select (e.g., 1000 or 2000) were defined, the following design algorithm was used:

1. Count frequencies of all kmers with K=BLOCKER LENGTH in the set of target sequences,

2. Sort kmers by frequency,

3. Add most frequent kmer to blocker set,

4. Find location of selected kmer in all target sequences,

5. Determine kmers within DISTANCE downstream of kmer location and 0.5 DISTANCE upstream in each target sequence,

6. Decrement kmers identified in step 5 in the frequency list, and

7. Repeat steps 2-6 until the NUMBER of blockers is reached.

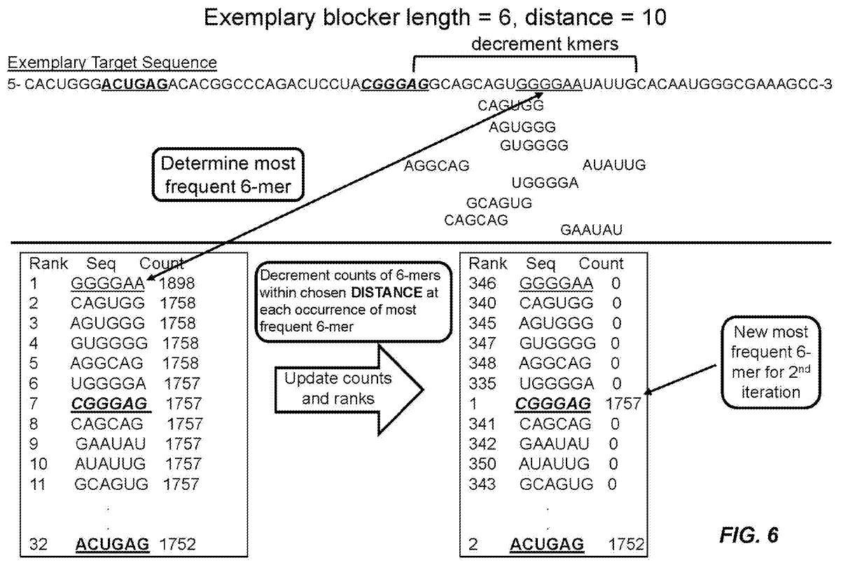

An example of the above process is shown in FIG. 6. In this example, the blocker length is 6 nucleotides, the distance between neighboring blockers is 10. For orientation of the blockers in relation to each other, the blockers are designed antisense to the target rRNA sequence of interest. The first step is to count all possible 6-mers in all target sequences (only one exemplary target sequence shown at the top of FIG. 6), determine the most frequent 6-mer, and rank the 6-mers based on their frequency in the target nucleic acids as shown in the left table. The next step is to decrement counts of 6-mers within the chosen DISTANCE at each occurrence of the most frequent 6-mer, update counts and ranks, and identify the new most frequent 6-mer for the second iteration.

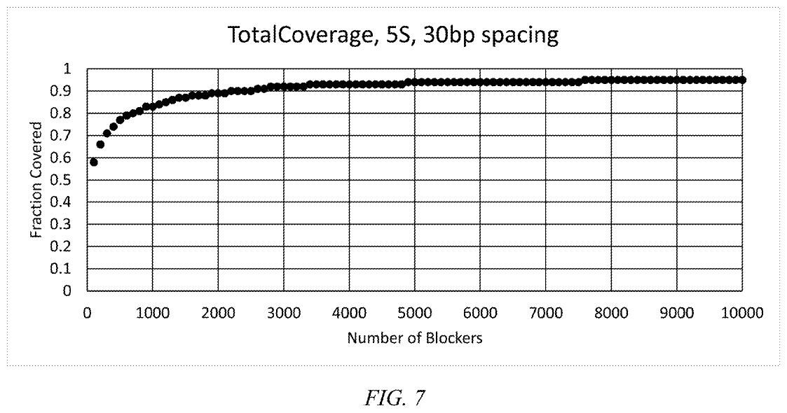

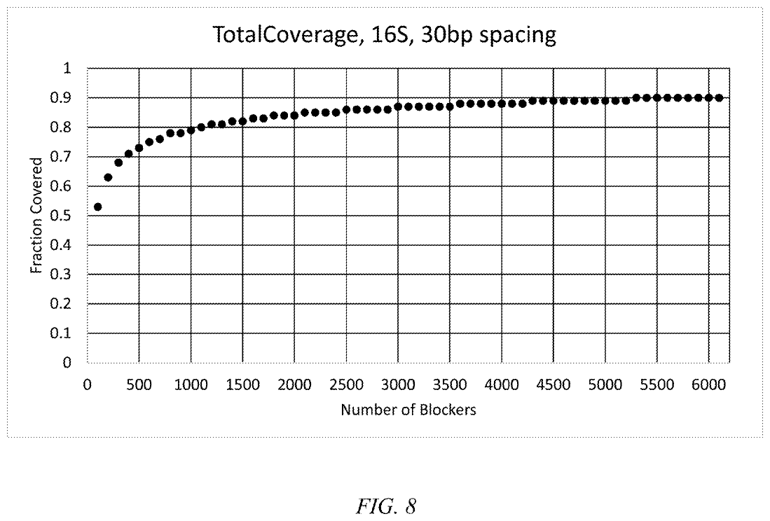

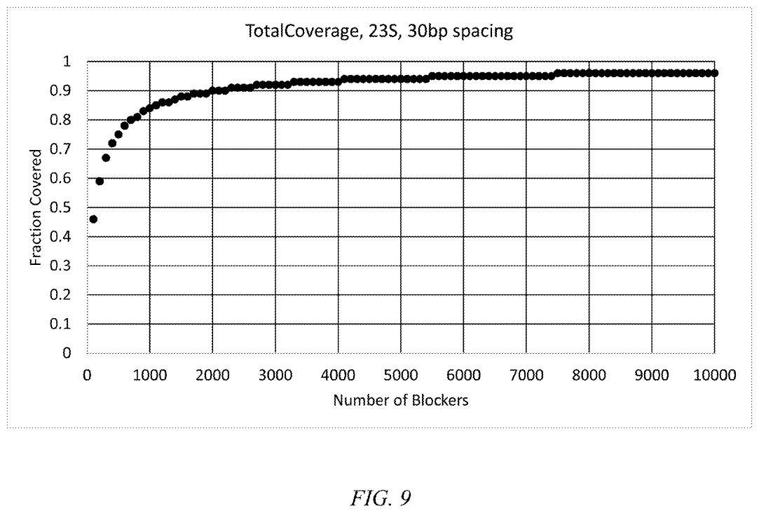

The total fraction of rRNA sequences covered increases when the number of blockers increases (see FIGS. 7-9). For 5S rRNA, 96% of all rRNA sequences is covered with 10,000 blockers when the blockers are 20 bp in length, spaced 30 bp apart (see FIG. 7). For 16S rRNA, 90% of all rRNA sequences is covered with 6,100 blockers when the blockers are 20 bp in length, spaced 30 bp apart (see FIG. 8). For 23S rRNA, 96% of all rRNA sequences is covered with 10,000 blockers when the blockers are 20 bp in length, spaced 30 bp apart (see FIG. 9).

It is not required to include all blockers when attempting to block cDNA synthesis of bacterial rRNA. The coverage was 83% for 5S rRNA (using first 1000 blockers), 84% for 16S rRNA (using first 2000 blockers), and 84% for 23S rRNA (using first 1000 blockers). The sequences of the first 100 blockers for 5S rRNA, 16S rRNA, and 23S rRNA are shown as exemplary blockers in the tables below. 35 nmol of each oligo was synthesized using standard desalt purification. Following synthesis, the four pools were combined together to generate a blocker mix that contained 4000 blockers and was used in Examples 5-8.

The sequences of 100 exemplary blockers for each of bacterial 5S rRNA, 16S RNA and 23S rRNA are provided in the tables below.

| | | |
|---|---|---|
| Name | Oligo Sequence | SEQ ID NO: |

Blockers 5S1-5S100 Sequences

| | | |
|---|---|---|
| 5S1 | + CG + TT + TC + ACTT + CTG + AGT + TC + GG/3AmMO/ | 208 |
| 5S2 | + A0000 + ACA + CTAC + CA + TC + GGC + G/3AmMO/ | 209 |
| 5S3 | + CTTAG + CT + TCCG + GG + TT + CGGAA/3AmMO/ | 210 |
| 5S4 | G + TGT + TC + GGGA + TG + GGA + ACG + GG/3AmMO/ | 211 |
| 5S5 | C + GA + GTT + CG + GG + ATGGG + AT + CGG/3AmMO/ | 212 |
| 5S6 | T + CT + GT + TC + GG + AA + TGGG + AAG + AG/3AmMO/ | 213 |
| 5S7 | A + GC + TTA + AC + TT + CTG + TG + TTC + GG/3AmMO/ | 214 |
| 5S8 | + AG + CTT + AACT + TCCG + TG + TTC + GG/3AmMO/ | 215 |
| 5S9 | T + CCTG + TTC + GG + GATG + GGA + AGG/3AmMO/ | 216 |
| 5S10 | + GGCG + GTGT + CCT + ACT + CT000 + A/3AmMO/ | 217 |
| 5S11 | G + TGT + TCG + GAA + TGG + GAA + CG + GG/3AmMO/ | 218 |
| 5S12 | C + CC + CA + ACT + ACC + ATCG + GCGCT/3AmMO/ | 219 |
| 5S13 | + ATG + AC + CTA + CT + CT + CAC + AT + GG + G/3AmMO/ | 220 |
| 5S14 | + ACT + CTC + GC + ATG + GGGAG + A000/3AmMO/ | 221 |
| 5S15 | + GGCG + GCGT + CCT + ACT + CT000 + A/3AmMO/ | 222 |
| 5S16 | G + TGCA + GTAC + CAT + CGGCG + CTG/3AmMO/ | 223 |
| 5S17 | CC + GAG + TTC + GG + AATG + GG + AT + CG/3AmMO/ | 224 |
| 5S18 | + TG + GCAG + CG + ACCT + ACTCT + CC + C/3AmMO/ | 225 |
| 5S19 | T + GTC + CTA + CTC + TCAC + ATGG + GG/3AmMO/ | 226 |
| 5S20 | G + GCG + GCGAC + CT + ACT + CT000 + A/3AmMO/ | 227 |
| 5S21 | + GA + GTTC + GG + GA + TGGG + AT + CA + GG/3AmMO/ | 228 |
| 5S22 | GT + CCT + AC + TC + TC + ACAGG + GGGA/3AmMO/ | 229 |
| 5S23 | + CTG + CAGT + ACC + ATCGG + CGC + TG/3AmMO/ | 230 |
| 5S24 | + CGG + GTTC + GGG + ATGGG + ACC + GG/3AmMO/ | 231 |
| 5S25 | A + GTAC + CATC + GGCGC + TGG + AGG/3AmMO/ | 232 |
| 5S26 | CT + GTG + TTC + GG + CATG + GG + AA + CA/3AmMO/ | 233 |
| 5S27 | + GC + CTG + GC + AAC + GTCCT + ACTC + T/3AmMO/ | 234 |
| 5S28 | T + GA + CG + AT + GAC + CT + AC + TTT + CA + C/3AmMO/ | 235 |
| 5S29 | + GTGT + TC + GG + GA + TG + GG + AA + CAG + G/3AmMO/ | 236 |
| 5S30 | + TGCCT + GGC + AGTT + CC + CT + ACT + C/3AmMO/ | 237 |
| 5S31 | G + GC + GGT + GA + CCTA + CT + CT000 + A/3AmMO/ | 238 |
| 5S32 | T + GT + TC + GG + AAT + GG + GA + ACA + GG + T/3AmMO/ | 239 |
| 5S33 | CCG + AGTT + CG + AG + ATG + GG + AT + CG/3AmMO/ | 240 |
| 5S34 | GG + CAA + CGAC + CTA + CT + CT000 + A/3AmMO/ | 241 |
| 5S35 | C + AGGG + GGCA + ACC + 000AA + CTA/3AmMO/ | 242 |
| 5S36 | + ACC + ATC + GG + CGC + TGAAG + AGCT/3AmMO/ | 243 |
| 5S37 | A + AT + CCG + CA + CT + ATC + AT + CGG + CG/3AmMO/ | 244 |
| 5S38 | G + GC + GGC + GA + CCTA + CT + CT000 + G/3AmMO/ | 245 |
| 5S39 | T + TCGG + CATG + GGAAC + GGG + TGT/3AmMO/ | 246 |
| 5S40 | G + GG + CT + TA + ACT + TC + TC + TGT + TC + G/3AmMO/ | 247 |
| 5S41 | C + ACAC + CGTC + TCCAG + TGC + AGT/3AmMO/ | 248 |
| 5S42 | + GTT + CGGCG + GTG + TCCT + AC + TTT/3AmMO/ | 249 |
| 5S43 | + CG + GCA + GCGA + CCTA + CT + CT + CC + C/3AmMO/ | 250 |
| 5S44 | + T000 + AAC + TACCA + TC + GG + CGCT/3AmMO/ | 251 |
| 5S45 | + GG + GTTC + GGA + ATGGG + ACCG + GG/3AmMO/MO/ | 252 |
| 5S46 | + ACTC + TCA + CATGG + GG + AG + A000/3Am | 253 |
| 5S47 | A + CGC + AGT + ACC + ATC + GGC + GT + GA/3AmMO/ | 254 |
| 5S48 | + GA + TT + AC + CTAC + TTT + CAC + AC + GG/3AmMO/ | 255 |
| 5S49 | GC + GGC + TACC + TAC + TC + T000A + C/3AmMO/ | 256 |
| 5S50 | T + TC + GG + CAT + GGG + TACA + GGTGT/3AmMO/ | 257 |
| 5S51 | + CTG + AGTT + CGG + CATGG + GGT + CA/3AmMO/ | 258 |
| 5S52 | T + GGC + GAC + GTC + CTAC + TCTC + AC/3AmMO/ | 259 |
| 5S53 | + ACA + CA + GT + CT000 + ATG + CA + GTA/3AmMO/ | 260 |
| 5S54 | C + TG + TGT + TC + GG + TAT + GG + GAA + CA/3AmMO/ | 261 |
| 5S55 | C + GA + TG + AC + CT + AC + TCTC + GCA + TG/3AmMO/ | 262 |
| 5S56 | G + TGCA + GTAC + CAT + CGGCG + CAG/3AmMO/ | 263 |
| 5S57 | GG + CGA + CG + ACCT + ACTC + T000A/3AmMO/ | 264 |
| 5S58 | T + TCG + GC + ATGG + GA + TCA + GGT + GG/3AmMO/ | 265 |
| 5S59 | + TGGC + AGC + GACTT + AC + TC + T000/3AmMO/ | 266 |
| 5S60 | + TC + CTG + TTCG + GAAT + GG + GAA + GG/3AmMO/ | 267 |
| 5S61 | + CCTG + GC + GA + TG + AC + CT + AC + TTT + C/3AmMO/ | 268 |
| 5S62 | + GA + GT + TC + GGAA + TGG + GAT + CA + GG/3AmMO/ | 269 |
| 5S63 | T + GA + GTT + CG + GG + AAG + GG + ATC + AG/3AmMO/ | 270 |
| 5S64 | C + CAC + AC + TA + TCA + TC + GG + CGCT + A/3AmMO/ | 271 |
| 5S65 | + GT + GT + GA + CCTC + TC + TGCCA + TC + A/3AmMO/ | 272 |
| 5S66 | T + TC + GGT + ATG + GG + AA + CGG + GTGT/3AmMO/ | 273 |
| 5S67 | + TCGT + GT + TC + GG + GATG + GG + TACG/3AmMO/ | 274 |
| 5S68 | + CC + CG + GCAAC + GT + CCTAC + TCTC/3AmMO/ | 275 |
| 5S69 | + GCG + CTG + GA + GCG + TTTCA + CGGC/3AmMO/ | 276 |
| 5S70 | + CGC + TGGG + GCG + TTTCA + CGG + CC/3AmMO/ | 277 |
| 5S71 | T + AC + TC + TC + ACA + TG + GG + GAA + AC + C/3AmMO/ | 278 |
| 5S72 | T + T000 + TCAC + GCTAT + GAC + CAC/3AmMO/ | 279 |
| 5S73 | A + TTG + CAG + TAC + CATC + GGCG + CA/3AmMO/ | 280 |
| 5S74 | C + CA + CAC + TAT + CA + TC + GGC + GCTG/3AmMO/ | 281 |
| 5S75 | + AGG + A000 + TGC + GGTCC + AAG + TA/3AmMO/ | 282 |
| 5S76 | A + CCTG + GCGG + CGACC + GAC + TTT/3AmMO/ | 283 |
| 5S77 | G + TGCA + GTAC + CAT + CGCCG + TGC/3AmMO/ | 284 |
| 5S78 | + G0000 + ACAC + TACCA + TC + GGCG/3AmMO/ | 285 |
| 5S79 | C + AC + TTC + TG + AG + TTC + GA + GAT + GG/3AmMO/ | 286 |
| 5S80 | + CCTA + CTC + T000G + CAT + TG + CAT/3AmMO/ | 287 |
| 5S81 | + GT + TC + GA + GATG + GGA + ACA + GG + TG/3AmMO/ | 288 |
| 5S82 | + ACC + ATCGG + CG + CT + AA + AG + AGC + T/3AmMO/ | 289 |
| 5S83 | + GGG + CAGT + ATC + ATCGG + CGC + TG/3AmMO/ | 290 |
| 5S84 | + CTG + GCG + AC + GACCT + ACT + CT + TC/3AmMO/ | 291 |
| 5S85 | TCG + AGTT + CG + GG + ATG + GG + AT + CG/3AmMO/ | 292 |
| 5S86 | GC + CACA + CTA + CC + AT + CGGC + GCT/3AmMO/ | 293 |
| 5S87 | + GC + AGC + TGCG + TTTC + AC + TTC + CG/3AmMO/ | 294 |
| 5S88 | + CATA + GT + AC + CA + TT + AG + CG + CTA + T/3AmMO/ | 295 |
| 5S89 | + AC + CAT + CGG + CG + CA + AAAGA + GC + T/3AmMO/ | 296 |
| 5S90 | C + TG + TG + TT + CG + AC + ATGG + GAA + CA/3AmMO/ | 297 |
| 5S91 | GG + CGA + CG + ACCT + ACTC + T000G/3AmMO/ | 298 |
| 5S92 | + GGCGA + CGTC + CTA + CT + CT000 + A/3AmMO/ | 299 |
| 5S93 | A + ACG + CTA + TGG + TCGC + CAAG + CA/3AmMO/ | 300 |
| 5S94 | TG + CCTG + GCA + GT + GT + CCTA + CTC/3AmMO/ | 301 |
| 5S95 | + GGCGA + CTA + CCT + AC + TC + T000 + A/3AmMO/ | 302 |
| 5S96 | C + GG + CG + CT + AAG + AA + GC + TTA + AC + T/3AmMO/ | 303 |
| 5S97 | G + GG + CT + TA + ACT + GC + TG + TGT + TC + G/3AmMO/ | 304 |
| 5S98 | + GT + GCTA + CTCT + 000AC + A000 + T/3AmMO/ | 305 |
| 5S99 | GG + CAA + CGTC + CTA + CT + CT000 + A/3AmMO/ | 306 |
| 5S100 | G + TCCT + ACTC + TCGCA + GGG + GGA/3AmMO/ | 307 |

Blockers 16S1-16S100 Sequences

| | | |
|---|---|---|
| 16S1 | C + TGCT + GCCT + 000GT + AGG + AGT/3AmMO/ | 308 |
| 16S2 | G + TAT + TAC + CGC + GGCT + GCTG + GC/3AmMO/ | 309 |
| 16S3 | A + CT + AC + CA + GGG + TA + TC + TAA + TC + C/3AmMO/ | 310 |
| 16S4 | + GC + TCG + TT + GC + GGGAC + TTA + ACC/3AmMO/ | 311 |
| 16S5 | + CC + CG + TC + AATT + CCT + TTG + AG + TT/3AmMO/ | 312 |
| 16S6 | T + GAC + GGG + CGG + TGTG + TACA + AG/3AmMO/ | 313 |
| 16S7 | T + GACG + TCAT + 0000A + CCT + TCC/3AmMO/ | 314 |
| 16S8 | + GGTAA + GGT + TCTT + CG + CG + TTG + C/3AmMO/ | 315 |
| 16S9 | C + GAG + CTG + ACG + ACAG + CCAT + GC/3AmMO/ | 316 |
| 16S10 | + TTG + TAGC + AC + GTGT + GT + AG + CC + C/3AmMO/ | 317 |
| 16S11 | C + ACA + TGC + TCC + ACCG + CTTG + TG/3AmMO/ | 318 |
| 16S12 | T + CT + AC + GC + AT + TT + CACC + GCT + AC/3AmMO/ | 319 |
| 16S13 | A + TC + GTT + TA + CG + GCG + TG + GAC + TA/3AmMO/ | 320 |
| 16S14 | + CT + TT + AC + G000 + AGT + AAT + TC + CG/3AmMO/ | 321 |
| 16S15 | + CG + AG + CTG + AC + GA + CAACC + ATG + C/3AmMO/ | 322 |
| 16S16 | C + GCCT + TCGC + CAC + TGGTG + TTC/3AmMO/ | 323 |
| 16S17 | T + TA + CT + AG + CG + AT + TCCG + ACT + TC/3AmMO/ | 324 |
| 16S18 | C + GT + TC + GA + CT + TG + CATG + TGT + TA/3AmMO/ | 325 |
| 16S19 | AC + CTT + GTTAC + GA + CT + TC + A000/3AmMO/ | 326 |
| 16S20 | C + CA + TTG + TG + CAAT + AT + T0000 + A/3AmMO/ | 327 |
| 16S21 | T + TT + AC + AA + CC + CG + AAGG + CCT + TC/3AmMO/ | 328 |
| 16S22 | + CTG + AG + CCA + GG + AT + CAA + AC + TC + T/3AmMO/ | 329 |
| 16S23 | T + CATC + CTCT + CAGAC + CAG + CTA/3AmMO/ | 330 |
| 16S24 | + TT + ACTC + A000 + GT + CCG + CCGC + T/3AmMO/ | 331 |
| 16S25 | T + TACT + CA000 + GT + TC + GCCAC + T/3AmMO/ | 332 |
| 16S26 | + TT + ACTC + A000 + GT + CCG + CCAC + T/3AmMO/ | 333 |
| 16S27 | T + AC + CTC + AC + CA + ACT + AG + CTA + AT/3AmMO/ | 334 |
| 16S28 | + GCCGT + ACTC + 000 + AG + GCGGT + C/3AmMO/ | 335 |
| 16S29 | + CG + CGAT + TA + CT + AGCG + AT + TC + CA/3AmMO/ | 336 |
| 16S30 | + CC + CGGG + AA + CG + TATT + CA + CC + GC/3AmMO/ | 337 |
| 16S31 | C + CA + TTG + TC + CAAT + AT + T0000 + A/3AmMO/ | 338 |
| 16S32 | + CGC + TC + GAC + TT + GC + ATG + TG + TT + A/3AmMO/ | 339 |
| 16S33 | C + TT + TA + CG + CC + CA + ATAA + TTC + CG/3AmMO/ | 340 |
| 16S34 | T + TT + GAG + TT + TT + AAC + CT + TGC + GG/3AmMO/ | 341 |
| 16S35 | T + T000 + AGGTT + GA + GC + CCGGG + G/3AmMO/ | 342 |
| 16S36 | + TA000 + CAC + CAA + CT + AG + CTAA + T/3AmMO/ | 343 |
| 16S37 | T + GAC + GTC + GTC + 000A + CCTT + CC/3AmMO/ | 344 |
| 16S38 | CA + CGCG + GCG + TC + GC + TGCA + TCA/3AmMO/ | 345 |
| 16S39 | + CT + CAG + TC + CCA + GTGTG + GCTG + A/3AmMO/ | 346 |
| 16S40 | + TCA + CC + CTC + TCAG + GTCG + GCT + A/3AmMO/ | 347 |
| 16S41 | + TGC + AG + AC + TCCAA + TCC + GG + ACT/3AmMO/ | 348 |
| 16S42 | C + ACG + CGG + CAT + GGCT + GGAT + CA/3AmMO/ | 349 |
| 16S43 | A + 000 + ACT + 000 + ATGG + TGTG + AC/3AmMO/ | 350 |
| 16S44 | + TACGA + A + T + T + T + CACCT + CT + ACAC/3AmMO/ | 351 |
| 16S45 | + ATC + GT + TTA + GG + GC + GTG + GA + CT + A/3AmMO/ | 352 |
| 16S46 | C + GTAC + T0000 + AG + GC + GGAGT + G/3AmMO/ | 353 |
| 16S47 | + CGC + CTT + CG + CCA + CCGGT + GTTC/3AmMO/ | 354 |
| 16S48 | + GCCGT + ACTC + 000 + AG + GCGGG + G/3AmMO/ | 355 |
| 16S49 | + 000T + CTC + AGGCC + GGC + TA + 000/3AmMO/ | 356 |
| 16S50 | G + TCAG + GC + TTT + CG000 + ATT + GC/3AmMO/ | 357 |
| 16S51 | GG + TAA + GGTTC + TG + CG + CG + TTGC/3AmMO/ | 358 |
| 16S52 | CT + TTCG + CTC + CTCAG + CG + TCAG/3AmMO/ | 359 |
| 16S53 | + CTT + TC + GC + GCCTC + AGC + GT + CAG/3AmMO/ | 360 |
| 16S54 | + T + A + TC + AT + CGA + A + T + T + AA + A + C + C + A + C + A/3AmMO/ | 361 |
| 16S55 | TTT + ACAA + CC + CG + AAG + GC + CG + TC/3AmMO/ | 362 |
| 16S56 | A + TCC + GAACT + GAG + AC + CGGC + TT/3AmMO/ | 363 |
| 16S57 | + TACGC + AT + T + T + CA + CT + GCTA + C + A + C/3AmMO/ | 364 |
| 16S58 | + GG + TAA + GGT + TC + CT + CGCGT + AT + C/3AmMO/ | 365 |
| 16S59 | + CAC + CG + CT + AC + ACC + AG + GAATT + C/3AmMO/ | 366 |
| 16S60 | C + GCCT + TCGC + CAC + CGGTA + TTC/3AmMO/ | 367 |
| 16S61 | A + AG + GGG + CA + TG + ATG + AT + TTG + AC/3AmMO/ | 368 |
| 16S62 | + AT + GCTC + CGCC + GC + TTG + TGCG + G/3AmMO/ | 369 |
| 16S63 | + CT + CAG + TTC + CA + GTGTG + GCTGG/3AmMO/ | 370 |
| 16S64 | T + GCA + TCA + GGC + TTGC + G000 + AT/3AmMO/ | 371 |
| 16S65 | + TA + A + A + T + C + CGGAT + A + AC + GCT + TGC/3AmMO/ | 372 |
| 16S66 | C + CA + AC + AT + CT + CA + CGAC + ACG + AG/3AmMO/ | 373 |
| 16S67 | C + AC + CAA + CA + AG + CTGAT + AG + GCC/3AmMO/ | 374 |
| 16S68 | C + TCAG + T000 + AAT + GTGGC + CGT/3AmMO/ | 375 |
| 16S69 | + CCA + CCGCT + TGT + GCGG + GT + 000/3AmMO/ | 376 |
| 16S70 | T + GCCT + TC + GCCA + TCGG + TGT + TC/3AmMO/ | 377 |
| 16S71 | A + TC + GT + TT + AC + AG + CGTG + GAC + TA/3AmMO/ | 378 |
| 16S72 | T + CACT + CACGC + GG + CG + TTGCT + C/3AmMO/ | 379 |
| 16S73 | TT + CGC + G + TTGC + A + T + CG + AA + TTAA/3AmMO/ | 380 |
| 16S74 | CT + CAGTC + CCA + GTGT + GG + CCGG/3AmMO/ | 381 |
| 16S75 | + AA + GGGC + CA + TG + AGGA + CT + TG + AC/3AmMO/ | 382 |
| 16S76 | + GCT + TTC + GC + ACCTC + AGC + GT + CA/3AmMO/ | 383 |
| 16S77 | T + CG + ACT + TG + CA + TGT + AT + TAG + GC/3AmMO/ | 384 |
| 16S78 | TA + AGGG + GCA + TGAT + G + A + CTT + G + A/3AmMO/ | 385 |
| 16S79 | C + TG + AG + CC + ATG + AT + CA + AAC + TC + T/3AmMO/ | 386 |
| 16S80 | GG + GGTC + GAG + TTGCA + GA + 0000/3AmMO/ | 387 |
| 16S81 | T + TG + TCC + AA + AA + TTC + CC + CAC + TG/3AmMO/ | 388 |
| 16S82 | C + TG + CG + AT + TA + CT + AGCG + ACT + CC/3AmMO/ | 389 |
| 16S83 | G + CAC + CAAT + CC + AT + CTC + TG + GA + A/3AmMO/ | 390 |
| 16S84 | C + GCT + 000 + TTT + ACAC + CCAG + TA/3AmMO/ | 391 |
| 16S85 | T + AA + GG + AC + AA + GG + GTTG + CGC + TC/3AmMO/ | 392 |
| 16S86 | TG + CAGAC + TGC + GATC + CG + GACT/3AmMO/ | 393 |
| 16S87 | T + TA + CT + AG + CG + AT + TCCA + GCT + TC/3AmMO/ | 394 |
| 16S88 | A + AAG + GATA + AG + GG + TTG + CG + CT + C/3AmMO/ | 395 |
| 16S89 | T + TG + TAG + TAC + GT + GT + GTA + G000/3AmMO/ | 396 |
| 16S90 | A + CC + GG + CAG + TCT + CCTT + AGAGT/3AmMO/ | 397 |
| 16S91 | + GGCA + GTC + TCCTT + TG + AG + TTCC/3AmMO/ | 398 |
| 16S92 | + ACCG + TACT + 000 + CAG + GCGGT + C/3AmMO/ | 399 |
| 16S93 | + GC + TTTCG + TGCA + TG + AG + CGT + CA/3AmMO/ | 400 |
| 16S94 | C + TT + TC + GA + GCCTC + AG + CG + TCA + G/3AmMO/ | 401 |
| 16S95 | + GCTT + TC + GC + AC + CTGA + GC + GTCA/3AmMO/ | 402 |
| 16S96 | + CTCAG + T000 + AGTGT + GG + CCGA/3AmMO/ | 403 |
| 16S97 | + CCG + TACT + 000 + CAGGC + GGA + AT/3AmMO/ | 404 |
| 16S98 | + TTTA + CAAT + C + CGAAG + A + C + CTT + C/3AmMO/ | 405 |
| 16S99 | + GCTC + 0000T + C + G + CGGG + TTGG + C/3AmMO/ | 406 |
| 16S100 | + GGG + CT + TTC + AC + AT + CAG + AC + TT + A/3AmMO/ | 407 |

Blockers 23S1-23S100 Sequences

| | | |
|---|---|---|
| 23S1 | A + AG + GA + AT + TT + CG + CTAC + CTT + AG/3AmMO/ | 408 |
| 23S2 | C + CG + AC + AT + CGA + GG + TG + CCA + AA + C/3AmMO/ | 409 |
| 23S3 | + GG + TCG + GAA + CT + TA000 + GACAA/3AmMO/ | 410 |
| 23S4 | + GAA + CTG + TC + TCACG + ACG + TT + CT/3AmMO/ | 411 |
| 23S5 | C + TT + TTA + TC + CG + TTGAG + CG + ATG/3AmMO/ | 412 |
| 23S6 | + CTTT + CC + CT + CA + CGGT + AC + TGGT/3AmMO/ | 413 |
| 23S7 | AC + CTT + CC + AGCA + CCGG + GCAGG/3AmMO/ | 414 |
| 23S8 | + GG + CT + GCT + TC + TAAGC + CA + ACA + T/3AmMO/ | 415 |
| 23S9 | + GGCG + AAC + AG000 + AA + CC + CTTG/3AmMO/ | 416 |
| 23S10 | G + TG + AG + CT + AT + TA + CGCA + CTC + TT/3AmMO/ | 417 |
| 23S11 | T + TAC + GGC + CGC + CGTT + TACT + GG/3AmMO/ | 418 |
| 23S12 | GG + TCCT + CTC + GT + AC + TAGG + AGC/3AmMO/ | 419 |
| 23S13 | T + TAC + GCCAT + TCG + TG + CAGG + TC/3AmMO/ | 420 |
| 23S14 | + TT + TC + GG + GGA + GAACC + AG + CTA + T/3AmMO/ | 421 |
| 23S15 | + CC + CT + TCT + CC + CGAAG + TT + ACG + G/3AmMO/ | 422 |
| 23S16 | G + GCG + ACCGC + CC + CAG + TCAAA + C/3AmMO/ | 423 |
| 23S17 | + T + T + T + A + A + ATGG + C + G + A + A + C + AGCC + A + T/3AmMO/ | 424 |
| 23S18 | G + TG + AG + CT + ATT + AC + GC + TTT + CT + T/3AmMO/ | 425 |
| 23S19 | + GA + C + C + C + A + T + T + A + TA + CAA + A + AGGTA/3AmMO/ | 426 |
| 23S20 | + GGTAC + T + TA + G + ATG + TTT + CAG + TT/3AmMO/ | 427 |
| 23S21 | + CCTG + TGT + CGGTT + TG + CG + GTAC/3AmMO/ | 428 |
| 23S22 | + GAG + ACCG + 000 + CAGTC + AAA + CT/3AmMO/ | 429 |
| 23S23 | + CCT + CC + CAC + CTAT + CCTA + CAC + A/3AmMO/ | 430 |
| 23S24 | + AG + TAA + AGGT + TCAC + GG + GGT + CT/3AmMO/ | 431 |
| 23S25 | + GT + AT + TT + AGCC + TTG + GAG + GA + TG/3AmMO/ | 432 |
| 23S26 | C + 000G + TTAC + ATC + TTCCG + CGC/3AmMO/ | 433 |
| 23S27 | G + GTAT + CAGC + CTG + TTATC + 000/3AmMO/ | 434 |
| 23S28 | + CC + CA + GG + ATGT + GA + TGAGC + CG + A/3AmMO/ | 435 |
| 23S29 | T + TT + CAG + GT + TC + TAT + TT + CAC + TC/3AmMO/ | 436 |
| 23S30 | G + GGAC + CTTA + GCT + GGCGG + TCT/3AmMO/ | 437 |
| 23S31 | T + AG + ATG + CT + TT + CAG + CA + CTT + AT/3AmMO/ | 438 |
| 23S32 | TC + TCG + CAGT + CAA + GC + T000T + T/3AmMO/ | 439 |
| 23S33 | T + TT + CGG + AG + AG + AAC + CA + GCT + AT/3AmMO/ | 440 |
| 23S34 | G + CT + AG + CC + CTA + AA + GC + TAT + TT + C/3AmMO/ | 441 |
| 23S35 | C + AG + CA + TT + CGC + AC + TT + CTG + AT + A/3AmMO/ | 442 |
| 23S36 | AC + GGC + AG + AT + AG + GGACC + GAAC/3AmMO/ | 443 |
| 23S37 | + TTA + CGGC + CGC + CGTTT + ACC + GG/3AmMO/ | 444 |
| 23S38 | + GCA + CCGG + GCA + GGCGT + CAC + AC/3AmMO/ | 445 |
| 23S39 | C + CGA + GTT + CTC + TCAA + GCGC + CT/3AmMO/ | 446 |
| 23S40 | G + CG + CTA + CC + TA + AAT + AG + CTT + TC/3AmMO/ | 447 |
| 23S41 | A + CCTG + TG + TCG + GTTTG + GGG + TA/3AmMO/ | 448 |
| 23S42 | + CT + CG + GT + TGAT + TTC + TTT + TC + CT/3AmMO/ | 449 |
| 23S43 | C + ATT + TTGC + CT + AG + TTC + CT + TC + A/3AmMO/ | 450 |
| 23S44 | + TT + AGC + A000G + CCGT + GT + GTC + T/3AmMO/ | 451 |
| 23S45 | G + GGGT + CTTT + CCGTC + CTG + TCG/3AmMO/ | 452 |
| 23S46 | + GG + AG + AT + AAGC + CT + GTTAT + CC + C/3AmMO/ | 453 |
| 23S47 | TT + ACG + CCTTT + CG + TG + CG + GGTC/3AmMO/ | 454 |
| 23S48 | + CTGT + G + T + T + T + TT + AA + TA + AAC + A + G + T/3AmMO/ | 455 |
| 23S49 | + TCG + ACTA + CGC + CTTTC + GGC + CT/3AmMO/ | 456 |
| 23S50 | G + CC + CTA + TT + CA + GACTC + GC + TTT/3AmMO/ | 457 |
| 23S51 | G + GT + TT + CC + CC + AT + TCGG + AAA + TC/3AmMO/ | 458 |
| 23S52 | + TC + AT + T + C + T + A + CA + AAA + GGC + A + C + G + C/3AmMO/ | 459 |
| 23S53 | A + CA + CT + GC + AT + CT + TCAC + AGC + GA/3AmMO/ | 460 |
| 23S54 | T + GAG + TCT + CGG + GTGG + AGAC + AG/3AmMO/ | 461 |
| 23S55 | C + TC + CGT + TA + CT + CTT + TA + GGA + GG/3AmMO/ | 462 |
| 23S56 | C + AG + AAC + CAC + CG + GA + TCA + CTAT/3AmMO/ | 463 |
| 23S57 | + CTT + CC + CA + CATCG + TTT + CC + CAC/3AmMO/ | 464 |
| 23S58 | C + GAA + ACA + GTG + CTCT + A000 + CC/3AmMO/ | 465 |
| 23S59 | + AGC + 000G + GTA + CATTT + TCG + GC/3AmMO/ | 466 |
| 23S60 | C + CA + CAT + CCT + TT + TC + CAC + TTAA/3AmMO/ | 467 |
| 23S61 | + CTG + T + G + T + T + T + T + T + GA + TAA + ACA + GT/3AmMO/ | 468 |
| 23S62 | C + GA + GT + TC + CTT + AA + CG + AGA + GT + T/3AmMO/ | 469 |
| 23S63 | + CTG + GGCT + GTT + T000T + TTC + GA/3AmMO/ | 470 |
| 23S64 | CA + T000G + GTC + CTCT + CG + TACT/3AmMO/ | 471 |
| 23S65 | T + GG + GAA + AT + CT + CAT + CT + TGA + GG/3AmMO/ | 472 |
| 23S66 | + GTAC + AG + GA + AT + AT + CA + AC + CTG + T/3AmMO/ | 473 |
| 23S67 | + GG + AACC + AC + CG + GATC + AC + TA + AG/3AmMO/ | 474 |
| 23S68 | + TT + ACAG + AA + CG + CTCC + CC + TA + CC/3AmMO/ | 475 |
| 23S69 | G + TC + TC + TCG + TTG + AGAC + AGTGC/3AmMO/ | 476 |
| 23S70 | TG + CTT + CT + AAGC + CAAC + CTCCT/3AmMO/ | 477 |
| 23S71 | A + TC + AA + TT + AAC + CT + TC + CGG + CA + C/3AmMO/ | 478 |
| 23S72 | C + CAT + TCTG + AG + GG + AAC + CT + TT + G/3AmMO/ | 479 |
| 23S73 | A + GGCA + TCCA + CCG + TGCGC + CCT/3AmMO/ | 480 |
| 23S74 | + TTG + GA + ATT + TC + TC + CGC + TA + CC + C/3AmMO/ | 481 |
| 23S75 | C + CGT + TTC + GCT + CGCC + GCTA + CT/3AmMO/ | 482 |
| 23S76 | A + GA + TG + CT + TTC + AG + CG + GTT + AT + C/3AmMO/ | 483 |
| 23S77 | + GT + TA + CC + CAAC + CT + TCAAC + CT + G/3AmMO/ | 484 |
| 23S78 | + CG + GTC + CT + CC + AGTTA + GTG + TTA/3AmMO/ | 485 |
| 23S79 | + CC + CG + TTCGC + TC + GCCGC + TACT/3AmMO/ | 486 |
| 23S80 | C + CGG + GGT + TCT + TTTC + GCCT + TT/3AmMO/ | 487 |
| 23S81 | TT + CAT + CG + CCT + CTG + ACTG + CC + A/3AmMO/ | 488 |
| 23S82 | G + AA + CC + CTT + GGT + CTTC + CGGCG/3AmMO/ | 489 |
| 23S83 | C + AA + ACA + GT + GC + TCT + AC + CTC + CA/3AmMO/ | 490 |
| 23S84 | + CG + ATTA + ACGT + TG + G + A + C + A + G + G + A + A/3AmMO/ | 491 |
| 23S85 | T + TTT + CAACA + T + T + AGTCG + G + T + T + C/3AmMO/ | 492 |
| 23S86 | + CTTA + GA + GG + CT + TT + TC + CT + GGA + A/3AmMO/ | 493 |
| 23S87 | T + TG + GT + AAG + TCG + GGAT + GA000/3AmMO/ | 494 |
| 23S88 | + GG + ACCT + TAG + CTGGT + GGTC + TG/3AmMO/ | 495 |
| 23S89 | + G + TAC + AGGAA + TATT + A + A + C + CT + GT/3AmMO/ | 496 |
| 23S90 | + CC + CA + GGATG + CG + ACGAG + CCGA/3AmMO/ | 497 |
| 23S91 | C + TGC + TTGT + AC + GT + ACA + CG + GT + T/3AmMO/ | 498 |
| 23S92 | + CC + CAG + GATGC + GATG + AG + CCG + A/3AmMO/ | 499 |
| 23S93 | + AT + CA + CCG + GG + TTTCG + GG + TCT + A/3AmMO/ | 500 |
| 23S94 | + GCCT + TTCA + 000 + CCA + GCCAC + A/3AmMO/ | 501 |
| 23S95 | + TT + ATCG + T + TAC + TTA + T + G + T + CAG + C/3AmMO/ | 502 |
| 23S96 | + TCGA + CTC + A000T + GCC + CC + GAT/3AmMO/ | 503 |
| 23S97 | G + CT + TAT + GC + CA + TTG + CA + CTA + AC/3AmMO/ | 504 |
| 23S98 | + GC + TCCTA + CCTA + TC + CT + GTA + CA/3AmMO/ | 505 |
| 23S99 | A + TC + GTA + AC + TC + GCC + GG + TTC + AT/3AmMO/ | 506 |
| 23S100 | T + TAAA + G + G + G + TGGT + AT + T + T + CA + AG/3AmMO/ | 507 |

### Example 5

### Blocking Bacterial rRNAs with Blockers

This Example describes blocking bacterial rRNAs with the blocker mix as described in Example 4. The amount related to a blocker mix described in this Example is the amount of each blocker in the blocker mix. For example, 2.9 pmol blocker mix refers to a block mix contains 2.9 pmol of each blocker.

### Experimental Details

i. RNA (100 ng of Turbo DNase treated total RNA):
1. E. coli Total RNA (ThermoFisher Scientific, Catalog No. AM7940, “E. coli sample”)
2. Gut Microbiome Whole Cell Mix (ATCC, Catalog No. MSA-2006, “ATCC gut sample”)

ii. Blocker depletion procedure
1. Combine the blocker mix (No Blockers, 2.9 pmol, 1.45 pmol and 0.73 pmol) with total RNA (100 ng) and 1×FH Buffer (50 mM Tris pH 8.0, 40 mM KCl, 3 mM MgCl₂) in a final reaction volume of 15 μl (H₂O was used to bring the final reaction to 15 μl)
2. Reaction was heated for 8 min at 89° C., followed by 2 min at 75° C., 2 min at 70° C., 2 min at 65° C., 2 min at 60° C., 2 min at 55° C., 2 min at 37° C., and 2 min at 25° C.
3. 1.3× (beads to sample v/v ratio) bead cleanup was performed (this was not performed in experimental conditions noted as “No Cleanup”):
   a. Add 19.5 μl QIAseq Beads (pre-warmed to room temperature) to the 15 μl reaction. Mix thoroughly by vortexing, and incubate for 5 min at room temperature.
   b. Centrifuge in a table top centrifuge until the beads are completely pelted (˜2 min).
   c. Place the tubes/plate on a magnetic rack for 2 min. Once the solution has cleared, with the beads still on the magnetic stand, carefully remove and discard the supernatant.
   d. With the beads still on the magnetic stand, add 200 μl of 80% ethanol. Rotate the tube (2 to 3 times) or move the plate side-to-side between the two column positions of the magnet to wash the beads. Carefully remove and discard the wash.
   e. Repeat the ethanol wash, and completely remove all traces of the ethanol wash after this second wash.
   f. With the beads still on the magnetic stand, air dry at room temperature for 10 min.
   g. Remove the beads from the magnetic stand, and elute the nucleic acid from the beads by adding 31 μl nuclease-free water. Mix well by pipetting.
   h. Return the tube/plate to the magnetic rack until the solution has cleared.
   i. Transfer 29 μl of the supernatant to clean tubes/plate.
iii. QIAseq Stranded RNA library preparation
   1. Set up and perform first-strand synthesis reaction associated with the QIAseq Stranded Total RNA Library Kit:

| Component | Volume/reaction |
|---|---|
| RNA from bead cleanup | 29 μl |
| reaction | |
| Diluted DTT (0.4M) | 1 μl |
| RT Enzyme | 1 μl |
| 5x RT Buffer | 8 μl |
| RNase Inhibitor | 1 μl |
| Total volume | 40 μl |

2. Prepare remaining QIAseq Stranded library according to the user manual
iv. Perform next-generation sequencing
   1. Use Illumina NextSeq 500 system with 150 cycles (75×2 paired end)
v. Perform data analysis using CLC Genomics Workbench.

### Results

The results are shown in the table below.

| RNA | Amount | | OD | % NGS | | % NGS | # Genes | # Genes |
|---|---|---|---|---|---|---|---|---|
| (Turbo | of each | | (ng/ul) | Reads | % NGS | Reads | Detected | Detected |
| DNase | blocker | | of NGS | Mapped | Reads | Mapped | (FPKM | (FPKM |
| treated) | (pmol) | Cleanup | Library | in Pairs | Unmapped | to rRNA | >0.3) | >3.0) |
| 100 ng | No | No | 12 | 85.47 | 13.29 | 97.79 | 3302 | 3235 |
| E.coli | blockers | Cleanup | | | | | | |
| 100 ng | No | 1.3x QIAseq | 10 | 87.85 | 10.7 | 97.08 | 3549 | 3364 |
| E.coli | blockers | Beads | | | | | | |
| 100 ng | 2.90 | 1.3x QIAseq | 4 | 94.96 | 3.71 | 3.59 | 4196 | 3408 |
| E.coli | | Beads | | | | | | |
| 100 ng | 1.45 | 1.3x QIAseq | 8 | 94.44 | 3.78 | 2.73 | 4222 | 3410 |
| E.coli | | Beads | | | | | | |
| 100 ng | 0.73 | 1.3x QIAseq | 8 | 94.42 | 3.82 | 4.08 | 4237 | 3431 |
| E.coli | | Beads | | | | | | |
| 100 ng | No | No Cleanup | 11 | 86.18 | 12.44 | 96.35 | 19737 | 16678 |
| ATCC Gut | blockers | | | | | | | |
| 100 ng | No | 1.3x QIAseq | 10 | 86.50 | 12.04 | 95.32 | 23601 | 18349 |
| ATCC Gut | blockers | Beads | | | | | | |
| 100 ng | 2.90 | 1.3x QIAseq | 4 | 89.96 | 8.41 | 12.32 | 28471 | 17373 |
| ATCC Gut | | Beads | | | | | | |
| 100 ng | 1.45 | 1.3x QIAseq | 12 | 90.82 | 7.45 | 23.44 | 29279 | 17755 |
| ATCC Gut | | Beads | | | | | | |
| 100 ng | 0.73 | 1.3x QIAseq | 14 | 90.74 | 7.45 | 34.29 | 29296 | 17768 |
| ATCC Gut | | Beads | | | | | | |

### FPKM: Fragments Per Kilobase of Exon Per Million Reads

The results show:

No blockers for both samples (E. coli and ATCC gut) resulted in a high percentage of rRNA.

2.9 pmol blockers gave the best performance with respect to rRNA blocking with both E. coli and ATCC gut samples.

For the E. coli sample, decreasing the amount of blockers had negligible effect on rRNA blocking. However, for the ATCC gut sample, when the amount of blocker was reduced, the amount of reads mapped to rRNA increased.

rRNA blocking led to an increased number of genes detected.

The blocking efficacy is inconsistent with that predicted by the blocker design algorithm: For the E. coli sample, the design algorithms predicted the blocking efficacy to be 93% of 5S, 99% of 16S, and 99% of 23S. The above results shown that in practice, this was achieved as 97% of all rRNA was removed.

### Conclusion

Bacterial rRNA blockers reduced reads mapped to rRNA from about 97% to about 3% for the E. coli sample and from about 95% to about 12% for the ATCC gut sample.

### Example 6

### Blocking Bacterial rRNAs with Blockers at Different Amounts and with Different Bead Cleanup Steps

This Example describes blocking bacterial rRNAs with the blocker mix as described in Example 4 at different concentrations and with different bead cleanup steps. Similar to Example 5, the amount related to a blocker mix described in this Example is the amount of each blocker in the blocker mix.

In this Example, the ATCC gut sample as described in Example 5 was used as the RNA sample. The method and materials were the same as in Example 5 except that the amounts of the block mix used in this Example were 2.9 pmol and 5.8 pmol, and that two versions of bead cleanups were performed: one (“one round”) was the same as in Example 5, the other (“two rounds”) had the following additional steps between steps 3.c. and 3.d.:

(i) Add 15 μl of nuclease-free water and 19.5 μl of QIAseq NGS Bead Binding Buffer. Mix thoroughly by vortexing, and incubate for 5 min at room temperature.

(ii) Centrifuge in a table top centrifuge until the beads are completely pelted (about 2 min).

(iii) Place the tubes/plate on a magnetic rack for 2 min. Once the solution has cleared, with the beads still on the magnetic stand, carefully remove and discard the supernatant.

### Results

The results are shown in the table below.

| RNA | Amount | | OD | % NGS | | % NGS | # Genes | # Genes |
|---|---|---|---|---|---|---|---|---|
| (Turbo | of each | | (ng/ul) | Reads | % NGS | Reads | Detected | Detected |
| DNase | blocker | | of NGS | Mapped | Reads | Mapped | (FPKM | (FPKM |
| treated) | (pmol) | Cleanup | Library | in Pairs | Unmapped | to rRNA | >0.3) | >3.0) |
| 100 ng | 2.9 | 1 round | 10 | 90.49 | 8.09 | 15.13 | 29153 | 17592 |
| ATCC | | 1.3x | | | | | | |
| Gut | | QIAseq | | | | | | |
| | | Beads | | | | | | |
| 100 ng | 5.8 | 1 round | 3 | 87.54 | 11.02 | 4.84 | 25042 | 15767 |
| ATCC | | 1.3x | | | | | | |
| Gut | | QIAseq | | | | | | |
| | | Beads | | | | | | |
| 100 ng | 2.9 | 2 rounds | 10 | 89.18 | 8.85 | 19.61 | 29224 | 17800 |
| ATCC | | 1.3x | | | | | | |
| Gut | | QIAseq | | | | | | |
| | | Beads | | | | | | |
| 100 ng | 5.8 | 2 rounds | 3 | 87.83 | 10.46 | 6.83 | 27042 | 16568 |
| ATCC | | 1.3x | | | | | | |
| Gut | | QIAseq | | | | | | |
| | | Beads | | | | | | |

The results show: Doubling the amount of blocker from 2.9 pmol to 5.8 pmol improved depletion of rRNA.

NGS libraries prepared when 5.8 pmol blocker mix was used had a low concentration.

Even though the use of 5.8 pmol blocker mix resulted in improved rRNA depletion, it resulted in fewer genes positively called, whether the cutoff was an FPKM of 0.3 or 3.0.

2 rounds of 1.3× bead cleanup had a neutral effect.

### Conclusion

While 5.8 pmol blocker mix was more effective in rRNA depletion, 2.9 pmol may be more preferred when both rRNA depletion and positively expressed genes are considered.

### Example 7

### Blocking Bacterial rRNAs with Blockers at Different Amounts and with Different Bead Cleanup Steps

This Example also describes blocking bacterial rRNAs with the blocker mix as described in Example 4 at different concentrations and with different bead cleanup steps. Similar to Example 5, the amount related to a blocker mix described in this Example is the amount of each blocker in the blocker mix.

In this Example, the ATCC gut sample as described in Example 5 was used as the RNA sample. The method and materials were the same as in Example 6 except that the amounts of the block mix used in this Example were 2.9 pmol, 4.35 pmol, and 5.8 pmol.

### Results

The results are shown in the table below.

| | | | | % | | % | |
|---|---|---|---|---|---|---|---|
| | | | | NGS | % | NGS | # |
| RNA | Amount | | OD | Reads | NGS | Reads | Genes |
| (Turbo | of each | | (ng/ul) | Mapped | Reads | Mapped | Detected |
| DNase | blocker | | of NGS | in | Un- | to | (FPKM |
| treated) | (pmol) | Cleanup | Library | Pairs | mapped | rRNA | >0) |
| 100 ng | No | No | 13 | 86.83 | 11.51 | 96.51 | 20696 |
| ATCC | blockers | cleanup | | | | | |
| Gut | | | | | | | |
| 100 ng | No | 1 round | 11 | 86.2 | 12.18 | 95.51 | 23093 |
| ATCC | blockers | 1.3x | | | | | |
| Gut | | QIAseq | | | | | |
| | | Beads | | | | | |
| 100 ng | No | 2 round | 10 | 85.65 | 12.71 | 95.39 | 23846 |
| ATCC | blockers | 1.3x | | | | | |
| Gut | | QIAseq | | | | | |
| | | Beads | | | | | |
| 100 ng | 2.9 | 1 round | 4 | 89.63 | 9.07 | 10.06 | 28932 |
| ATCC | | 1.3x | | | | | |
| Gut | | QIAseq | | | | | |
| | | Beads | | | | | |
| 100 ng | 2.9 | 2 round | 5 | 90.03 | 8.58 | 14.67 | 31569 |
| ATCC | | 1.3x | | | | | |
| Gut | | QIAseq | | | | | |
| | | Beads | | | | | |
| 100 ng | 4.35 | 1 round | 3 | 87.02 | 11.55 | 6.78 | 25091 |
| ATCC | | 1.3x | | | | | |
| Gut | | QIAseq | | | | | |
| | | Beads | | | | | |
| 100 ng | 4.35 | 2 round | 4 | 89.42 | 9.05 | 11.3 | 29802 |
| ATCC | | 1.3x | | | | | |
| Gut | | QIAseq | | | | | |
| | | Beads | | | | | |
| 100 ng | 5.8 | 1 round | 3 | | | | |
| ATCC | | 1.3x | | | | | |
| Gut | | QIAseq | | | | | |
| | | Beads | | | | | |
| 100 ng | 5.8 | 2 round | 3 | 88 | 10.53 | 7.86 | 25029 |
| ATCC | | 1.3x | | | | | |
| Gut | | QIAseq | | | | | |
| | | Beads | | | | | |

The results show:

Increasing blockers from 2.9 pmol to 4.35 pmol and further to 5.8 pmol improved depletion of rRNA.

NGS libraries prepared using 5.8 pmol blocker mix had a low concentration, regardless of the number of rounds of bead cleanups.

2 rounds of 1.3× bead cleanup improved the number of genes detected, but also increased rRNA percentage. On balance, it is more desirable to have an increased number of genes detected.

Reads mapped in pairs also increase with 2 rounds of 1.3× bead cleanup.

### Conclusion

The combination of 2.9 pmol of blocker mix and 2 rounds of 1.3× bead cleanup provides the most desirable results.

### Example 8

### Blocking Bacterial rRNAs with Blockers with Different Bead Cleanup Steps

This Example also describes blocking bacterial rRNAs with the blocker mix as described in Example 4 with different bead cleanup steps. Similar to Example 5, the amount related to a blocker mix described in this Example is the amount of each blocker in the blocker mix.

In this Example, two different RNA samples were used. One was the ATCC gut sample as described in Example 5 was used as the RNA sample. The other (“ATCC 3 Mix) was the mixture of the following:

a. 20 Strain Even Mix Whole Cell Material (ATCC, cat. no. MSA-2002)

b. Skin Microbiome Whole Cell Mix (ATCC, cat. no. MSA-2005)

c. Oral Microbiome Whole Cell Mix (ATCC, cat. no. MSA-2004)

The method and materials were otherwise the same as in Example 6 except that the amount of the block mix used in this Example was 2.9 pmol.

### Results

The results are shown in the table below.

| | | | | | | | # |
|---|---|---|---|---|---|---|---|
| RNA | Amount | | OD | % NGS | | % NGS | Genes |
| (Turbo | of each | | (ng/ul) | Reads | % NGS | Reads | Detected |
| DNase | blocker | | of NGS | Mapped | Reads | Mapped | (FPKM |
| treated) | (pmol) | Cleanup | Library | in Pairs | Unmapped | to rRNA | >0) |
| 100 ng | No | 1 round | 5 | 85.69 | 12.85 | 95.48 | 21778 |
| ATCC | blockers | 1.3x | | | | | |
| Gut | | QIAseq | | | | | |
| | | Beads | | | | | |
| 100 ng | No | 1 round | 11 | 86.31 | 12.19 | 95.4 | 22225 |
| ATCC | blockers | 1.3x | | | | | |
| Gut | | QIAseq | | | | | |
| | | Beads | | | | | |
| 100 ng | 2.9 | 1 round | 5 | 89.36 | 9 | 13.38 | 27748 |
| ATCC | | 1.3x | | | | | |
| Gut | | QIAseq | | | | | |
| | | Beads | | | | | |
| 100 ng | 2.9 | 1 round | 3 | 89.59 | 9.04 | 13.75 | 24697 |
| ATCC | | 1.3x | | | | | |
| Gut | | QIAseq | | | | | |
| | | Beads | | | | | |
| 100 ng | No | 2 round | 7 | 85.92 | 12.54 | 95.48 | 21906 |
| ATCC | blockers | 1.3x | | | | | |
| Gut | | QIAseq | | | | | |
| | | Beads | | | | | |
| 100 ng | No | 2 round | 7 | 86.38 | 12.13 | 95.45 | 22283 |
| ATCC | blockers | QIAseq | | | | | |
| Gut | | 1.3x | | | | | |
| | | Beads | | | | | |
| 100 ng | 2.9 | 2 round | 6 | 90.21 | 8.24 | 20.94 | 28915 |
| ATCC | | 1.3x | | | | | |
| Gut | | QIAseq | | | | | |
| | | Beads | | | | | |
| 100 ng | 2.9 | 2 round | 5 | 90.03 | 8.37 | 19.19 | 28386 |
| ATCC | | 1.3x | | | | | |
| Gut | | QIAseq | | | | | |
| | | Beads | | | | | |
| 100 ng | No | 1 round | 8 | 76.36 | 21.77 | 94.52 | 28486 |
| ATCC 3 | blockers | 1.3x | | | | | |
| Mix (20 | | QIAseq | | | | | |
| Strain + | | Beads | | | | | |
| Skin + | | | | | | | |
| Oral) | | | | | | | |
| 100 ng | No | 1 round | 8 | 80.09 | 18.09 | 94.83 | 27813 |
| ATCC 3 | blockers | 1.3x | | | | | |
| Mix (20 | | QIAseq | | | | | |
| Strain + | | Beads | | | | | |
| Skin + | | | | | | | |
| Oral) | | | | | | | |
| 100 ng | 2.9 | 1 round | 6 | 81.95 | 16.04 | 9.22 | 42471 |
| ATCC 3 | | 1.3x | | | | | |
| Mix (20 | | QIAseq | | | | | |
| Strain + | | Beads | | | | | |
| Skin + | | | | | | | |
| Oral) | | | | | | | |
| 100 ng | 2.9 | 1 round | 4 | 81.55 | 16.54 | 7.69 | 38732 |
| ATCC 3 | | 1.3x | | | | | |
| Mix (20 | | QIAseq | | | | | |
| Strain + | | Beads | | | | | |
| Skin + | | | | | | | |
| Oral) | | | | | | | |
| 100 ng | No | 2 round | 7 | 77.33 | 20.71 | 94.81 | 27649 |
| ATCC 3 | blockers | 1.3x | | | | | |
| Mix (20 | | QIAseq | | | | | |
| Strain + | | | | | | | |
| Beads | | | | | | | |
| Skin + | | | | | | | |
| Oral) | | | | | | | |
| 100 ng | No | 2 round | 8 | 76.73 | 21.32 | 94.71 | 27568 |
| ATCC 3 | blockers | 1.3x | | | | | |
| Mix (20 | | QIAseq | | | | | |
| Strain + | | Beads | | | | | |
| Skin + | | | | | | | |
| Oral) | | | | | | | |
| 100 ng | 2.9 | 2 round | 10 | 83.05 | 14.88 | 16.97 | 47653 |
| ATCC 3 | | 1.3x | | | | | |
| Mix (20 | | QIAseq | | | | | |
| Strain + | | Beads | | | | | |
| Skin + | | | | | | | |
| Oral) | | | | | | | |
| 100 ng | 2.9 | 2 round | 13 | 82.03 | 15.95 | 14.45 | 49602 |
| ATCC 3 | | 1.3x | | | | | |
| Mix (20 | | QIAseq | | | | | |
| Strain + | | Beads | | | | | |
| Skin + | | | | | | | |
| Oral) | | | | | | | |

The results show:

For the ATCC gut sample, 2.9 pmol blocker mix depleted rRNA from about 95% to about 13% or 20%, depending on whether 1 round or 2 rounds of 1.3× bead cleanup are used. Between 1 round and 2 rounds of bead cleanup, the additional round allowed for increased gene detection.

For the ATCC 3 Mix sample (consists of 28 bacterial species when overlapping species are accounted for), 2.9 pmol blocker mix depleted rRNA from about 95% to about 10% or about 15%, depending on whether 1 round or 2 rounds of 1.3× bead cleanup are used. Between 1 round and 2 rounds of bead cleanup, the additional round allowed for increased gene detection. Increasing the amount of blocker mix from 2.9 pmol to 4.35 pmol to 5.8 pmol improved depletion of rRNA.

### Conclusion

The combination of 2.9 pmol of blocker mix and 2 rounds of 1.3× bead cleanup provides the most desirable results when considering both the rRNA depletion and gene expression results.

The results of Examples 5-8 show that for depleting bacterial rRNA, 2.9 pmol of each blocker was the optimal amount with two rounds of bead cleanups. However, for rRNA depletion, 1.45 pmol and even 5.8 pmol of each blocker also worked to deplete rRNA, even with a single round of bead cleanup.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 62/736,006, filed Sep. 25, 2018, which application is hereby incorporated by reference in its entirety.

1. A method for inhibiting cDNA synthesis of one or more unwanted RNA species in an RNA sample during reverse transcription, comprising:
(a) providing an RNA sample that comprises one or more desired RNA species and one or more unwanted RNA species,
(b) annealing one or more blocking oligonucleotides to one or more regions of the one or more unwanted RNA species in the RNA sample to generate a template mixture,
wherein the one or more blocking oligonucleotides are complementary, and stably bind, to the one or more regions of the one or more unwanted RNA species, and comprise 3′ modifications that prevent the one or more blocking oligonucleotides from being extended, and
(c) incubating the template mixture with a reaction mixture that comprises:
(i) at least one reverse transcriptase,
(ii) one or more reverse transcription primers, and
(iii) a reaction buffer,
under conditions sufficient to synthesize cDNA molecules using the one or more desired RNA species as template(s), wherein cDNA synthesis using the one or more unwanted RNA species is inhibited.

2. The method of claim 1, wherein at least one or each of the one or more blocking oligonucleotides comprises one or more modified nucleotides that increase the binding between the one or more blocking oligonucleotides and the regions of the one or more unwanted RNA species.

3. The method of claim 1, wherein at least one or each of the one or more blocking oligonucleotides does not comprise any modified nucleotides that increase the binding between the one or more blocking oligonucleotides and the regions of the one or more unwanted RNA species, and is at least 25 nucleotides long.

4. The method of claim 2, wherein at least one or each of the one or more blocking oligonucleotides comprises one or more locked nucleic acids (LNA).

5. The method of claim 4, wherein the number of LNA in the one or more blocking oligonucleotides ranges from 2 to 20, preferably 4 to 16, more preferably 3 to 15.

6. The method of claim 4, wherein the length of the one or more blocking oligonucleotides ranges from 10 to 30 nucleotides, from 16 to 24 nucleotides, or from 18 to 22 nucleotides.

7. The method of claim 1, wherein the melting temperature (Tm) of duplexes formed between the one or more blocking oligonucleotides and the one or more regions of the one or more unwanted RNA species ranges from 80 to 96° C., or from 86 to 92° C.

8. The method of claim 1, wherein the number of the one or more blocking oligonucleotides is at least 5, at least 10, at least 50, at least 100, at least 150, at least 200, at least 300, at least 400, at least 500, at least 600, at least 700, at least 800, at least 900, at least 1000, at least 1500, or at least 2000, at least 3000, at least 4000, at least 5000, at least 6000, at least 7000, at least 8000, at least 9000, or at least 10,000, and/or
at most 100,000, at most 90,000, at most 80,000, at most 70,000, at most 60,000, or at most 50,000, and/or
from 2 to 100,000, from 100 to 80,000, or from 800 to 50,000.

9. The method of claim 1, wherein the number of the one or more blocking oligonucleotides is at least 5, and wherein two or more of the blocking oligonucleotides anneal to different regions of at least one of the one or more unwanted RNA species.

10. The method of claim 9, wherein the distances between two neighboring regions of the at least one of the one or more unwanted RNA species to which the two or more blocking oligonucleotides anneal range from 0 to 100 nucleotides, 0 to 75 nucleotides, 0 to 50 nucleotides, 20 to 100 nucleotides, 20 to 75 nucleotides, 20 to 50 nucleotides, 30 to 100 nucleotides, 30 to 75 nucleotides, 30 to 50 nucleotides, or 30 to 45 nucleotides.

11. The method of claim 9, wherein the different regions of the at least one of the one or more unwanted RNA species are evenly distributed, and wherein the distances between two neighboring regions range from 20 to 50 nucleotides or from 30 to 45 nucleotides.

12. The method of claim 9, wherein the different regions of the at least one of the one or more unwanted RNA species are not evenly distributed, and wherein the distances between two neighboring regions range from 0 to 100 nucleotides.

13. The method of claim 1, wherein the number of the one or more unwanted RNA species to which the one or more blocking oligonucleotides anneal is at least 2, at least 3, at least 4, or at least 5, at least 10, at least 20, at least 30, at least 40, at least 50, at least 75, at least 100, at least 200, at least 300, at least 400, or at least 500, and/or
at most 1,000,000, at most 500,000, at most 100,000, at most 50,000, at most 10,000, at most 9000, at most 8000, at most 7000, at most 6000, at most 5000, at most 4000, at most 3000, or at most 2000, and/or
from 2 to 1,000,000, from 100 to 500,000, from 500 to 100,000, or from 1000 to 10,000.

14. The method of any of claim 1, wherein the one or more unwanted RNA species comprise rRNA, such as 28S rRNA, 18S rRNA, 5.8S rRNA, 5S rRNA, mitochondrial 12S rRNA, mitochondrial 16S rRNA, and/or plastid rRNA.

15. The method of claim 1, wherein the one or more unwanted RNA species comprise an abundant protein-coding mRNA, tRNA, snoRNA, and/or snRNA.

16. The method of claim 15, wherein the abundant protein-coding mRNA is a globin RNA.

17. The method of claim 1, wherein step (b) is performed in the presence of a salt or KCl.

18. The method of claim 17, wherein the concentration of salt in the template mixture of step (b) ranges from 5 mM to 50 mM, 10 to 30 mM, or 15 mM to 25 mM.

19. The method of claim 1, wherein the amount of each of the one or more blocking oligonucleotides in the template mixture of step (b) ranges from about 0.1 pmol to about 50 pmol per blocking oligonucleotide, from about 0.5 pmol to about 20 pmol, from about 0.5 pmol to about 10 pmol, from about 1 pmol to about 20 pmol, from about 1 pmol to about 10 pmol, from about 1.5 pmol to about 10 pmol, from about 1.5 pmol to about 8 pmol, or from 2 pmol to about 7 pmol per blocking oligonucleotide.

20. The method of claim 1, wherein step (b) comprises:
(i) contacting the one or more blocking oligonucleotides with the RNA sample,
(ii) incubating the mixture of step (i) to at least 65° C., such as at least 70° C. or at least 75° C. for at least 30 second, at least 1 minute, or at least 2 minutes, and
(iii) after step (ii), reducing the temperature to be lower than 40° C., or lower than 25° C.

21. The method of claim 1, wherein the one or more reverse transcription primers are random primers, such as random hexamers.

22. The method of any of claim 1, wherein the RNA sample comprises fragmented RNA molecules.

23. The method of claim 1, wherein the RNA sample is prepared from whole blood, serum, or plasma.

24. The method of claim 1, further comprising:
(d) synthesizing complementary strands of the cDNA molecules generated in step (c) to generate double stranded cDNA molecules.

25. The method of claim 1, further comprising:
(e) amplifying the double stranded cDNA molecules to construct a sequencing library.

26. The method of claim 25, further comprising:
(f) sequencing the one or more desired RNA species using the sequencing library constructed in step (e).

27. The method of claim 1, wherein the one or more blocking oligonucleotides are fully complementary to the one or more regions of the one or more unwanted RNA species.

28. A set of blocking oligonucleotides that are complementary a plurality of regions of an unwanted RNA species, wherein each blocking oligonucleotide comprises one or more modified nucleotides that increase its binding to a region of the unwanted RNA species.

29.-36. (canceled)

37. A plurality of sets of blocking oligonucleotides, wherein each set is according to claim 28.

38.-47. (canceled)

48. A kit of inhibiting cRNA synthesis of one or more unwanted RNA species in an RNA sample, comprising:
(1) (a) one or more blocking oligonucleotides that are complementary to one or more regions of one or more unwanted RNA species in the RNA sample, and each comprise one or more modified nucleotides that increase the binding between the one or more blocking oligonucleotides and the regions of the one or more unwanted RNA species, or
(b) the set or the plurality of sets of blocking oligonucleotides of claim 28, and
(2) a reverse transcriptase.

49. (canceled)

50. A method for designing blocking oligonucleotides for inhibiting cDNA synthesis of one or more unwanted RNA species in an RNA sample during reverse transcription, comprising:
(a) generating multiple blocking oligonucleotides complementary to regions of the one or more unwanted RNA species,
(b) filtering unacceptable blocking oligonucleotides,
(c) generating one or more groups of blocking oligonucleotides that are complementary to multiple different regions of the one or more unwanted RNA species, and
(d) optionally shuffling blocking oligonucleotides among the groups to generate new groups of blocking oligonucleotides, and selecting one or more of the new groups of blocking oligonucleotides.

51.-61. (canceled)


SEQUENCE LISTING (509 sequences)

SEQ ID NO: 1 - DNA, 20 bp, Artificial Sequence
  Forward primer 18S FP2
ctcaacacgggaaacctcac

SEQ ID NO: 2 - DNA, 20 bp, Artificial Sequence
  Reverse primer 18S RP2
cgctccaccaactaagaacg

SEQ ID NO: 3 - DNA, 20 bp, Artificial Sequence
  Forward primer 18S FP1
atggccgttcttagttggtg

SEQ ID NO: 4 - DNA, 20 bp, Artificial Sequence
  Reverse primer 18S RP1
cgctgagccagtcagtgtag

SEQ ID NO: 5 - DNA, 20 bp, Artificial Sequence
  Forward primer 18S FP3
gtaacccgttgaaccccatt

SEQ ID NO: 6 - DNA, 20 bp, Artificial Sequence
  Reverse primer 18S RP3
ccatccaatcggtagtagcg

SEQ ID NO: 7 - DNA, 22 bp, Artificial Sequence
  Forward primer 18S FP4
ggccctgtaattggaatgagtc

SEQ ID NO: 8 - DNA, 21 bp, Artificial Sequence
  Reverse primer 18S RP4
ccaagatccaactacgagctt

SEQ ID NO: 9 - DNA, 20 bp, Artificial Sequence
  Forward primer GAPDH FP
cactgccacccagaagactg

SEQ ID NO: 10 - DNA, 19 bp, Artificial Sequence
  Reverse primer GAPDH RP
cagctcagggatgaccttg

SEQ ID NO: 11 - DNA, 21 bp, Artificial Sequence
  Forward primer ACTB FP
tgcgtgacattaaggagaagc

SEQ ID NO: 12 - DNA, 21 bp, Artificial Sequence
  Reverse primer ACTB RP
ggaaggaaggctggaagagtg

SEQ ID NO: 13 - DNA, 20 bp, Artificial Sequence
  Forward primer RPLP0 FP
caatgttgccagtgtctgtc

SEQ ID NO: 14 - skipped

SEQ ID NO: 15 - DNA, 19 bp, Artificial Sequence
  Synthetic blocker sequence B193
gacaaacccttgtgtcgag

SEQ ID NO: 16 - DNA, 22 bp, Artificial Sequence
  Synthetic blocker sequence B192
agctgctctgctacgtacgaaa

SEQ ID NO: 17 - DNA, 19 bp, Artificial Sequence
  Synthetic blocker sequence B191
gtttagcgccaggttcccc

SEQ ID NO: 18 - DNA, 18 bp, Artificial Sequence
  Synthetic blocker sequence B190
ggccgcctctccggccgc

SEQ ID NO: 19 - DNA, 18 bp, Artificial Sequence
  Synthetic blocker sequence B189
ccggaccccggtcccggc

SEQ ID NO: 20 - DNA, 19 bp, Artificial Sequence
  Synthetic blocker sequence B188
cggggcgcgtggagggggg

SEQ ID NO: 21 - DNA, 18 bp, Artificial Sequence
  Synthetic blocker sequence B187
cggctatccgaggccaac

SEQ ID NO: 22 - DNA, 20 bp, Artificial Sequence
  Synthetic blocker sequence B186
gcctgggcgggattctgact

SEQ ID NO: 23 - DNA, 21 bp, Artificial Sequence
  Synthetic blocker sequence B185
ggtagcttcgccccattggct

SEQ ID NO: 24 - DNA, 19 bp, Artificial Sequence
  Synthetic blocker sequence B184
acctgcggttcctctcgta

SEQ ID NO: 25 - DNA, 21 bp, Artificial Sequence
  Synthetic blocker sequence B183
tcatcagtagggtaaaactaa

SEQ ID NO: 26 - DNA, 20 bp, Artificial Sequence
  Synthetic blocker sequence B182
cgttccctattagtgggtga

SEQ ID NO: 27 - DNA, 18 bp, Artificial Sequence
  Synthetic blocker sequence B181
atgataggaagagccgac

SEQ ID NO: 28 - DNA, 22 bp, Artificial Sequence
  Synthetic blocker sequence B180
tgaacgcttggccgccacaagc

SEQ ID NO: 29 - DNA, 22 bp, Artificial Sequence
  Synthetic blocker sequence B179
acctcctgcttaaaacccaaaa

SEQ ID NO: 30 - DNA, 20 bp, Artificial Sequence
  Synthetic blocker sequence B178
cggtctgtattcgtactgaa

SEQ ID NO: 31 - DNA, 19 bp, Artificial Sequence
  Synthetic blocker sequence B177
ctccacgggaggtttctgt

SEQ ID NO: 32 - DNA, 22 bp, Artificial Sequence
  Synthetic blocker sequence B176
cgttaccgtttgacaggtgtac

SEQ ID NO: 33 - DNA, 18 bp, Artificial Sequence
  Synthetic blocker sequence B175
cccggagcgggtcgcgcc

SEQ ID NO: 34 - DNA, 20 bp, Artificial Sequence
  Synthetic blocker sequence B174
agaagcgagagcccctcggg

SEQ ID NO: 35 - DNA, 19 bp, Artificial Sequence
  Synthetic blocker sequence B173
aaaacgatcagagtagtgg

SEQ ID NO: 36 - DNA, 18 bp, Artificial Sequence
  Synthetic blocker sequence B172
cccgccccgggcccctcg

SEQ ID NO: 37 - DNA, 22 bp, Artificial Sequence
  Synthetic blocker sequence B171
tcccacttattctacacctctc

SEQ ID NO: 38 - DNA, 21 bp, Artificial Sequence
  Synthetic blocker sequence B170
aagctcaacagggtcttcttt

SEQ ID NO: 39 - DNA, 19 bp, Artificial Sequence
  Synthetic blocker sequence B169
gctgtggtttcgctggata

SEQ ID NO: 40 - DNA, 22 bp, Artificial Sequence
  Synthetic blocker sequence B168
atccattcatgcgcgtcactaa

SEQ ID NO: 41 - DNA, 19 bp, Artificial Sequence
  Synthetic blocker sequence B167
gagtcatagttactcccgc

SEQ ID NO: 42 - DNA, 19 bp, Artificial Sequence
  Synthetic blocker sequence B166
tttgacattcagagcactg

SEQ ID NO: 43 - DNA, 19 bp, Artificial Sequence
  Synthetic blocker sequence B165
cgggccttcgcgatgcttt

SEQ ID NO: 44 - DNA, 20 bp, Artificial Sequence
  Synthetic blocker sequence B164
ccgcaccagttctaagtcgg

SEQ ID NO: 45 - DNA, 18 bp, Artificial Sequence
  Synthetic blocker sequence B163
cggaaccgcggccccggg

SEQ ID NO: 46 - DNA, 19 bp, Artificial Sequence
  Synthetic blocker sequence B162
cccctccgccgcctgccgc

SEQ ID NO: 47 - DNA, 19 bp, Artificial Sequence
  Synthetic blocker sequence B161
aacggggggcggacggggc

SEQ ID NO: 48 - DNA, 18 bp, Artificial Sequence
  Synthetic blocker sequence B160
gccccgccgcccgccgac

SEQ ID NO: 49 - DNA, 22 bp, Artificial Sequence
  Synthetic blocker sequence B159
agcggacgcgcgcgcgacgaga

SEQ ID NO: 50 - DNA, 19 bp, Artificial Sequence
  Synthetic blocker sequence B158
cgccgggctccccgggggc

SEQ ID NO: 51 - DNA, 19 bp, Artificial Sequence
  Synthetic blocker sequence B157
cacgggaagggcccggctc

SEQ ID NO: 52 - DNA, 18 bp, Artificial Sequence
  Synthetic blocker sequence B156
gggtgcccgggcccccct

SEQ ID NO: 53 - DNA, 19 bp, Artificial Sequence
  Synthetic blocker sequence B155
ccgcggcgggccgccgccg

SEQ ID NO: 54 - DNA, 17 bp, Artificial Sequence
  Synthetic blocker sequence B154
ccgcccccacgcggcgc

SEQ ID NO: 55 - DNA, 18 bp, Artificial Sequence
  Synthetic blocker sequence B153
ggagagagagagagagag

SEQ ID NO: 56 - DNA, 19 bp, Artificial Sequence
  Synthetic blocker sequence B152
cgcggggtggggcggggga

SEQ ID NO: 57 - DNA, 18 bp, Artificial Sequence
  Synthetic blocker sequence B151
gggcggcggcgcctcgtc

SEQ ID NO: 58 - DNA, 18 bp, Artificial Sequence
  Synthetic blocker sequence B150
ccccagcccgaccgaccc

SEQ ID NO: 59 - DNA, 19 bp, Artificial Sequence
  Synthetic blocker sequence B149
acggatccggcttgccgac

SEQ ID NO: 60 - DNA, 20 bp, Artificial Sequence
  Synthetic blocker sequence B148
gaggctgttcaccttggaga

SEQ ID NO: 61 - DNA, 20 bp, Artificial Sequence
  Synthetic blocker sequence B147
gagatttacaccctctcccc

SEQ ID NO: 62 - DNA, 19 bp, Artificial Sequence
  Synthetic blocker sequence B146
gacgccgccggaaccgcga

SEQ ID NO: 63 - DNA, 18 bp, Artificial Sequence
  Synthetic blocker sequence B145
cgaacccattccagggcg

SEQ ID NO: 64 - DNA, 19 bp, Artificial Sequence
  Synthetic blocker sequence B144
cccggggctcccgccggct

SEQ ID NO: 65 - DNA, 18 bp, Artificial Sequence
  Synthetic blocker sequence B143
gcctcgcggcgcccatct

SEQ ID NO: 66 - DNA, 22 bp, Artificial Sequence
  Synthetic blocker sequence B142
ccgactccctttcgatcggccg

SEQ ID NO: 67 - DNA, 18 bp, Artificial Sequence
  Synthetic blocker sequence B141
aacggcgctcgcccatct

SEQ ID NO: 68 - DNA, 21 bp, Artificial Sequence
  Synthetic blocker sequence B140
ctgttcacatggaacccttct

SEQ ID NO: 69 - DNA, 19 bp, Artificial Sequence
  Synthetic blocker sequence B139
atttgctactaccaccaag

SEQ ID NO: 70 - DNA, 18 bp, Artificial Sequence
  Synthetic blocker sequence B138
cgccctaggcttcaaggc

SEQ ID NO: 71 - DNA, 18 bp, Artificial Sequence
  Synthetic blocker sequence B137
tagcgtccgcggggctcc

SEQ ID NO: 72 - DNA, 18 bp, Artificial Sequence
  Synthetic blocker sequence B136
gggaggaggcgtgggggg

SEQ ID NO: 73 - DNA, 17 bp, Artificial Sequence
  Synthetic blocker sequence B135
cgccgccgccgccgccc

SEQ ID NO: 74 - DNA, 18 bp, Artificial Sequence
  Synthetic blocker sequence B134
ccgcccccgccgctcccg

SEQ ID NO: 75 - DNA, 19 bp, Artificial Sequence
  Synthetic blocker sequence B133
tgggcccgacgctccagcg

SEQ ID NO: 76 - DNA, 22 bp, Artificial Sequence
  Synthetic blocker sequence B132
gcaggtgagttgttacacactc

SEQ ID NO: 77 - DNA, 20 bp, Artificial Sequence
  Synthetic blocker sequence B131
tcctgctgtctatatcaacc

SEQ ID NO: 78 - DNA, 18 bp, Artificial Sequence
  Synthetic blocker sequence B130
atcgggcgccttaacccg

SEQ ID NO: 79 - DNA, 21 bp, Artificial Sequence
  Synthetic blocker sequence B129
tgcttaccaaaagtggcccac

SEQ ID NO: 80 - DNA, 19 bp, Artificial Sequence
  Synthetic blocker sequence B128
ccagcgagccgggcttctt

SEQ ID NO: 81 - DNA, 21 bp, Artificial Sequence
  Synthetic blocker sequence B127
atcgtttcggccccaagacct

SEQ ID NO: 82 - DNA, 19 bp, Artificial Sequence
  Synthetic blocker sequence B126
tggcgggggtgcgtcgggt

SEQ ID NO: 83 - DNA, 19 bp, Artificial Sequence
  Synthetic blocker sequence B125
ttcggagggaaccagctac

SEQ ID NO: 84 - DNA, 21 bp, Artificial Sequence
  Synthetic blocker sequence B124
tacccaggtcggacgaccgat

SEQ ID NO: 85 - DNA, 18 bp, Artificial Sequence
  Synthetic blocker sequence B123
gagtttcctctggcttcg

SEQ ID NO: 86 - DNA, 20 bp, Artificial Sequence
  Synthetic blocker sequence B122
ggtcctaacacgtgcgctcg

SEQ ID NO: 87 - DNA, 18 bp, Artificial Sequence
  Synthetic blocker sequence B121
ggccggtggtgcgccctc

SEQ ID NO: 88 - DNA, 19 bp, Artificial Sequence
  Synthetic blocker sequence B120
cggccggcgagcgcgccgg

SEQ ID NO: 89 - DNA, 19 bp, Artificial Sequence
  Synthetic blocker sequence B119
gtgcgagcccccgactcgc

SEQ ID NO: 90 - DNA, 22 bp, Artificial Sequence
  Synthetic blocker sequence B118
tcaagacgggtcgggtgggtag

SEQ ID NO: 91 - DNA, 18 bp, Artificial Sequence
  Synthetic blocker sequence B117
cgccgtccccctcttcgg

SEQ ID NO: 92 - DNA, 18 bp, Artificial Sequence
  Synthetic blocker sequence B116
ccgggcccgacggcgcga

SEQ ID NO: 93 - DNA, 18 bp, Artificial Sequence
  Synthetic blocker sequence B115
cgccccccgacccgcgcg

SEQ ID NO: 94 - DNA, 18 bp, Artificial Sequence
  Synthetic blocker sequence B114
ggggaggaggggtgggag

SEQ ID NO: 95 - DNA, 18 bp, Artificial Sequence
  Synthetic blocker sequence B113
cccccacgaggagacgcc

SEQ ID NO: 96 - DNA, 18 bp, Artificial Sequence
  Synthetic blocker sequence B112
ggggattccccgcggggg

SEQ ID NO: 97 - DNA, 18 bp, Artificial Sequence
  Synthetic blocker sequence B111
ggtctcgctccctcggcc

SEQ ID NO: 98 - DNA, 21 bp, Artificial Sequence
  Synthetic blocker sequence B110
gggctgtaacactcggggggg

SEQ ID NO: 99 - DNA, 19 bp, Artificial Sequence
  Synthetic blocker sequence B109
caccgccgccgccgccgcc

SEQ ID NO: 100 - skipped

SEQ ID NO: 101 - DNA, 18 bp, Artificial Sequence
  Synthetic blocker sequence B107
gacggggccccccgagcc

SEQ ID NO: 102 - DNA, 19 bp, Artificial Sequence
  Synthetic blocker sequence B106
ggagccggtcgcggcgcac

SEQ ID NO: 103 - DNA, 18 bp, Artificial Sequence
  Synthetic blocker sequence B105
gtcgccggtcgggggacg

SEQ ID NO: 104 - DNA, 18 bp, Artificial Sequence
  Synthetic blocker sequence B104
gcccacccccgcacccgc

SEQ ID NO: 105 - DNA, 18 bp, Artificial Sequence
  Synthetic blocker sequence B103
aggaggaggaggggcggc

SEQ ID NO: 106 - DNA, 22 bp, Artificial Sequence
  Synthetic blocker sequence B102
ggaggaacggggggcgggaaag

SEQ ID NO: 107 - DNA, 18 bp, Artificial Sequence
  Synthetic blocker sequence B101
gccgggttgaatcctccg

SEQ ID NO: 108 - DNA, 22 bp, Artificial Sequence
  Synthetic blocker sequence B100
ctcttaacggtttcacgccctc

SEQ ID NO: 109 - DNA, 19 bp, Artificial Sequence
  Synthetic blocker sequence B99
tcccttacggtacttgttg

SEQ ID NO: 110 - DNA, 22 bp, Artificial Sequence
  Synthetic blocker sequence B98
tagatggagtttaccacccgct

SEQ ID NO: 111 - DNA, 18 bp, Artificial Sequence
  Synthetic blocker sequence B97
aagacccgggcccggcgc

SEQ ID NO: 112 - DNA, 18 bp, Artificial Sequence
  Synthetic blocker sequence B96
gggctgggcctcgatcag

SEQ ID NO: 113 - DNA, 18 bp, Artificial Sequence
  Synthetic blocker sequence B95
agcgggtcttccgtacgc

SEQ ID NO: 114 - DNA, 19 bp, Artificial Sequence
  Synthetic blocker sequence B94
ttcggcgctgggctcttcc

SEQ ID NO: 115 - DNA, 20 bp, Artificial Sequence
  Synthetic blocker sequence B93
gttagtttcttctcctccgc

SEQ ID NO: 116 - DNA, 18 bp, Artificial Sequence
  Synthetic blocker sequence B92
gtctgatctgaggtcgcg

SEQ ID NO: 117 - DNA, 21 bp, Artificial Sequence
  Synthetic blocker sequence B91
cttttacttcctctagatagt

SEQ ID NO: 118 - DNA, 18 bp, Artificial Sequence
  Synthetic blocker sequence B90
gccgtgggccgaccccgg

SEQ ID NO: 119 - DNA, 21 bp, Artificial Sequence
  Synthetic blocker sequence B89
tccaatcggtagtagcgacgg

SEQ ID NO: 120 - DNA, 21 bp, Artificial Sequence
  Synthetic blocker sequence B88
aacgcaagcttatgacccgca

SEQ ID NO: 121 - DNA, 20 bp, Artificial Sequence
  Synthetic blocker sequence B87
attgcaatccccgatcccca

SEQ ID NO: 122 - DNA, 19 bp, Artificial Sequence
  Synthetic blocker sequence B86
tgccggcgtagggtaggca

SEQ ID NO: 123 - DNA, 22 bp, Artificial Sequence
  Synthetic blocker sequence B85
gcagccccggacatctaagggc

SEQ ID NO: 124 - DNA, 18 bp, Artificial Sequence
  Synthetic blocker sequence B84
ctgaacgccacttgtccc

SEQ ID NO: 125 - DNA, 21 bp, Artificial Sequence
  Synthetic blocker sequence B83
ggggtcgcgtaactagttagc

SEQ ID NO: 126 - DNA, 20 bp, Artificial Sequence
  Synthetic blocker sequence B82
ccagacaaatcgctccacca

SEQ ID NO: 127 - DNA, 22 bp, Artificial Sequence
  Synthetic blocker sequence B81
ggaatcgagaaagagctatcaa

SEQ ID NO: 128 - DNA, 22 bp, Artificial Sequence
  Synthetic blocker sequence B80
gtgaggtttcccgtgttgagtc

SEQ ID NO: 129 - DNA, 18 bp, Artificial Sequence
  Synthetic blocker sequence B79
ccttccgtcaattccttt

SEQ ID NO: 130 - DNA, 21 bp, Artificial Sequence
  Synthetic blocker sequence B78
ggaacccaaagactttggttt

SEQ ID NO: 131 - DNA, 19 bp, Artificial Sequence
  Synthetic blocker sequence B77
gccgccgcatcgccggtcg

SEQ ID NO: 132 - DNA, 21 bp, Artificial Sequence
  Synthetic blocker sequence B76
tctgatcgtcttcgaacctcc

SEQ ID NO: 133 - DNA, 19 bp, Artificial Sequence
  Synthetic blocker sequence B75
ggcaaatgctttcgctctg

SEQ ID NO: 134 - DNA, 20 bp, Artificial Sequence
  Synthetic blocker sequence B74
tctagcggcgcaatacgaat

SEQ ID NO: 135 - DNA, 20 bp, Artificial Sequence
  Synthetic blocker sequence B73
agttccgaaaaccaacaaaa

SEQ ID NO: 136 - DNA, 20 bp, Artificial Sequence
  Synthetic blocker sequence B72
ctgcggtatccaggcggctc

SEQ ID NO: 137 - DNA, 20 bp, Artificial Sequence
  Synthetic blocker sequence B71
agtaaacgcttcgggccccg

SEQ ID NO: 138 - DNA, 18 bp, Artificial Sequence
  Synthetic blocker sequence B70
cgagaggcaaggggcggg

SEQ ID NO: 139 - DNA, 18 bp, Artificial Sequence
  Synthetic blocker sequence B69
cgcccgctcccaagatcc

SEQ ID NO: 140 - DNA, 19 bp, Artificial Sequence
  Synthetic blocker sequence B68
tatacgctattggagctgg

SEQ ID NO: 141 - DNA, 21 bp, Artificial Sequence
  Synthetic blocker sequence B67
cctccaatggatcctcgttaa

SEQ ID NO: 142 - DNA, 19 bp, Artificial Sequence
  Synthetic blocker sequence B66
gcctcgaaagagtcctgta

SEQ ID NO: 143 - DNA, 22 bp, Artificial Sequence
  Synthetic blocker sequence B65
tcgggagtgggtaatttgcgcg

SEQ ID NO: 144 - DNA, 20 bp, Artificial Sequence
  Synthetic blocker sequence B64
tctcaggctccctctccgga

SEQ ID NO: 145 - DNA, 20 bp, Artificial Sequence
  Synthetic blocker sequence B63
accatggtaggcacggcgac

SEQ ID NO: 146 - DNA, 18 bp, Artificial Sequence
  Synthetic blocker sequence B62
tgggtcgtcgccgccacg

SEQ ID NO: 147 - DNA, 21 bp, Artificial Sequence
  Synthetic blocker sequence B61
gagtcaccaaagccgccggcg

SEQ ID NO: 148 - DNA, 20 bp, Artificial Sequence
  Synthetic blocker sequence B60
gaccgggttggttttgatct

SEQ ID NO: 149 - DNA, 18 bp, Artificial Sequence
  Synthetic blocker sequence B59
cagcgcccgtcggcatgt

SEQ ID NO: 150 - DNA, 21 bp, Artificial Sequence
  Synthetic blocker sequence B58
gtaggagaggagcgagcgacc

SEQ ID NO: 151 - DNA, 20 bp, Artificial Sequence
  Synthetic Blocker B57
cgcagtttcactgtaccggc

SEQ ID NO: 152 - DNA, 22 bp, Artificial Sequence
  Synthetic Blocker B56
ctttgagacaagcatatgctac

SEQ ID NO: 153 - DNA, 20 bp, Artificial Sequence
  Synthetic Blocker B55
gacaggcgtagccccgggag

SEQ ID NO: 154 - DNA, 22 bp, Artificial Sequence
  Synthetic Blocker B54
gtcgatgatcaatgtgtcctgc

SEQ ID NO: 155 - DNA, 20 bp, Artificial Sequence
  Synthetic Blocker B53
tcttcatcgacgcacgagcc

SEQ ID NO: 156 - DNA, 18 bp, Artificial Sequence
  Synthetic Blocker B52
cttgggtgggtgtgggta

SEQ ID NO: 157 - DNA, 19 bp, Artificial Sequence
  Synthetic Blocker B51
ggaaggcgctttgtgaagt

SEQ ID NO: 158 - DNA, 22 bp, Artificial Sequence
  Synthetic Blocker B50
gggaggaatttgaagtagatag

SEQ ID NO: 159 - DNA, 22 bp, Artificial Sequence
  Synthetic Blocker B49
tcagatcacgtaggactttaat

SEQ ID NO: 160 - DNA, 18 bp, Artificial Sequence
  Synthetic Blocker B48
ccatcgggatgtcctgat

SEQ ID NO: 161 - DNA, 19 bp, Artificial Sequence
  Synthetic Blocker B47
atggactctagaataggat

SEQ ID NO: 162 - DNA, 21 bp, Artificial Sequence
  Synthetic Blocker B46
gttggtcaagttattggatca

SEQ ID NO: 163 - DNA, 22 bp, Artificial Sequence
  Synthetic Blocker B45
gaagtcttagcatgtactgctc

SEQ ID NO: 164 - DNA, 19 bp, Artificial Sequence
  Synthetic Blocker B44
ccgaaatttttaatgcagg

SEQ ID NO: 165 - DNA, 21 bp, Artificial Sequence
  Synthetic Blocker B43
ggtactgtttgcattaataaa

SEQ ID NO: 166 - DNA, 21 bp, Artificial Sequence
  Synthetic Blocker B42
tgtgttatgcccgcctcttca

SEQ ID NO: 167 - DNA, 18 bp, Artificial Sequence
  Synthetic Blocker B41
gacagctgaaccctcgtg

SEQ ID NO: 168 - DNA, 20 bp, Artificial Sequence
  Synthetic Blocker B40
caagtgattatgctaccttt

SEQ ID NO: 169 - DNA, 21 bp, Artificial Sequence
  Synthetic Blocker B39
tgtgtcactgggcaggcggtg

SEQ ID NO: 170 - DNA, 22 bp, Artificial Sequence
  Synthetic Blocker B38
gtttttggtaaacaggcggggt

SEQ ID NO: 171 - DNA, 20 bp, Artificial Sequence
  Synthetic Blocker B37
acctttccttatgagcatgc

SEQ ID NO: 172 - DNA, 22 bp, Artificial Sequence
  Synthetic Blocker B36
tgacttgttggttgattgtaga

SEQ ID NO: 173 - DNA, 19 bp, Artificial Sequence
  Synthetic Blocker B35
aatctgacgcaggcttatg

SEQ ID NO: 174 - DNA, 20 bp, Artificial Sequence
  Synthetic Blocker B34
aacattagttcttctatagg

SEQ ID NO: 175 - DNA, 22 bp, Artificial Sequence
  Synthetic Blocker B33
agttcagttatatgtttgggat

SEQ ID NO: 176 - DNA, 22 bp, Artificial Sequence
  Synthetic Blocker B32
gctttcttaattggtggctgct

SEQ ID NO: 177 - DNA, 21 bp, Artificial Sequence
  Synthetic Blocker B31
actctctctacaaggtttttt

SEQ ID NO: 178 - DNA, 22 bp, Artificial Sequence
  Synthetic Blocker B30
gactaacagttaaatttacaag

SEQ ID NO: 179 - DNA, 19 bp, Artificial Sequence
  Synthetic Blocker B29
gttgaactaagattctatc

SEQ ID NO: 180 - DNA, 20 bp, Artificial Sequence
  Synthetic Blocker B28
gtttgtcgcctctacctata

SEQ ID NO: 181 - DNA, 22 bp, Artificial Sequence
  Synthetic Blocker B27
ggtgtgctcttttagctgttct

SEQ ID NO: 182 - DNA, 18 bp, Artificial Sequence
  Synthetic Blocker B26
ttggctctccttgcaaag

SEQ ID NO: 183 - DNA, 20 bp, Artificial Sequence
  Synthetic Blocker B25
ataggggttagtccttgcta

SEQ ID NO: 184 - DNA, 20 bp, Artificial Sequence
  Synthetic Blocker B24
ccttgcggtactatatctat

SEQ ID NO: 185 - DNA, 22 bp, Artificial Sequence
  Synthetic Blocker B23
actttatttgggtaaatggttt

SEQ ID NO: 186 - DNA, 22 bp, Artificial Sequence
  Synthetic Blocker B22
tgggtttggggctaggtttagc

SEQ ID NO: 187 - DNA, 19 bp, Artificial Sequence
  Synthetic Blocker B21
ttacgacttgtctcctcta

SEQ ID NO: 188 - DNA, 22 bp, Artificial Sequence
  Synthetic Blocker B20
tcctttgaagtatacttgagga

SEQ ID NO: 189 - DNA, 20 bp, Artificial Sequence
  Synthetic Blocker B19
ccctgttcaactaagcactc

SEQ ID NO: 190 - DNA, 22 bp, Artificial Sequence
  Synthetic Blocker B18
cgacccttaagtttcataaggg

SEQ ID NO: 191 - DNA, 19 bp, Artificial Sequence
  Synthetic Blocker B17
ccatttcttgccacctcat

SEQ ID NO: 192 - DNA, 19 bp, Artificial Sequence
  Synthetic Blocker B16
gtacttgcgcttactttgt

SEQ ID NO: 193 - DNA, 22 bp, Artificial Sequence
  Synthetic Blocker B15
ggtatataggctgagcaagagg

SEQ ID NO: 194 - DNA, 20 bp, Artificial Sequence
  Synthetic Blocker B14
gaacaggctcctctagaggg

SEQ ID NO: 195 - DNA, 19 bp, Artificial Sequence
  Synthetic Blocker B13
agctgtggctcgtagtgtt

SEQ ID NO: 196 - DNA, 21 bp, Artificial Sequence
  Synthetic Blocker B12
gaggtttagggctaagcatag

SEQ ID NO: 197 - DNA, 22 bp, Artificial Sequence
  Synthetic Blocker B11
gctattgtgtgttcagatatgt

SEQ ID NO: 198 - DNA, 22 bp, Artificial Sequence
  Synthetic Blocker B10
caactggagttttttacaactc

SEQ ID NO: 199 - DNA, 20 bp, Artificial Sequence
  Synthetic Blocker B9
acactctttacgccggcttc

SEQ ID NO: 200 - DNA, 22 bp, Artificial Sequence
  Synthetic Blocker B8
ggtggctggcacgaaattgacc

SEQ ID NO: 201 - DNA, 22 bp, Artificial Sequence
  Synthetic Blocker B7
actttcgtttattgctaaaggt

SEQ ID NO: 202 - DNA, 21 bp, Artificial Sequence
  Synthetic Blocker B6
gctaggctaagcgttttgagc

SEQ ID NO: 203 - DNA, 22 bp, Artificial Sequence
  Synthetic Blocker B5
cttttgatcgtggtgatttaga

SEQ ID NO: 204 - DNA, 18 bp, Artificial Sequence
  Synthetic Blocker B4
gtgtaatcttactaagag

SEQ ID NO: 205 - DNA, 19 bp, Artificial Sequence
  Synthetic Blocker B3
agcctacagcacccggtat

SEQ ID NO: 206 - DNA, 18 bp, Artificial Sequence
  Synthetic Blocker B2
ggcccgaccctgcttagc

SEQ ID NO: 207 - DNA, 18 bp, Artificial Sequence
  Synthetic Blocker B1
ggtggtatggccgtagac

SEQ ID NO: 208 - DNA, 20 bp, Artificial Sequence
  Synthetic bloker sequence 5S1
cgtttcacttctgagttcgg

SEQ ID NO: 209 - DNA, 20 bp, Artificial Sequence
  Synthetic bloker sequence 5S2
accccacactaccatcggcg

SEQ ID NO: 210 - DNA, 20 bp, Artificial Sequence
  Synthetic bloker sequence 5S3
cttagcttccgggttcggaa

SEQ ID NO: 211 - DNA, 20 bp, Artificial Sequence
  Synthetic bloker sequence 5S4
gtgttcgggatgggaacggg

SEQ ID NO: 212 - DNA, 20 bp, Artificial Sequence
  Synthetic bloker sequence 5S5
cgagttcgggatgggatcgg

SEQ ID NO: 213 - DNA, 20 bp, Artificial Sequence
  Synthetic bloker sequence 5S6
tctgttcggaatgggaagag

SEQ ID NO: 214 - DNA, 20 bp, Artificial Sequence
  Synthetic bloker sequence 5S7
agcttaacttctgtgttcgg

SEQ ID NO: 215 - DNA, 20 bp, Artificial Sequence
  Synthetic bloker sequence 5S8
agcttaacttccgtgttcgg

SEQ ID NO: 216 - DNA, 20 bp, Artificial Sequence
  Synthetic bloker sequence 5S9
tcctgttcgggatgggaagg

SEQ ID NO: 217 - DNA, 20 bp, Artificial Sequence
  Synthetic bloker sequence 5S10
ggcggtgtcctactctccca

SEQ ID NO: 218 - DNA, 20 bp, Artificial Sequence
  Synthetic bloker sequence 5S11
gtgttcggaatgggaacggg

SEQ ID NO: 219 - DNA, 20 bp, Artificial Sequence
  Synthetic bloker sequence 5S12
ccccaactaccatcggcgct

SEQ ID NO: 220 - DNA, 20 bp, Artificial Sequence
  Synthetic bloker sequence 5S13
atgacctactctcacatggg

SEQ ID NO: 221 - DNA, 20 bp, Artificial Sequence
  Synthetic bloker sequence 5S14
actctcgcatggggagaccc

SEQ ID NO: 222 - DNA, 20 bp, Artificial Sequence
  Synthetic bloker sequence 5S15
ggcggcgtcctactctccca

SEQ ID NO: 223 - DNA, 20 bp, Artificial Sequence
  Synthetic bloker sequence 5S16
gtgcagtaccatcggcgctg

SEQ ID NO: 224 - DNA, 20 bp, Artificial Sequence
  Synthetic bloker sequence 5S17
ccgagttcggaatgggatcg

SEQ ID NO: 225 - DNA, 20 bp, Artificial Sequence
  Synthetic bloker sequence 5S18
tggcagcgacctactctccc

SEQ ID NO: 226 - DNA, 20 bp, Artificial Sequence
  Synthetic bloker sequence 5S19
tgtcctactctcacatgggg

SEQ ID NO: 227 - DNA, 20 bp, Artificial Sequence
  Synthetic bloker sequence 5S20
ggcggcgacctactctccca

SEQ ID NO: 228 - DNA, 20 bp, Artificial Sequence
  Synthetic bloker sequence 5S21
gagttcgggatgggatcagg

SEQ ID NO: 229 - DNA, 20 bp, Artificial Sequence
  Synthetic bloker sequence 5S22
gtcctactctcacaggggga

SEQ ID NO: 230 - DNA, 20 bp, Artificial Sequence
  Synthetic bloker sequence 5S23
ctgcagtaccatcggcgctg

SEQ ID NO: 231 - DNA, 20 bp, Artificial Sequence
  Synthetic bloker sequence 5S24
cgggttcgggatgggaccgg

SEQ ID NO: 232 - DNA, 20 bp, Artificial Sequence
  Synthetic bloker sequence 5S25
agtaccatcggcgctggagg

SEQ ID NO: 233 - DNA, 20 bp, Artificial Sequence
  Synthetic bloker sequence 5S26
ctgtgttcggcatgggaaca

SEQ ID NO: 234 - DNA, 20 bp, Artificial Sequence
  Synthetic bloker sequence 5S27
gcctggcaacgtcctactct

SEQ ID NO: 235 - DNA, 20 bp, Artificial Sequence
  Synthetic bloker sequence 5S28
tgacgatgacctactttcac

SEQ ID NO: 236 - DNA, 20 bp, Artificial Sequence
  Synthetic bloker sequence 5S29
gtgttcgggatgggaacagg

SEQ ID NO: 237 - DNA, 20 bp, Artificial Sequence
  Synthetic bloker sequence 5S30
tgcctggcagttccctactc

SEQ ID NO: 238 - DNA, 20 bp, Artificial Sequence
  Synthetic bloker sequence 5S31
ggcggtgacctactctccca

SEQ ID NO: 239 - DNA, 20 bp, Artificial Sequence
  Synthetic bloker sequence 5S32
tgttcggaatgggaacaggt

SEQ ID NO: 240 - DNA, 20 bp, Artificial Sequence
  Synthetic bloker sequence 5S33
ccgagttcgagatgggatcg

SEQ ID NO: 241 - DNA, 20 bp, Artificial Sequence
  Synthetic bloker sequence 5S34
ggcaacgacctactctccca

SEQ ID NO: 242 - DNA, 20 bp, Artificial Sequence
  Synthetic bloker sequence 5S35
cagggggcaacccccaacta

SEQ ID NO: 243 - DNA, 20 bp, Artificial Sequence
  Synthetic bloker sequence 5S36
accatcggcgctgaagagct

SEQ ID NO: 244 - DNA, 20 bp, Artificial Sequence
  Synthetic bloker sequence 5S37
aatccgcactatcatcggcg

SEQ ID NO: 245 - DNA, 20 bp, Artificial Sequence
  Synthetic bloker sequence 5S38
ggcggcgacctactctcccg

SEQ ID NO: 246 - DNA, 20 bp, Artificial Sequence
  Synthetic bloker sequence 5S39
ttcggcatgggaacgggtgt

SEQ ID NO: 247 - DNA, 20 bp, Artificial Sequence
  Synthetic bloker sequence 5S40
gggcttaacttctctgttcg

SEQ ID NO: 248 - DNA, 20 bp, Artificial Sequence
  Synthetic bloker sequence 5S41
cacaccgtctccagtgcagt

SEQ ID NO: 249 - DNA, 20 bp, Artificial Sequence
  Synthetic bloker sequence 5S42
gttcggcggtgtcctacttt

SEQ ID NO: 250 - DNA, 20 bp, Artificial Sequence
  Synthetic bloker sequence 5S43
cggcagcgacctactctccc

SEQ ID NO: 251 - DNA, 20 bp, Artificial Sequence
  Synthetic bloker sequence 5S44
tcccaactaccatcggcgct

SEQ ID NO: 252 - DNA, 20 bp, Artificial Sequence
  Synthetic bloker sequence 5S45
gggttcggaatgggaccggg

SEQ ID NO: 253 - DNA, 20 bp, Artificial Sequence
  Synthetic bloker sequence 5S46
actctcacatggggagaccc

SEQ ID NO: 254 - DNA, 20 bp, Artificial Sequence
  Synthetic bloker sequence 5S47
acgcagtaccatcggcgtga

SEQ ID NO: 255 - DNA, 20 bp, Artificial Sequence
  Synthetic bloker sequence 5S48
gattacctactttcacacgg

SEQ ID NO: 256 - DNA, 20 bp, Artificial Sequence
  Synthetic bloker sequence 5S49
gcggctacctactctcccac

SEQ ID NO: 257 - DNA, 20 bp, Artificial Sequence
  Synthetic bloker sequence 5S50
ttcggcatgggtacaggtgt

SEQ ID NO: 258 - DNA, 20 bp, Artificial Sequence
  Synthetic bloker sequence 5S51
ctgagttcggcatggggtca

SEQ ID NO: 259 - DNA, 20 bp, Artificial Sequence
  Synthetic bloker sequence 5S52
tggcgacgtcctactctcac

SEQ ID NO: 260 - DNA, 20 bp, Artificial Sequence
  Synthetic bloker sequence 5S53
acacagtctcccatgcagta

SEQ ID NO: 261 - DNA, 20 bp, Artificial Sequence
  Synthetic bloker sequence 5S54
ctgtgttcggtatgggaaca

SEQ ID NO: 262 - DNA, 20 bp, Artificial Sequence
  Synthetic bloker sequence 5S55
cgatgacctactctcgcatg

SEQ ID NO: 263 - DNA, 20 bp, Artificial Sequence
  Synthetic bloker sequence 5S56
gtgcagtaccatcggcgcag

SEQ ID NO: 264 - DNA, 20 bp, Artificial Sequence
  Synthetic bloker sequence 5S57
ggcgacgacctactctccca

SEQ ID NO: 265 - DNA, 20 bp, Artificial Sequence
  Synthetic bloker sequence 5S58
ttcggcatgggatcaggtgg

SEQ ID NO: 266 - DNA, 20 bp, Artificial Sequence
  Synthetic bloker sequence 5S59
tggcagcgacttactctccc

SEQ ID NO: 267 - DNA, 20 bp, Artificial Sequence
  Synthetic bloker sequence 5S60
tcctgttcggaatgggaagg

SEQ ID NO: 268 - DNA, 20 bp, Artificial Sequence
  Synthetic bloker sequence 5S61
cctggcgatgacctactttc

SEQ ID NO: 269 - DNA, 20 bp, Artificial Sequence
  Synthetic bloker sequence 5S62
gagttcggaatgggatcagg

SEQ ID NO: 270 - DNA, 20 bp, Artificial Sequence
  Synthetic bloker sequence 5S63
tgagttcgggaagggatcag

SEQ ID NO: 271 - DNA, 20 bp, Artificial Sequence
  Synthetic bloker sequence 5S64
ccacactatcatcggcgcta

SEQ ID NO: 272 - DNA, 20 bp, Artificial Sequence
  Synthetic bloker sequence 5S65
gtgtgacctctctgccatca

SEQ ID NO: 273 - DNA, 20 bp, Artificial Sequence
  Synthetic bloker sequence 5S66
ttcggtatgggaacgggtgt

SEQ ID NO: 274 - DNA, 20 bp, Artificial Sequence
  Synthetic bloker sequence 5S67
tcgtgttcgggatgggtacg

SEQ ID NO: 275 - DNA, 20 bp, Artificial Sequence
  Synthetic bloker sequence 5S68
cccggcaacgtcctactctc

SEQ ID NO: 276 - DNA, 20 bp, Artificial Sequence
  Synthetic bloker sequence 5S69
gcgctggagcgtttcacggc

SEQ ID NO: 277 - DNA, 20 bp, Artificial Sequence
  Synthetic bloker sequence 5S70
cgctggggcgtttcacggcc

SEQ ID NO: 278 - DNA, 20 bp, Artificial Sequence
  Synthetic bloker sequence 5S71
tactctcacatggggaaacc

SEQ ID NO: 279 - DNA, 20 bp, Artificial Sequence
  Synthetic bloker sequence 5S72
ttccctcacgctatgaccac

SEQ ID NO: 280 - DNA, 20 bp, Artificial Sequence
  Synthetic bloker sequence 5S73
attgcagtaccatcggcgca

SEQ ID NO: 281 - DNA, 20 bp, Artificial Sequence
  Synthetic bloker sequence 5S74
ccacactatcatcggcgctg

SEQ ID NO: 282 - DNA, 20 bp, Artificial Sequence
  Synthetic bloker sequence 5S75
aggaccctgcggtccaagta

SEQ ID NO: 283 - DNA, 20 bp, Artificial Sequence
  Synthetic bloker sequence 5S76
acctggcggcgaccgacttt

SEQ ID NO: 284 - DNA, 20 bp, Artificial Sequence
  Synthetic bloker sequence 5S77
gtgcagtaccatcgccgtgc

SEQ ID NO: 285 - DNA, 20 bp, Artificial Sequence
  Synthetic bloker sequence 5S78
gccccacactaccatcggcg

SEQ ID NO: 286 - DNA, 20 bp, Artificial Sequence
  Synthetic bloker sequence 5S79
cacttctgagttcgagatgg

SEQ ID NO: 287 - DNA, 20 bp, Artificial Sequence
  Synthetic bloker sequence 5S80
cctactctcccgcattgcat

SEQ ID NO: 288 - DNA, 20 bp, Artificial Sequence
  Synthetic bloker sequence 5S81
gttcgagatgggaacaggtg

SEQ ID NO: 289 - DNA, 20 bp, Artificial Sequence
  Synthetic bloker sequence 5S82
accatcggcgctaaagagct

SEQ ID NO: 290 - DNA, 20 bp, Artificial Sequence
  Synthetic bloker sequence 5S83
gggcagtatcatcggcgctg

SEQ ID NO: 291 - DNA, 20 bp, Artificial Sequence
  Synthetic bloker sequence 5S84
ctggcgacgacctactcttc

SEQ ID NO: 292 - DNA, 20 bp, Artificial Sequence
  Synthetic bloker sequence 5S85
tcgagttcgggatgggatcg

SEQ ID NO: 293 - DNA, 20 bp, Artificial Sequence
  Synthetic bloker sequence 5S86
gccacactaccatcggcgct

SEQ ID NO: 294 - DNA, 20 bp, Artificial Sequence
  Synthetic bloker sequence 5S87
gcagctgcgtttcacttccg

SEQ ID NO: 295 - DNA, 20 bp, Artificial Sequence
  Synthetic bloker sequence 5S88
catagtaccattagcgctat

SEQ ID NO: 296 - DNA, 20 bp, Artificial Sequence
  Synthetic bloker sequence 5S89
accatcggcgcaaaagagct

SEQ ID NO: 297 - DNA, 20 bp, Artificial Sequence
  Synthetic bloker sequence 5S90
ctgtgttcgacatgggaaca

SEQ ID NO: 298 - DNA, 20 bp, Artificial Sequence
  Synthetic bloker sequence 5S91
ggcgacgacctactctcccg

SEQ ID NO: 299 - DNA, 20 bp, Artificial Sequence
  Synthetic bloker sequence 5S92
ggcgacgtcctactctccca

SEQ ID NO: 300 - DNA, 20 bp, Artificial Sequence
  Synthetic bloker sequence 5S93
aacgctatggtcgccaagca

SEQ ID NO: 301 - DNA, 20 bp, Artificial Sequence
  Synthetic bloker sequence 5S94
tgcctggcagtgtcctactc

SEQ ID NO: 302 - DNA, 20 bp, Artificial Sequence
  Synthetic bloker sequence 5S95
ggcgactacctactctccca

SEQ ID NO: 303 - DNA, 20 bp, Artificial Sequence
  Synthetic bloker sequence 5S96
cggcgctaagaagcttaact

SEQ ID NO: 304 - DNA, 20 bp, Artificial Sequence
  Synthetic bloker sequence 5S97
gggcttaactgctgtgttcg

SEQ ID NO: 305 - DNA, 20 bp, Artificial Sequence
  Synthetic bloker sequence 5S98
gtgctactctcccacaccct

SEQ ID NO: 306 - DNA, 20 bp, Artificial Sequence
  Synthetic bloker sequence 5S99
ggcaacgtcctactctccca

SEQ ID NO: 307 - DNA, 20 bp, Artificial Sequence
  Synthetic bloker sequence 5S100
gtcctactctcgcaggggga

SEQ ID NO: 308 - DNA, 20 bp, Artificial Sequence
  Synthetic bloker sequence 16S1
ctgctgcctcccgtaggagt

SEQ ID NO: 309 - DNA, 20 bp, Artificial Sequence
  Synthetic bloker sequence 16S2
gtattaccgcggctgctggc

SEQ ID NO: 310 - DNA, 20 bp, Artificial Sequence
  Synthetic bloker sequence 16S3
actaccagggtatctaatcc

SEQ ID NO: 311 - DNA, 20 bp, Artificial Sequence
  Synthetic bloker sequence 16S4
gctcgttgcgggacttaacc

SEQ ID NO: 312 - DNA, 20 bp, Artificial Sequence
  Synthetic bloker sequence 16S5
cccgtcaattcctttgagtt

SEQ ID NO: 313 - DNA, 20 bp, Artificial Sequence
  Synthetic bloker sequence 16S6
tgacgggcggtgtgtacaag

SEQ ID NO: 314 - DNA, 20 bp, Artificial Sequence
  Synthetic bloker sequence 16S7
tgacgtcatccccaccttcc

SEQ ID NO: 315 - DNA, 20 bp, Artificial Sequence
  Synthetic bloker sequence 16S8
ggtaaggttcttcgcgttgc

SEQ ID NO: 316 - DNA, 20 bp, Artificial Sequence
  Synthetic bloker sequence 16S9
cgagctgacgacagccatgc

SEQ ID NO: 317 - DNA, 20 bp, Artificial Sequence
  Synthetic bloker sequence 16S10
ttgtagcacgtgtgtagccc

SEQ ID NO: 318 - DNA, 20 bp, Artificial Sequence
  Synthetic bloker sequence 16S11
cacatgctccaccgcttgtg

SEQ ID NO: 319 - DNA, 20 bp, Artificial Sequence
  Synthetic bloker sequence 16S12
tctacgcatttcaccgctac

SEQ ID NO: 320 - DNA, 20 bp, Artificial Sequence
  Synthetic bloker sequence 16S13
atcgtttacggcgtggacta

SEQ ID NO: 321 - DNA, 20 bp, Artificial Sequence
  Synthetic bloker sequence 16S14
ctttacgcccagtaattccg

SEQ ID NO: 322 - DNA, 20 bp, Artificial Sequence
  Synthetic bloker sequence 16S15
cgagctgacgacaaccatgc

SEQ ID NO: 323 - DNA, 20 bp, Artificial Sequence
  Synthetic bloker sequence 16S16
cgccttcgccactggtgttc

SEQ ID NO: 324 - DNA, 20 bp, Artificial Sequence
  Synthetic bloker sequence 16S17
ttactagcgattccgacttc

SEQ ID NO: 325 - DNA, 20 bp, Artificial Sequence
  Synthetic bloker sequence 16S18
cgttcgacttgcatgtgtta

SEQ ID NO: 326 - DNA, 20 bp, Artificial Sequence
  Synthetic bloker sequence 16S19
accttgttacgacttcaccc

SEQ ID NO: 327 - DNA, 20 bp, Artificial Sequence
  Synthetic bloker sequence 16S20
ccattgtgcaatattcccca

SEQ ID NO: 328 - DNA, 20 bp, Artificial Sequence
  Synthetic bloker sequence 16S21
tttacaacccgaaggccttc

SEQ ID NO: 329 - DNA, 20 bp, Artificial Sequence
  Synthetic bloker sequence 16S22
ctgagccaggatcaaactct

SEQ ID NO: 330 - DNA, 20 bp, Artificial Sequence
  Synthetic bloker sequence 16S23
tcatcctctcagaccagcta

SEQ ID NO: 331 - DNA, 20 bp, Artificial Sequence
  Synthetic bloker sequence 16S24
ttactcacccgtccgccgct

SEQ ID NO: 332 - DNA, 20 bp, Artificial Sequence
  Synthetic bloker sequence 16S25
ttactcacccgttcgccact

SEQ ID NO: 333 - DNA, 20 bp, Artificial Sequence
  Synthetic bloker sequence 16S26
ttactcacccgtccgccact

SEQ ID NO: 334 - DNA, 20 bp, Artificial Sequence
  Synthetic bloker sequence 16S27
tacctcaccaactagctaat

SEQ ID NO: 335 - DNA, 20 bp, Artificial Sequence
  Synthetic bloker sequence 16S28
gccgtactccccaggcggtc

SEQ ID NO: 336 - DNA, 20 bp, Artificial Sequence
  Synthetic bloker sequence 16S29
cgcgattactagcgattcca

SEQ ID NO: 337 - DNA, 20 bp, Artificial Sequence
  Synthetic bloker sequence 16S30
cccgggaacgtattcaccgc

SEQ ID NO: 338 - DNA, 20 bp, Artificial Sequence
  Synthetic bloker sequence 16S31
ccattgtccaatattcccca

SEQ ID NO: 339 - DNA, 20 bp, Artificial Sequence
  Synthetic bloker sequence 16S32
cgctcgacttgcatgtgtta

SEQ ID NO: 340 - DNA, 20 bp, Artificial Sequence
  Synthetic bloker sequence 16S33
ctttacgcccaataattccg

SEQ ID NO: 341 - DNA, 20 bp, Artificial Sequence
  Synthetic blocker sequence 16S34
tttgagttttaaccttgcgg

SEQ ID NO: 342 - DNA, 20 bp, Artificial Sequence
  Synthetic blocker sequence 16S35
ttcccaggttgagcccgggg

SEQ ID NO: 343 - DNA, 20 bp, Artificial Sequence
  Synthetic blocker sequence 16S36
taccccaccaactagctaat

SEQ ID NO: 344 - DNA, 20 bp, Artificial Sequence
  Synthetic blocker sequence 16S37
tgacgtcgtccccaccttcc

SEQ ID NO: 345 - DNA, 20 bp, Artificial Sequence
  Synthetic blocker sequence 16S38
cacgcggcgtcgctgcatca

SEQ ID NO: 346 - DNA, 20 bp, Artificial Sequence
  Synthetic blocker sequence 16S39
ctcagtcccagtgtggctga

SEQ ID NO: 347 - DNA, 20 bp, Artificial Sequence
  Synthetic blocker sequence 16S40
tcaccctctcaggtcggcta

SEQ ID NO: 348 - DNA, 20 bp, Artificial Sequence
  Synthetic blocker sequence 16S41
tgcagactccaatccggact

SEQ ID NO: 349 - DNA, 20 bp, Artificial Sequence
  Synthetic blocker sequence 16S42
cacgcggcatggctggatca

SEQ ID NO: 350 - DNA, 20 bp, Artificial Sequence
  Synthetic blocker sequence 16S43
acccactcccatggtgtgac

SEQ ID NO: 351 - DNA, 20 bp, Artificial Sequence
  Synthetic blocker sequence 16S44
tacgaatttcacctctacac

SEQ ID NO: 352 - DNA, 20 bp, Artificial Sequence
  Synthetic blocker sequence 16S45
atcgtttagggcgtggacta

SEQ ID NO: 353 - DNA, 20 bp, Artificial Sequence
  Synthetic blocker sequence 16S46
cgtactccccaggcggagtg

SEQ ID NO: 354 - DNA, 20 bp, Artificial Sequence
  Synthetic blocker sequence 16S47
cgccttcgccaccggtgttc

SEQ ID NO: 355 - DNA, 20 bp, Artificial Sequence
  Synthetic blocker sequence 16S48
gccgtactccccaggcgggg

SEQ ID NO: 356 - DNA, 20 bp, Artificial Sequence
  Synthetic blocker sequence 16S49
ccctctcaggccggctaccc

SEQ ID NO: 357 - DNA, 20 bp, Artificial Sequence
  Synthetic blocker sequence 16S50
gtcaggctttcgcccattgc

SEQ ID NO: 358 - DNA, 20 bp, Artificial Sequence
  Synthetic blocker sequence 16S51
ggtaaggttctgcgcgttgc

SEQ ID NO: 359 - DNA, 20 bp, Artificial Sequence
  Synthetic blocker sequence 16S52
ctttcgctcctcagcgtcag

SEQ ID NO: 360 - DNA, 20 bp, Artificial Sequence
  Synthetic blocker sequence 16S53
ctttcgcgcctcagcgtcag

SEQ ID NO: 361 - DNA, 20 bp, Artificial Sequence
  Synthetic blocker sequence 16S54
tatcatcgaattaaaccaca

SEQ ID NO: 362 - DNA, 20 bp, Artificial Sequence
  Synthetic blocker sequence 16S55
tttacaacccgaaggccgtc

SEQ ID NO: 363 - DNA, 20 bp, Artificial Sequence
  Synthetic blocker sequence 16S56
atccgaactgagaccggctt

SEQ ID NO: 364 - DNA, 20 bp, Artificial Sequence
  Synthetic blocker sequence 16S57
tacgcatttcactgctacac

SEQ ID NO: 365 - DNA, 20 bp, Artificial Sequence
  Synthetic blocker sequence 16S58
ggtaaggttcctcgcgtatc

SEQ ID NO: 366 - DNA, 20 bp, Artificial Sequence
  Synthetic blocker sequence 16S59
caccgctacaccaggaattc

SEQ ID NO: 367 - DNA, 20 bp, Artificial Sequence
  Synthetic blocker sequence 16S60
cgccttcgccaccggtattc

SEQ ID NO: 368 - DNA, 20 bp, Artificial Sequence
  Synthetic blocker sequence 16S61
aaggggcatgatgatttgac

SEQ ID NO: 369 - DNA, 20 bp, Artificial Sequence
  Synthetic blocker sequence 16S62
atgctccgccgcttgtgcgg

SEQ ID NO: 370 - DNA, 20 bp, Artificial Sequence
  Synthetic blocker sequence 16S63
ctcagttccagtgtggctgg

SEQ ID NO: 371 - DNA, 20 bp, Artificial Sequence
  Synthetic blocker sequence 16S64
tgcatcaggcttgcgcccat

SEQ ID NO: 372 - DNA, 20 bp, Artificial Sequence
  Synthetic blocker sequence 16S65
taaatccggataacgcttgc

SEQ ID NO: 373 - DNA, 20 bp, Artificial Sequence
  Synthetic blocker sequence 16S66
ccaacatctcacgacacgag

SEQ ID NO: 374 - DNA, 20 bp, Artificial Sequence
  Synthetic blocker sequence 16S67
caccaacaagctgataggcc

SEQ ID NO: 375 - DNA, 20 bp, Artificial Sequence
  Synthetic blocker sequence 16S68
ctcagtcccaatgtggccgt

SEQ ID NO: 376 - DNA, 20 bp, Artificial Sequence
  Synthetic blocker sequence 16S69
ccaccgcttgtgcgggtccc

SEQ ID NO: 377 - DNA, 20 bp, Artificial Sequence
  Synthetic blocker sequence 16S70
tgccttcgccatcggtgttc

SEQ ID NO: 378 - DNA, 20 bp, Artificial Sequence
  Synthetic blocker sequence 16S71
atcgtttacagcgtggacta

SEQ ID NO: 379 - DNA, 20 bp, Artificial Sequence
  Synthetic blocker sequence 16S72
tcactcacgcggcgttgctc

SEQ ID NO: 380 - DNA, 20 bp, Artificial Sequence
  Synthetic blocker sequence 16S73
ttcgcgttgcatcgaattaa

SEQ ID NO: 381 - DNA, 20 bp, Artificial Sequence
  Synthetic blocker sequence 16S74
ctcagtcccagtgtggccgg

SEQ ID NO: 382 - DNA, 20 bp, Artificial Sequence
  Synthetic blocker sequence 16S75
aagggccatgaggacttgac

SEQ ID NO: 383 - DNA, 20 bp, Artificial Sequence
  Synthetic blocker sequence 16S76
gctttcgcacctcagcgtca

SEQ ID NO: 384 - DNA, 20 bp, Artificial Sequence
  Synthetic blocker sequence 16S77
tcgacttgcatgtattaggc

SEQ ID NO: 385 - DNA, 20 bp, Artificial Sequence
  Synthetic blocker sequence 16S78
taaggggcatgatgacttga

SEQ ID NO: 386 - DNA, 20 bp, Artificial Sequence
  Synthetic blocker sequence 16S79
ctgagccatgatcaaactct

SEQ ID NO: 387 - DNA, 20 bp, Artificial Sequence
  Synthetic blocker sequence 16S80
ggggtcgagttgcagacccc

SEQ ID NO: 388 - DNA, 20 bp, Artificial Sequence
  Synthetic blocker sequence 16S81
ttgtccaaaattccccactg

SEQ ID NO: 389 - DNA, 20 bp, Artificial Sequence
  Synthetic blocker sequence 16S82
ctgcgattactagcgactcc

SEQ ID NO: 390 - DNA, 20 bp, Artificial Sequence
  Synthetic blocker sequence 16S83
gcaccaatccatctctggaa

SEQ ID NO: 391 - DNA, 20 bp, Artificial Sequence
  Synthetic blocker sequence 16S84
cgctccctttacacccagta

SEQ ID NO: 392 - DNA, 20 bp, Artificial Sequence
  Synthetic blocker sequence 16S85
taaggacaagggttgcgctc

SEQ ID NO: 393 - DNA, 20 bp, Artificial Sequence
  Synthetic blocker sequence 16S86
tgcagactgcgatccggact

SEQ ID NO: 394 - DNA, 20 bp, Artificial Sequence
  Synthetic blocker sequence 16S87
ttactagcgattccagcttc

SEQ ID NO: 395 - DNA, 20 bp, Artificial Sequence
  Synthetic blocker sequence 16S88
aaaggataagggttgcgctc

SEQ ID NO: 396 - DNA, 20 bp, Artificial Sequence
  Synthetic blocker sequence 16S89
ttgtagtacgtgtgtagccc

SEQ ID NO: 397 - DNA, 20 bp, Artificial Sequence
  Synthetic blocker sequence 16S90
accggcagtctccttagagt

SEQ ID NO: 398 - DNA, 20 bp, Artificial Sequence
  Synthetic blocker sequence 16S91
ggcagtctcctttgagttcc

SEQ ID NO: 399 - DNA, 20 bp, Artificial Sequence
  Synthetic blocker sequence 16S92
accgtactccccaggcggtc

SEQ ID NO: 400 - DNA, 20 bp, Artificial Sequence
  Synthetic blocker sequence 16S93
gctttcgtgcatgagcgtca

SEQ ID NO: 401 - DNA, 20 bp, Artificial Sequence
  Synthetic blocker sequence 16S94
ctttcgagcctcagcgtcag

SEQ ID NO: 402 - DNA, 20 bp, Artificial Sequence
  Synthetic blocker sequence 16S95
gctttcgcacctgagcgtca

SEQ ID NO: 403 - DNA, 20 bp, Artificial Sequence
  Synthetic blocker sequence 16S96
ctcagtcccagtgtggccga

SEQ ID NO: 404 - DNA, 20 bp, Artificial Sequence
  Synthetic blocker sequence 16S97
ccgtactccccaggcggaat

SEQ ID NO: 405 - DNA, 20 bp, Artificial Sequence
  Synthetic blocker sequence 16S98
tttacaatccgaagaccttc

SEQ ID NO: 406 - DNA, 20 bp, Artificial Sequence
  Synthetic blocker sequence 16S99
gctccccctcgcgggttggc

SEQ ID NO: 407 - DNA, 20 bp, Artificial Sequence
  Synthetic blocker sequence 16S100
gggctttcacatcagactta

SEQ ID NO: 408 - DNA, 20 bp, Artificial Sequence
  Synthetic blocker sequence 23S1
aaggaatttcgctaccttag

SEQ ID NO: 409 - DNA, 20 bp, Artificial Sequence
  Synthetic blocker sequence 23S2
ccgacatcgaggtgccaaac

SEQ ID NO: 410 - DNA, 20 bp, Artificial Sequence
  Synthetic blocker sequence 23S3
ggtcggaacttacccgacaa

SEQ ID NO: 411 - DNA, 20 bp, Artificial Sequence
  Synthetic blocker sequence 23S4
gaactgtctcacgacgttct

SEQ ID NO: 412 - DNA, 20 bp, Artificial Sequence
  Synthetic blocker sequence 23S5
cttttatccgttgagcgatg

SEQ ID NO: 413 - DNA, 20 bp, Artificial Sequence
  Synthetic blocker sequence 23S6
ctttccctcacggtactggt

SEQ ID NO: 414 - DNA, 20 bp, Artificial Sequence
  Synthetic blocker sequence 23S7
accttccagcaccgggcagg

SEQ ID NO: 415 - DNA, 20 bp, Artificial Sequence
  Synthetic blocker sequence 23S8
ggctgcttctaagccaacat

SEQ ID NO: 416 - DNA, 20 bp, Artificial Sequence
  Synthetic blocker sequence 23S9
ggcgaacagcccaacccttg

SEQ ID NO: 417 - DNA, 20 bp, Artificial Sequence
  Synthetic blocker sequence 23S10
gtgagctattacgcactctt

SEQ ID NO: 418 - DNA, 20 bp, Artificial Sequence
  Synthetic blocker sequence 23S11
ttacggccgccgtttactgg

SEQ ID NO: 419 - DNA, 20 bp, Artificial Sequence
  Synthetic blocker sequence 23S12
ggtcctctcgtactaggagc

SEQ ID NO: 420 - DNA, 20 bp, Artificial Sequence
  Synthetic blocker sequence 23S13
ttacgccattcgtgcaggtc

SEQ ID NO: 421 - DNA, 20 bp, Artificial Sequence
  Synthetic blocker sequence 23S14
tttcggggagaaccagctat

SEQ ID NO: 422 - DNA, 20 bp, Artificial Sequence
  Synthetic blocker sequence 23S15
cccttctcccgaagttacgg

SEQ ID NO: 423 - DNA, 20 bp, Artificial Sequence
  Synthetic blocker sequence 23S16
ggcgaccgccccagtcaaac

SEQ ID NO: 424 - DNA, 20 bp, Artificial Sequence
  Synthetic blocker sequence 23S17
tttaaatggcgaacagccat

SEQ ID NO: 425 - DNA, 20 bp, Artificial Sequence
  Synthetic blocker sequence 23S18
gtgagctattacgctttctt

SEQ ID NO: 426 - DNA, 20 bp, Artificial Sequence
  Synthetic blocker sequence 23S19
gacccattatacaaaaggta

SEQ ID NO: 427 - DNA, 20 bp, Artificial Sequence
  Synthetic blocker sequence 23S20
ggtacttagatgtttcagtt

SEQ ID NO: 428 - DNA, 20 bp, Artificial Sequence
  Synthetic blocker sequence 23S21
cctgtgtcggtttgcggtac

SEQ ID NO: 429 - DNA, 20 bp, Artificial Sequence
  Synthetic blocker sequence 23S22
gagaccgccccagtcaaact

SEQ ID NO: 430 - DNA, 20 bp, Artificial Sequence
  Synthetic blocker sequence 23S23
cctcccacctatcctacaca

SEQ ID NO: 431 - DNA, 20 bp, Artificial Sequence
  Synthetic blocker sequence 23S24
agtaaaggttcacggggtct

SEQ ID NO: 432 - DNA, 20 bp, Artificial Sequence
  Synthetic blocker sequence 23S25
gtatttagccttggaggatg

SEQ ID NO: 433 - DNA, 20 bp, Artificial Sequence
  Synthetic blocker sequence 23S26
ccccgttacatcttccgcgc

SEQ ID NO: 434 - DNA, 20 bp, Artificial Sequence
  Synthetic blocker sequence 23S27
ggtatcagcctgttatcccc

SEQ ID NO: 435 - DNA, 20 bp, Artificial Sequence
  Synthetic blocker sequence 23S28
cccaggatgtgatgagccga

SEQ ID NO: 436 - DNA, 20 bp, Artificial Sequence
  Synthetic blocker sequence 23S29
tttcaggttctatttcactc

SEQ ID NO: 437 - DNA, 20 bp, Artificial Sequence
  Synthetic blocker sequence 23S30
gggaccttagctggcggtct

SEQ ID NO: 438 - DNA, 20 bp, Artificial Sequence
  Synthetic blocker sequence 23S31
tagatgctttcagcacttat

SEQ ID NO: 439 - DNA, 20 bp, Artificial Sequence
  Synthetic blocker sequence 23S32
tctcgcagtcaagctccctt

SEQ ID NO: 440 - DNA, 20 bp, Artificial Sequence
  Synthetic blocker sequence 23S33
tttcggagagaaccagctat

SEQ ID NO: 441 - DNA, 20 bp, Artificial Sequence
  Synthetic blocker sequence 23S34
gctagccctaaagctatttc

SEQ ID NO: 442 - DNA, 20 bp, Artificial Sequence
  Synthetic blocker sequence 23S35
cagcattcgcacttctgata

SEQ ID NO: 443 - DNA, 20 bp, Artificial Sequence
  Synthetic blocker sequence 23S36
acggcagatagggaccgaac

SEQ ID NO: 444 - DNA, 20 bp, Artificial Sequence
  Synthetic blocker sequence 23S37
ttacggccgccgtttaccgg

SEQ ID NO: 445 - DNA, 20 bp, Artificial Sequence
  Synthetic blocker sequence 23S38
gcaccgggcaggcgtcacac

SEQ ID NO: 446 - DNA, 20 bp, Artificial Sequence
  Synthetic blocker sequence 23S39
ccgagttctctcaagcgcct

SEQ ID NO: 447 - DNA, 20 bp, Artificial Sequence
  Synthetic blocker sequence 23S40
gcgctacctaaatagctttc

SEQ ID NO: 448 - DNA, 20 bp, Artificial Sequence
  Synthetic blocker sequence 23S41
acctgtgtcggtttggggta

SEQ ID NO: 449 - DNA, 20 bp, Artificial Sequence
  Synthetic blocker sequence 23S42
ctcggttgatttcttttcct

SEQ ID NO: 450 - DNA, 20 bp, Artificial Sequence
  Synthetic blocker sequence 23S43
cattttgcctagttccttca

SEQ ID NO: 451 - DNA, 20 bp, Artificial Sequence
  Synthetic blocker sequence 23S44
ttagcacccgccgtgtgtct

SEQ ID NO: 452 - DNA, 20 bp, Artificial Sequence
  Synthetic blocker sequence 23S45
ggggtctttccgtcctgtcg

SEQ ID NO: 453 - DNA, 20 bp, Artificial Sequence
  Synthetic blocker sequence 23S46
ggagataagcctgttatccc

SEQ ID NO: 454 - DNA, 20 bp, Artificial Sequence
  Synthetic blocker sequence 23S47
ttacgcctttcgtgcgggtc

SEQ ID NO: 455 - DNA, 20 bp, Artificial Sequence
  Synthetic blocker sequence 23S48
ctgtgtttttaataaacagt

SEQ ID NO: 456 - DNA, 20 bp, Artificial Sequence
  Synthetic blocker sequence 23S49
tcgactacgcctttcggcct

SEQ ID NO: 457 - DNA, 20 bp, Artificial Sequence
  Synthetic blocker sequence 23S50
gccctattcagactcgcttt

SEQ ID NO: 458 - DNA, 20 bp, Artificial Sequence
  Synthetic blocker sequence 23S51
ggtttccccattcggaaatc

SEQ ID NO: 459 - DNA, 20 bp, Artificial Sequence
  Synthetic blocker sequence 23S52
tcattctacaaaaggcacgc

SEQ ID NO: 460 - DNA, 20 bp, Artificial Sequence
  Synthetic blocker sequence 23S53
acactgcatcttcacagcga

SEQ ID NO: 461 - DNA, 20 bp, Artificial Sequence
  Synthetic blocker sequence 23S54
tgagtctcgggtggagacag

SEQ ID NO: 462 - DNA, 20 bp, Artificial Sequence
  Synthetic blocker sequence 23S55
ctccgttactctttaggagg

SEQ ID NO: 463 - DNA, 20 bp, Artificial Sequence
  Synthetic blocker sequence 23S56
cagaaccaccggatcactat

SEQ ID NO: 464 - DNA, 20 bp, Artificial Sequence
  Synthetic blocker sequence 23S57
cttcccacatcgtttcccac

SEQ ID NO: 465 - DNA, 20 bp, Artificial Sequence
  Synthetic blocker sequence 23S58
cgaaacagtgctctaccccc

SEQ ID NO: 466 - DNA, 20 bp, Artificial Sequence
  Synthetic blocker sequence 23S59
agccccggtacattttcggc

SEQ ID NO: 467 - DNA, 20 bp, Artificial Sequence
  Synthetic blocker sequence 23S60
ccacatccttttccacttaa

SEQ ID NO: 468 - DNA, 20 bp, Artificial Sequence
  Synthetic blocker sequence 23S61
ctgtgtttttgataaacagt

SEQ ID NO: 469 - DNA, 20 bp, Artificial Sequence
  Synthetic blocker sequence 23S62
cgagttccttaacgagagtt

SEQ ID NO: 470 - DNA, 20 bp, Artificial Sequence
  Synthetic blocker sequence 23S63
ctgggctgtttccctttcga

SEQ ID NO: 471 - DNA, 20 bp, Artificial Sequence
  Synthetic blocker sequence 23S64
catcccggtcctctcgtact

SEQ ID NO: 472 - DNA, 20 bp, Artificial Sequence
  Synthetic blocker sequence 23S65
tgggaaatctcatcttgagg

SEQ ID NO: 473 - DNA, 20 bp, Artificial Sequence
  Synthetic blocker sequence 23S66
gtacaggaatatcaacctgt

SEQ ID NO: 474 - DNA, 20 bp, Artificial Sequence
  Synthetic blocker sequence 23S67
ggaaccaccggatcactaag

SEQ ID NO: 475 - DNA, 20 bp, Artificial Sequence
  Synthetic blocker sequence 23S68
ttacagaacgctcccctacc

SEQ ID NO: 476 - DNA, 20 bp, Artificial Sequence
  Synthetic blocker sequence 23S69
gtctctcgttgagacagtgc

SEQ ID NO: 477 - DNA, 20 bp, Artificial Sequence
  Synthetic blocker sequence 23S70
tgcttctaagccaacctcct

SEQ ID NO: 478 - DNA, 20 bp, Artificial Sequence
  Synthetic blocker sequence 23S71
atcaattaaccttccggcac

SEQ ID NO: 479 - DNA, 20 bp, Artificial Sequence
  Synthetic blocker sequence 23S72
ccattctgagggaacctttg

SEQ ID NO: 480 - DNA, 20 bp, Artificial Sequence
  Synthetic blocker sequence 23S73
aggcatccaccgtgcgccct

SEQ ID NO: 481 - DNA, 20 bp, Artificial Sequence
  Synthetic blocker sequence 23S74
ttggaatttctccgctaccc

SEQ ID NO: 482 - DNA, 20 bp, Artificial Sequence
  Synthetic blocker sequence 23S75
ccgtttcgctcgccgctact

SEQ ID NO: 483 - DNA, 20 bp, Artificial Sequence
  Synthetic blocker sequence 23S76
agatgctttcagcggttatc

SEQ ID NO: 484 - DNA, 20 bp, Artificial Sequence
  Synthetic blocker sequence 23S77
gttacccaaccttcaacctg

SEQ ID NO: 485 - DNA, 20 bp, Artificial Sequence
  Synthetic blocker sequence 23S78
cggtcctccagttagtgtta

SEQ ID NO: 486 - DNA, 20 bp, Artificial Sequence
  Synthetic blocker sequence 23S79
cccgttcgctcgccgctact

SEQ ID NO: 487 - DNA, 20 bp, Artificial Sequence
  Synthetic blocker sequence 23S80
ccggggttcttttcgccttt

SEQ ID NO: 488 - DNA, 20 bp, Artificial Sequence
  Synthetic blocker sequence 23S81
ttcatcgcctctgactgcca

SEQ ID NO: 489 - DNA, 20 bp, Artificial Sequence
  Synthetic blocker sequence 23S82
gaacccttggtcttccggcg

SEQ ID NO: 490 - DNA, 20 bp, Artificial Sequence
  Synthetic blocker sequence 23S83
caaacagtgctctacctcca

SEQ ID NO: 491 - DNA, 20 bp, Artificial Sequence
  Synthetic blocker sequence 23S84
cgattaacgttggacaggaa

SEQ ID NO: 492 - DNA, 20 bp, Artificial Sequence
  Synthetic blocker sequence 23S85
ttttcaacattagtcggttc

SEQ ID NO: 493 - DNA, 20 bp, Artificial Sequence
  Synthetic blocker sequence 23S86
cttagaggcttttcctggaa

SEQ ID NO: 494 - DNA, 20 bp, Artificial Sequence
  Synthetic blocker sequence 23S87
ttggtaagtcgggatgaccc

SEQ ID NO: 495 - DNA, 20 bp, Artificial Sequence
  Synthetic blocker sequence 23S88
ggaccttagctggtggtctg

SEQ ID NO: 496 - DNA, 20 bp, Artificial Sequence
  Synthetic blocker sequence 23S89
gtacaggaatattaacctgt

SEQ ID NO: 497 - DNA, 20 bp, Artificial Sequence
  Synthetic blocker sequence 23S90
cccaggatgcgacgagccga

SEQ ID NO: 498 - DNA, 20 bp, Artificial Sequence
  Synthetic blocker sequence 23S91
ctgcttgtacgtacacggtt

SEQ ID NO: 499 - DNA, 20 bp, Artificial Sequence
  Synthetic blocker sequence 23S92
cccaggatgcgatgagccga

SEQ ID NO: 500 - DNA, 20 bp, Artificial Sequence
  Synthetic blocker sequence 23S93
atcaccgggtttcgggtcta

SEQ ID NO: 501 - DNA, 20 bp, Artificial Sequence
  Synthetic blocker sequence 23S94
gcctttcacccccagccaca

SEQ ID NO: 502 - DNA, 20 bp, Artificial Sequence
  Synthetic blocker sequence 23S95
ttatcgttacttatgtcagc

SEQ ID NO: 503 - DNA, 20 bp, Artificial Sequence
  Synthetic blocker sequence 23S96
tcgactcaccctgccccgat

SEQ ID NO: 504 - DNA, 20 bp, Artificial Sequence
  Synthetic blocker sequence 23S96
gcttatgccattgcactaac

SEQ ID NO: 505 - DNA, 20 bp, Artificial Sequence
  Synthetic blocker sequence 23S97
gctcctacctatcctgtaca

SEQ ID NO: 506 - DNA, 20 bp, Artificial Sequence
  Synthetic blocker sequence 23S98
atcgtaactcgccggttcat

SEQ ID NO: 507 - DNA, 20 bp, Artificial Sequence
  Synthetic blocker sequence 23S99
ttaaagggtggtatttcaag

SEQ ID NO: 508 - DNA, 19 bp, Artificial Sequence
  Sequence of unwanted RNA species
gtcgacacaagggtttgtc

SEQ ID NO: 509 - DNA, 19 bp, Artificial Sequence
  Sequence of unwanted RNA species
ctcgacacaagggtttgtc